(19) United States
(12) Patent Application Publication
(10) Pub. No.: US 2016/0032393 A1
(43) Pub. Date: Feb. 4, 2016

(54) METHODS AND KITS FOR PREDICTING PROGNOSIS OF MULTIPLE SCLEROSIS

(71) Applicant: Tel HaShomer Medical Research Infrastructure and Services Ltd., Ramat-Gan (IL)

(72) Inventors: Anat ACHIRON, Tel-Aviv (IL); Michael GUREVICH, Rechovot (IL)

(73) Assignee: Tel HaShomer Medical Research Infrastructure and Services Ltd., Ramat-Gan (IL)

(21) Appl. No.: 14/882,580

(22) Filed: Oct. 14, 2015

Related U.S. Application Data
(63) Continuation of application No. 12/448,652, filed on Dec. 3, 2009, now abandoned.
(60) Provisional application No. 60/877,680, filed on Dec. 29, 2006.

(51) Int. Cl. C12Q 1/68

(52) U.S. Cl. CPC C12Q 1/6883; C12Q 2600/118; C12Q 2600/158

(57) ABSTRACT

Provided are methods and kits for predicting the prognosis of a subject diagnosed with multiple sclerosis by determining the expression level of polynucleotides which are differentially expressed between subjects diagnosed with multiple sclerosis and having good or poor clinical outcome. Also provided are methods and kits for selecting a treatment regimen of a subject diagnosed with multiple sclerosis.

### RELATED APPLICATIONS

This application is a continuation of U.S. patent application Ser. No. 12/448,652 filed on Dec. 3, 2009, which is a National Phase of PCT Patent Application No. PCT/IL2007/001617 having International Filing Date of Dec. 27, 2007, which claims the benefit of priority of U.S. Provisional Patent Application No. 60/877,680 filed on Dec. 29, 2006. The contents of the above Applications are all incorporated herein by reference.

### SEQUENCE LISTING STATEMENT

The ASCII file, entitled 64258SequenceListing.txt, created on Oct. 14, 2015, comprising 1,548,511 bytes, submitted concurrently with the filing of this application is incorporated herein by reference.

### FIELD AND BACKGROUND OF THE INVENTION

The present invention, in some embodiments thereof, relates to genetic markers which are differentially expressed between multiple sclerosis patients having good or poor clinical outcome, and, more particularly, but not exclusively, to methods and kits using same for predicting the prognosis and selecting treatment regimen for multiple sclerosis.

Multiple sclerosis (MS) is the most common demyelinating disease of the central nervous system (CNS) affecting young adults (disease onset between 20 to 40 years of age) and is the third leading cause for disability after trauma and rheumatic diseases. MS disease prevalence in USA is 120/100,000 (250,000 to 350,000 cases) and in Israel about 30/100,000. The main pathologic finding in MS is the presence of infiltrating mononuclear cells, predominantly T lymphocytes and macrophages, that surpass the blood brain barrier and induce an active inflammation within the brain and spinal cord, attacking the myelin and resulting in gliotic scars and axonal loss. Thus, the multiple inflammatory foci, plaques of demyelination, gliosis and axonal pathology within the brain and spinal cord contribute to the clinical manifestations of neurological disability. The acute and chronic inflammatory processes can be visualized by brain and spinal cord MRI as hyperintense T2 or hypointense T1 lesions.

The etiology of MS is not fully understood. The disease develops in genetically predisposed subjects exposed to yet undefined environmental factors and the pathogenesis involves autoimmune mechanisms associated with autoreactive T cells against myelin antigens. It is well established that not one dominant gene determines genetic susceptibility to develop MS, but rather many genes, each with different influence, are involved. The initial pathogenic process that triggers the disease might be caused by one group of genes, while other groups are probably involved in disease activity and progression (5, 6).

MS is subdivided into several clinical subtypes; when it first presents by new onset of neurological symptoms affecting the CNS and accompanied by demyelinating lesions on brain magnetic resonance imaging (MRI), it is defined as probable MS. A diagnosis of relapsing-remitting (RRMS) definite MS is made when a subject defined as probable MS experiences a second neurological attack. The course of RRMS, which occurs in 85% of patients, is characterized by attacks during which new neurological symptoms and signs appear, or existing neurological symptoms and signs worsen. Usually an attack develops within a period of several days, lasts for 6-8 weeks, and then gradually resolves. During an acute attack, scattered inflammatory and demyelinating CNS lesions produce varying combinations of motor, sensory, coordination, visual, and cognitive impairments, as well as symptoms of fatigue and urinary tract dysfunction. The outcome of an attack is unpredictable in terms of neurological squeal, but it is well established that with each attack, the probability of complete clinical remission decreases, and neurological disability and handicap are liable to develop. In about 15% of patients the disease has a primary progressive course, characterized by gradual onset of neurological symptoms that progress over time, without any attacks. This course appears mostly in patients with disease onset above the age of 40 years and more often in males. The only course of MS in which treatment was effectively established is RRMS. Various immunomodulatory drugs have been shown to reduce the number and severity of acute attacks, and thereby to decrease the accumulation of neurological disability.

Prediction of clinical outcome in MS was reported to relate to different clinical variables such as age at disease onset, gender, and the type of neurological symptomatology presented at onset. Thus, it was suggested that onset age below 35 years, rapid development and regression of initial symptoms, a single symptom at onset, and visual loss as the initial symptom, predicts a good prognosis. On the other hand, the major clinical determinants of more severe disease are male sex, relatively older age at onset, motor or cerebellar symptoms at onset and high annual relapse rate. Brain MRI parameters have also been implicated as important in the evaluation of MS course by measuring disease load over time. Brain atrophy was reported to account for more variance than lesion burden in predicting cognitive impairment. However, all these clinical and radiological variables are limited in the ability to predict disease outcome especially during early stages of the disease. This uncertainty in forecasting disease outcome means that some MS patients who need aggressive treatment do not receive it, while others are unnecessarily treated and as a result are exposed to the risk of side effects without a sound rationale. While peripheral blood genome scale analyses were used to diagnose MS and characterize MS patients in acute relapse or remission (PCT Pub. No. WO03081201A2, EP1532268A2, AU3214604AH, US20060003327A1 to the present inventors; Achiron A, et al., 2004), to date, there are no available genetic markers which can predict the clinical outcome of multiple sclerosis.

### SUMMARY OF THE INVENTION

According to an aspect of some embodiments of the present invention there is provided a method of predicting a prognosis of a subject diagnosed with multiple sclerosis, the method comprising determining in a cell of the subject a level of expression of at least one polynucleotide sequence selected from the group consisting of SEQ ID NOs: 158, 68, 5, 58, 329, 120, 380, 342, 88, 166, 266, 51, 310, 91, 427, 22, 84, 269, 388, 155, 333, 215, 195, 419, 75, 125, 11, 251, 253, 337, 110, 222, 56, 324, 156, 7, 57, 233, 149, 363, 107, 193, 393, 265, 160, 41, 38, 90, 70, 85, 403, 304, 426, 240, 49, 294, 136, 150, 232, 10, 392, 89, 332, 290, 422, 291, 114, 309, 203, 362, 397, 334, 302, 179, 171, 53, 402, 315, 271, 218, 154, 243, 211, 180, 412, 300, 131, 71, 398, 289, 371, 118, 220, 82, 42, 430, 64, 144, 2, 205, 405, 318, 146, 314, 12, 416, 267, 105, 353, 296, 224, 165, 113, 345, 387, 61, 250, 59, 235, 382, 143, 361, 372, 199, 79, 116, 162, 322, 354, 391, 377, 255, 270, 373, 104, 400, 67, 167, 423, 188, 182, 106, 54, 326, 164, 307, 383, 260, 340, 357, 390, 161, 6, 316, 272, 338, 241, 367, 379, 40, 381, 231, 39, 256, 286, 8, 151, 399, 33, 254, 295, 141, 429, 65, 229, 259, 355, 298, 173, 371, 86, 45, 305, 127, 133, 200, 313, 370, 112, 226, 249, 80, 299, 196, 27, 308, 288, 349, 108, 311, 14, 130, 285, 207, 365, 275, 284, 96, 172, 76, 279, 413, 351, 202, 37, 74, 375, 360, 170, 147, 151, 306, 366, 217, 394, 192, 341, 352, 83, 157, 122, 350, 30, 25, 260, 238, 428, 278, 252, 395, 343, 325, 31, 386, 301, 317, 29, 407, 72, 283, 227, 191, 126, 132, 187, 94, 66, 109, 46, 212, 24, 69, 425, 1, 347, 197, 263, 273, 344, 181, 177, 356, 257, 148, 244, 13, 73, 420, 36, 236, 210, 43, 174, 121, 138, 87, 194, 389, 44, 396, 184, 408, 242, 152, 47, 268, 128, 230, 225, 431, 60, 206, 424, 378, 358, 262, 246, 98, 320, 117, 401, 404, 264, 34, 303, 369, 92, 277, 93, 293, 153, 142, 16, 359, 406, 425, 327, 321, 204, 384, 175, 331, 297, 410, 111, 209, 101, 335, 213, 234, 178, 124, 336, 414, 19, 319, 418, 376, 411, 421, 348, 81, 374, 140, 62, 20, 168, 129, 415, 312, 282, 370, 28, 214, 223, 364, 119, 95, 228, 339, 97, 9, 102, 276, 417, 346, 258, 328, 183, 208, 135, 23, 15, 185, 292, 287, 186, 201, 35, 239, 21, 368, 221, 115, 3, 52, 17, 409, 48, 190, 385, 63, 99, 330, 78, 159, 100, 145, 123, 245, 134, 198, 189, 139, 176, 169, 323, 4, 55, 77, 32, 274, 50, 281, 163, 219, 52, 237, 261, 216, 103.

, wherein an alteration above a predetermined threshold in the level of expression of the at least one polynucleotide sequence in the cell of the subject relative to a level of expression of the at least one polynucleotide sequence in a reference cell is indicative of the prognosis of the subject diagnosed with multiple sclerosis.

According to an aspect of some embodiments of the present invention there is provided a method of treating of a subject diagnosed with multiple sclerosis, the method comprising: (a) determining in a cell of the subject a level of expression of at least one polynucleotide sequence selected from the group consisting of SEQ ID NOs:158, 68, 5, 58, 329, 120, 380, 342, 88, 166, 266, 51, 310, 91, 427, 22, 84, 269, 388, 155, 333, 215, 195, 419, 75, 125, 11, 251, 253, 337, 110, 222, 56, 324, 156, 7, 57, 233, 149, 363, 107, 193, 393, 265, 160, 41, 38, 90, 70, 85, 403, 304, 426, 240, 49, 294, 136, 150, 232, 10, 392, 89, 332, 290, 422, 291, 114, 309, 203, 362, 397, 334, 302, 179, 171, 53, 402, 315, 271, 218, 154, 243, 211, 180, 412, 300, 131, 71, 398, 289, 371, 118, 220, 82, 42, 430, 64, 144, 2, 205, 405, 318, 146, 314, 12, 416, 267, 105, 353, 296, 224, 165, 113, 345, 387, 61, 250, 59, 235, 382, 143, 361, 372, 199, 79, 116, 162, 322, 354, 391, 377, 255, 270, 373, 104, 400, 67, 167, 423, 188, 182, 106, 54, 326, 164, 307, 383, 260, 340, 357, 390, 161, 6, 316, 272, 338, 241, 367, 379, 40, 381, 231, 39, 256, 286, 8, 151, 399, 33, 254, 295, 141, 429, 65, 229, 259, 355, 298, 173, 371, 86, 45, 305, 127, 133, 200, 313, 370, 112, 226, 249, 80, 299, 196, 27, 308, 288, 349, 108, 311, 14, 130, 285, 207, 365, 275, 284, 96, 172, 76, 279, 413, 351, 202, 37, 74, 375, 360, 170, 147, 151, 306, 366, 217, 394, 192, 341, 352, 83, 157, 122, 350, 30, 25, 260, 238, 428, 278, 252, 395, 343, 325, 31, 386, 301, 317, 29, 407, 72, 283, 227, 191, 126, 132, 187, 94, 66, 109, 46, 212, 24, 69, 425, 1, 347, 197, 263, 273, 344, 181, 177, 356, 257, 148, 244, 13, 73, 420, 36, 236, 210, 43, 174, 121, 138, 87, 194, 389, 44, 396, 184, 408, 242, 152, 47, 268, 128, 230, 225, 431, 60, 206, 424, 378, 358, 262, 246, 98, 320, 117, 401, 404, 264, 34, 303, 369, 92, 277, 93, 293, 153, 142, 16, 359, 406, 425, 327, 321, 204, 384, 175, 331, 297, 410, 111, 209, 101, 335, 213, 234, 178, 124, 336, 414, 19, 319, 418, 376, 411, 421, 348, 81, 374, 140, 62, 20, 168, 129, 415, 312, 282, 370, 28, 214, 223, 364, 119, 95, 228, 339, 97, 9, 102, 276, 417, 346, 258, 328, 183, 208, 135, 23, 15, 185, 292, 287, 186, 201, 35, 239, 21, 368, 221, 115, 3, 52, 17, 409, 48, 190, 385, 63, 99, 330, 78, 159, 100, 145, 123, 245, 134, 198, 189, 139, 176, 169, 323, 4, 55, 77, 32, 274 50, 281, 163, 219, 52, 237, 261, 216, 103.

wherein an alteration above a predetermined threshold in the level of expression of the at least one polynucleotide sequence in the cell of the subject relative to a level of expression of the at least one polynucleotide sequence in a reference cell is indicative of a prognosis of the subject diagnosed with multiple sclerosis; (b) selecting a treatment regimen based on the prognosis, thereby treating the subject diagnosed with multiple sclerosis.

According to an aspect of some embodiments of the present invention there is provided a kit for predicting a prognosis of a subject diagnosed with multiple sclerosis, comprising no more than 700 isolated nucleic acid sequences, wherein each of the isolated nucleic acid sequences is capable of specifically recognizing at least one polynucleotide sequence selected from the group consisting of SEQ ID NOs:158, 68, 5, 58, 329, 120, 380, 342, 88, 166, 266, 51, 310, 91, 427, 22, 84, 269, 388, 155, 333, 215, 195, 419, 75, 125, 11, 251, 253, 337, 110, 222, 56, 324, 156, 7, 57, 233, 149, 363, 107, 193, 393, 265, 160, 41, 38, 90, 70, 85, 403, 304, 426, 240, 49, 294, 136, 150, 232, 10, 392, 89, 332, 290, 422, 291, 114, 309, 203, 362, 397, 334, 302, 179, 171, 53, 402, 315, 271, 218, 154, 243, 211, 180, 412, 300, 131, 71, 398, 289, 371, 118, 220, 82, 42, 430, 64, 144, 2, 205, 405, 318, 146, 314, 12, 416, 267, 105, 353, 296, 224, 165, 113, 345, 387, 61, 250, 59, 235, 382, 143, 361, 372, 199, 79, 116, 162, 322, 354, 391, 377, 255, 270, 373, 104, 400, 67, 167, 423, 188, 182, 106, 54, 326, 164, 307, 383, 260, 340, 357, 390, 161, 6, 316, 272, 338, 241, 367, 379, 40, 381, 231, 39, 256, 286, 8, 151, 399, 33, 254, 295, 141, 429, 65, 229, 259, 355, 298, 173, 371, 86, 45, 305, 127, 133, 200, 313, 370, 112, 226, 249, 80, 299, 196, 27, 308, 288, 349, 108, 311, 14, 130, 285, 207, 365, 275, 284, 96, 172, 76, 279, 413, 351, 202, 37, 74, 375, 360, 170, 147, 151, 306, 366, 217, 394, 192, 341, 352, 83, 157, 122, 350, 30, 25, 260, 238, 428, 278, 252, 395, 343, 325, 31, 386, 301, 317, 29, 407, 72, 283, 227, 191, 126, 132, 187, 94, 66, 109, 46, 212, 24, 69, 425, 1, 347, 197, 263, 273, 344, 181, 177, 356, 257, 148, 244, 13, 73, 420, 36, 236, 210, 43, 174, 121, 138, 87, 194, 389, 44, 396, 184, 408, 242, 152, 47, 268, 128, 230, 225, 431, 60, 206, 424, 378, 358, 262, 246, 98, 320, 117, 401, 404, 264, 34, 303, 369, 92, 277, 93, 293, 153, 142, 16, 359, 406, 425, 327, 321, 204, 384, 175, 331, 297, 410, 111, 209, 101, 335, 213, 234, 178, 124, 336, 414, 19, 319, 418, 376, 411, 421, 348, 81, 374, 140, 62, 20, 168, 129, 415, 312, 282, 370, 28, 214, 223, 364, 119, 95, 228, 339, 97, 9, 102, 276, 417, 346, 258, 328, 183, 208, 135, 23, 15, 185, 292, 287, 186, 201, 35, 239, 21, 368, 221, 115, 3, 52, 17, 409, 48, 190, 385, 63, 99, 330, 78, 159, 100, 145, 123, 245, 134, 198, 189, 139, 176, 169, 323, 4, 55, 77, 32, 274, 50, 281, 163, 219, 52, 237, 261, 216, 103.

According to an aspect of some embodiments of the present invention there is provided a probeset comprising a plurality of oligonucleotides and no more than 700 oligonucleotides wherein each of the plurality of oligonucleotides is capable of specifically recognizing at least one polynucleotide sequence selected from the group consisting of SEQ ID NOs: 158, 68, 5, 58, 329, 120, 380, 342, 88, 166, 266, 51, 310, 91, 427, 22, 84, 269, 388, 155, 333, 215, 195, 419, 75, 125, 11, 251, 253, 337, 110, 222, 56, 324, 156, 7, 57, 233, 149, 363, 107, 193, 393, 265, 160, 41, 38, 90, 70, 85, 403, 304, 426, 240, 49, 294, 136, 150, 232, 10, 392, 89, 332, 290, 422, 291, 114, 309, 203, 362, 397, 334, 302, 179, 171, 53, 402, 315, 271, 218, 154, 243, 211, 180, 412, 300, 131, 71, 398, 289, 371, 118, 220, 82, 42, 430, 64, 144, 2, 205, 405, 318, 146, 314, 12, 416, 267, 105, 353, 296, 224, 165, 113, 345, 387, 61, 250, 59, 235, 382, 143, 361, 372, 199, 79, 116, 162, 322, 354, 391, 377, 255, 270, 373, 104, 400, 67, 167, 423, 188, 182, 106, 54, 326, 164, 307, 383, 260, 340, 357, 390, 161, 6, 316, 272, 338, 241, 367, 379, 40, 381, 231, 39, 256, 286, 8, 151, 399, 33, 254, 295, 141, 429, 65, 229, 259, 355, 298, 173, 371, 86, 45, 305, 127, 133, 200, 313, 370, 112, 226, 249, 80, 299, 196, 27, 308, 288, 349, 108, 311, 14, 130, 285, 207, 365, 275, 284, 96, 172, 76, 279, 413, 351, 202, 37, 74, 375, 360, 170, 147, 151, 306, 366, 217, 394, 192, 341, 352, 83, 157, 122, 350, 30, 25, 260, 238, 428, 278, 252, 395, 343, 325, 31, 386, 301, 317, 29, 407, 72, 283, 227, 191, 126, 132, 187, 94, 66, 109, 46, 212, 24, 69, 425, 1, 347, 197, 263, 273, 344, 181, 177, 356, 257, 148, 244, 13, 73, 420, 36, 236, 210, 43, 174, 121, 138, 87, 194, 389, 44, 396, 184, 408, 242, 152, 47, 268, 128, 230, 225, 431, 60, 206, 424, 378, 358, 262, 246, 98, 320, 117, 401, 404, 264, 34, 303, 369, 92, 277, 93, 293, 153, 142, 16, 359, 406, 425, 327, 321, 204, 384, 175, 331, 297, 410, 111, 209, 101, 335, 213, 234, 178, 124, 336, 414, 19, 319, 418, 376, 411, 421, 348, 81, 374, 140, 62, 20, 168, 129, 415, 312, 282, 370, 28, 214, 223, 364, 119, 95, 228, 339, 97, 9, 102, 276, 417, 346, 258, 328, 183, 208, 135, 23, 15, 185, 292, 287, 186, 201, 35, 239, 21, 368, 221, 115, 3, 52, 17, 409, 48, 190, 385, 63, 99, 330, 78, 159, 100, 145, 123, 245, 134, 198, 189, 139, 176, 169, 323, 4, 55, 77, 32, 274, 50, 281, 163, 219, 52, 237, 261, 216, 103.

According to some embodiments of the invention, the kit further comprises a reference cell.

According to some embodiments of the invention, each of the isolated nucleic acid sequences or the plurality of oligonucleotides is bound to a solid support.

According to some embodiments of the invention, the plurality of oligonucleotides is bound to the solid support in an addressable location.

According to some embodiments of the invention, the reference cell is of a subject diagnosed with multiple sclerosis which displayed within a period of two years an increase of at least 0.5 point in an Expanded Disability Status Scale (EDSS).

According to some embodiments of the invention, the reference cell is of a subject diagnosed with multiple sclerosis which displayed within a period of two years no change in an Expanded Disability Status Scale (EDSS).

According to some embodiments of the invention, the alteration is upregulation of the expression level of the at least one polynucleotide sequence in the cell of the subject relative to the reference cell, whereas the at least one polynucleotide sequence is selected from the group consisting of SEQ ID NOs:1-193.

According to some embodiments of the invention, the prognosis comprises no change in an Expanded Disability Status Scale (EDSS) of the subject within a period of two years.

According to some embodiments of the invention, the prognosis further comprises no relapses within the period of the two years.

According to some embodiments of the invention, the alteration is upregulation of the expression level of the at least one polynucleotide sequence in the cell of the subject relative to the reference cell, whereas the at least one polynucleotide sequence is selected from the group consisting of SEQ ID NOs:194-431.

According to some embodiments of the invention, the prognosis comprises an increase of at least 0.5 point in an Expanded Disability Status Scale (EDSS) of the subject within a period of at least two years.

According to some embodiments of the invention, detecting the level of expression is effected using an RNA detection method.

According to some embodiments of the invention, the kit further comprising at least one reagent suitable for detecting hybridization of the isolated nucleic acid sequences and at least one RNA transcript corresponding to the at least one polynucleotide sequence selected from the group consisting of SEQ ID NOs: 158, 68, 5, 58, 329, 120, 380, 342, 88, 166, 266, 51, 310, 91, 427, 22, 84, 269, 388, 155, 333, 215, 195, 419, 75, 125, 11, 251, 253, 337, 110, 222, 56, 324, 156, 7, 57, 233, 149, 363, 107, 193, 393, 265, 160, 41, 38, 90, 70, 85, 403, 304, 426, 240, 49, 294, 136, 150, 232, 10, 392, 89, 332, 290, 422, 291, 114, 309, 203, 362, 397, 334, 302, 179, 171, 53, 402, 315, 271, 218, 154, 243, 211, 180, 412, 300, 131, 71, 398, 289, 371, 118, 220, 82, 42, 430, 64, 144, 2, 205, 405, 318, 146, 314, 12, 416, 267, 105, 353, 296, 224, 165, 113, 345, 387, 61, 250, 59, 235, 382, 143, 361, 372, 199, 79, 116, 162, 322, 354, 391, 377, 255, 270, 373, 104, 400, 67, 167, 423, 188, 182, 106, 54, 326, 164, 307, 383, 260, 340, 357, 390, 161, 6, 316, 272, 338, 241, 367, 379, 40, 381, 231, 39, 256, 286, 8, 151, 399, 33, 254, 295, 141, 429, 65, 229, 259, 355, 298, 173, 371, 86, 45, 305, 127, 133, 200, 313, 370, 112, 226, 249, 80, 299, 196, 27, 308, 288, 349, 108, 311, 14, 130, 285, 207, 365, 275, 284, 96, 172, 76, 279, 413, 351, 202, 37, 74, 375, 360, 170, 147, 151, 306, 366, 217, 394, 192, 341, 352, 83, 157, 122, 350, 30, 25, 260, 238, 428, 278, 252, 395, 343, 325, 31, 386, 301, 317, 29, 407, 72, 283, 227, 191, 126, 132, 187, 94, 66, 109, 46, 212, 24, 69, 425, 1, 347, 197, 263, 273, 344, 181, 177, 356, 257, 148, 244, 13, 73, 420, 36, 236, 210, 43, 174, 121, 138, 87, 194, 389, 44, 396, 184, 408, 242, 152, 47, 268, 128, 230, 225, 431, 60, 206, 424, 378, 358, 262, 246, 98, 320, 117, 401, 404, 264, 34, 303, 369, 92, 277, 93, 293, 153, 142, 16, 359, 406, 425, 327, 321, 204, 384, 175, 331, 297, 410, 111, 209, 101, 335, 213, 234, 178, 124, 336, 414, 19, 319, 418, 376, 411, 421, 348, 81, 374, 140, 62, 20, 168, 129, 415, 312, 282, 370, 28, 214, 223, 364, 119, 95, 228, 339, 97, 9, 102, 276, 417, 346, 258, 328, 183, 208, 135, 23, 15, 185, 292, 287, 186, 201, 35, 239, 21, 368, 221, 115, 3, 52, 17, 409, 48, 190, 385, 63, 99, 330, 78, 159, 100, 145, 123, 245, 134, 198, 189, 139, 176, 169, 323, 4, 55, 77, 32, 274, 50, 281, 163, 219, 52, 237, 261, 216, 103.

According to some embodiments of the invention, the kit comprising packaging materials packaging the at least one reagent and instructions for use in determining the prognosis of the subject diagnosed with multiple sclerosis.

According to some embodiments of the invention, the multiple sclerosis is relapsing-remitting multiple sclerosis (RRMS).

According to some embodiments of the invention, the at least one polynucleotide sequence is selected from the group consisting of SEQ ID NOs: 156, 143, 127, 46, 311, 140, 74, 276, 180, 182, 191, 61, 306, 115, 97, 303, 272, 50, 16, 63, 117, 406, 423, 128, 277, 47, 17, 424, 418, 190, 139, 102, 103 and 325.

According to some embodiments of the invention, the at least one polynucleotide sequence is selected from the group consisting of SEQ ID NOs:127, 423, 16, 17, 424, 190 and 325.

According to some embodiments of the invention, the at least one polynucleotide comprises the 7 polynucleotides set forth by SEQ ID NOs:127, 423, 16, 17, 424, 190 and 325.

According to some embodiments of the invention, the cell of the subject is a blood cell.

According to some embodiments of the invention, the at least one polynucleotide sequence is set forth by SEQ ID NO:158.

According to some embodiments of the invention, the at least one polynucleotide comprises the polynucleotide sequences set forth by SEQ ID NOs:158, 68, 5, 58, 329 and 120.

According to some embodiments of the invention, detecting the level of expression is effected using a protein detection method.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of the study design. Overview of the strategy used for the identification and validation of predictive clinical outcome gene-expression signature in RRMS using the signature support vector machine (SVM) in combination with Forward feature selection algorithm were applied (http://ro(dot)utia(dot)cz/fs/fs_algorithms (dot)html), (12, 13).

FIG. 2 depicts a heatmap of 431 differentiating genes between poor and good clinical outcome of RRMS patients. Each row of the heatmap represents a gene and each column represents a patient's sample. Genes with increased expression (upregulation) are shown in progressively brighter shades of red, and genes with decreased expression (downregulation) are shown in progressively darker shades of green. The bottom matrix shows corresponding clinical outcome attributes marked in black when applicable. EDSS (Expanded Disability Status Scale) scores were determined in RRMS patients at the recruitment to study and during a two-years follow-up; EDSS 0—no change in EDSS score; Delta EDSS neg (negative)—improvement; Delta EDSS pos (positive)—deterioration; Relapse—attack;

FIG. 3 depicts a functional annotation histogram of some of the differentiating genes between poor and good clinical outcome of RRMS. Distribution of differentiating gene expression signature according to biologically relevant functional groups. Numbers represent the number of genes from the differentiating signature which belong to each functional annotation;

FIG. 4 is a graph depicting an overabundance analysis of the differentiating genes between poor and good clinical outcome of RRMS. Actual number of genes (blue line) is significantly more abundant than expected (red line) for TNoM statistical test. X-axis denotes p-value; y-axis denotes number of genes;

FIG. 5 is a graph depicting the Leave-One-Out-Cross-Validation (LOOCV) classification. Division of errors between patients with good and poor clinical outcome of RRMS using TNoM, Info and t-test demonstrated high classification rate of 90% at p<0.0001. X-axis denotes p value; y-axis denotes error rate in %.

FIG. 6 is a graph depicting the predictive classification chart of the differentiating genes between poor and good clinical outcome of RRMS. The classification rate of 29 predictive genes is demonstrated. Highest classification rate is achieved using only 7 genes, yet according to the feature selection algorithm, genes are added to the subset as long as the classification rate is not decreased. Y axis denotes classification rate; x axis denotes the number of genes;

FIG. 7 depicts gene enrichment of the differentiating genes between poor and good clinical outcome of RRMS. Direction of an over-expressed (1) or down-expressed (−1) gene is demonstrated in the enriched groups within the poor vs. good outcome signature;

FIGS. 8a-8c are infograms depicting the representation of genes related to specific biological processes in the 431 probesets of the present invention (shown in FIG. 2; SEQ ID NOs:1-431) which are differentially expressed between MS subjects with good or poor clinical outcome. FIG. 8a—A matrix of gene sets vs. arrays (each array represents an MS subject), where a colored entry indicates that the genes in the gene set had significantly changed in a coordinated fashion in the respective array (red—increased, green—decreased, black—not changed) as compared to the expected number of genes in each biological process as calculated using the Genomica software (http://genomica(dot)weizmann(dot)ac(dot)il). The names of the biological processes are shown on the top index and the MS subject reference numbers are shown on the right index of FIG. 8b. FIG. 8b shows individual clinical outcome attributes that each array belongs to. The clinical outcome attributes include: EDSS 0 (no change in EDSS score), delta EDSS neg (negative; improvement), delta EDSS pos (positive; deterioration), poor outcome (poor clinical outcome as determined during two years), and relapse (attack). The color index is a follows: pink=presence of parameter; white—absence of parameter. FIG. 8c—a Module map demonstrating overall clinical outcome attributes in which gene sets were significantly enriched. Red—the number of genes in the specific biological process is higher than expected; green—the number of genes in the specific biological process is lower than expected; and black—the number of genes in the specific biological process is as expected. Note the enrichment of zinc-ion binding gene set for patients with relapses (MS subjects Nos. 88, 93, 99, 109, 110, 173, 210, 213, 215) and cytokine activity gene set for patients with stable disease (no change in neurological disability, EDSS=0; MS subjects Nos. 23, 25, 31, 34, 89, 119, 158).

FIG. 9 is a schematic model depicting the reconstructed zinc-ion binding pathway. Pathway analysis performed using genes from the predictive signature (yellow circles) and genes brought into the pathway based on literature known relationships according to PathwayArchitect software (green circles). Arrows indicate regulatory interactions confirmed by literature database, dashed arrows indicate suggested gene interactions;

FIG. 10 is a schematic model depicting the reconstructed cytokine activity pathway. Pathway analysis performed using genes from the predictive signature (gray circles) and genes brought into the pathway based on literature known relationships according to PathwayArchitect software (blue circles). Arrows indicate regulatory interactions confirmed by literature database, dashed arrows indicate suggested gene interactions;

FIG. 11 depicts the gene expression regulatory network module. The single gene expression module from the gene expression regulatory network of 431 differentiating genes is demonstrated. Each node in the regulation tree represents a regulating gene. The expression of the regulating genes themselves is shown below their node. Cluster of gene expression profiles (rows represent genes, columns—patients arrays) arranged according to the regulation tree. Note that zinc-ion binding related genes KLF4 (regulating gene, arrow on the left) and S100B (regulated gene, arrow on the right) belong to same regulatory module.

FIG. 12 is a graph depicting the average error of the predictive ability of combination of 431 differentiating genes.

### DESCRIPTION OF SPECIFIC EMBODIMENTS OF THE INVENTION

The present invention, in some embodiments thereof, relates to genetic markers which are differentially expressed between subjects diagnosed with multiple sclerosis and having good or poor clinical outcome which can be use to predict the prognosis of a subject diagnosed with multiple sclerosis. Specifically, but not exclusively, the present invention can be used to treat multiple sclerosis by selecting a suitable treatment regimen based on the predicted clinical outcome of the subject.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the invention to practice, the present inventors have uncovered differentially expressed genes which are associated with poor or good clinical outcome of multiple sclerosis and which can be used to predict the prognosis of a subject diagnosed with multiple sclerosis.

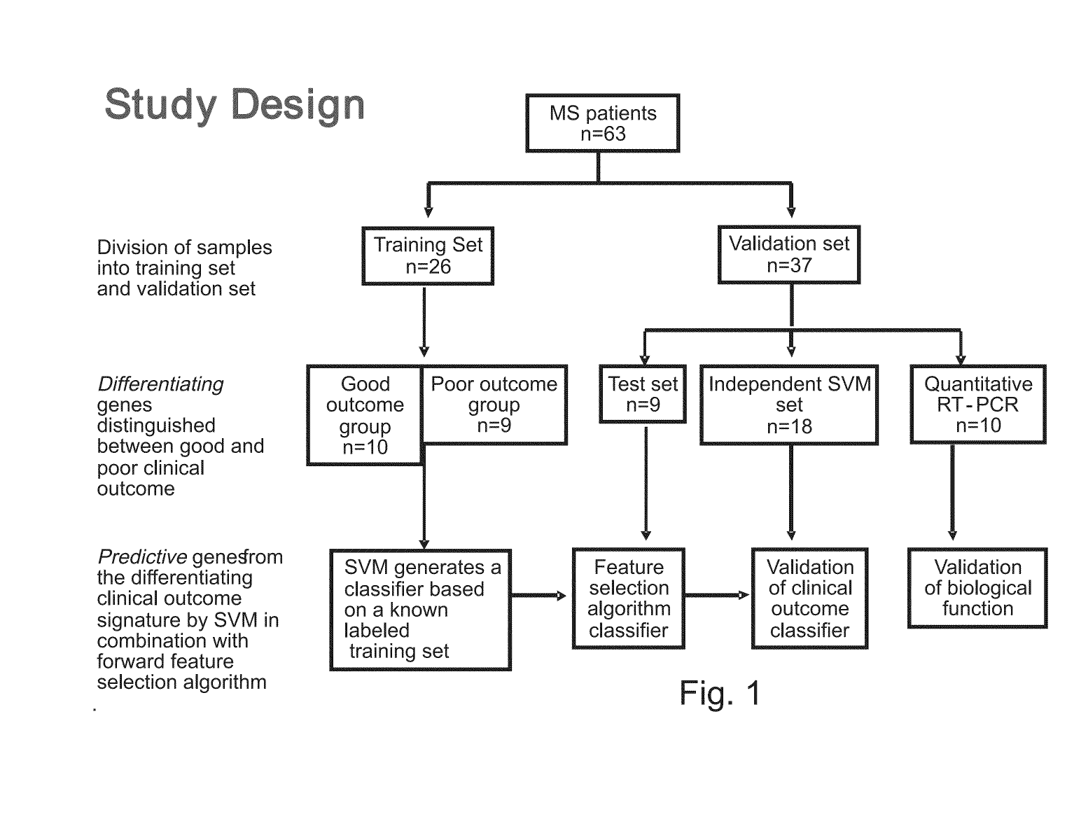

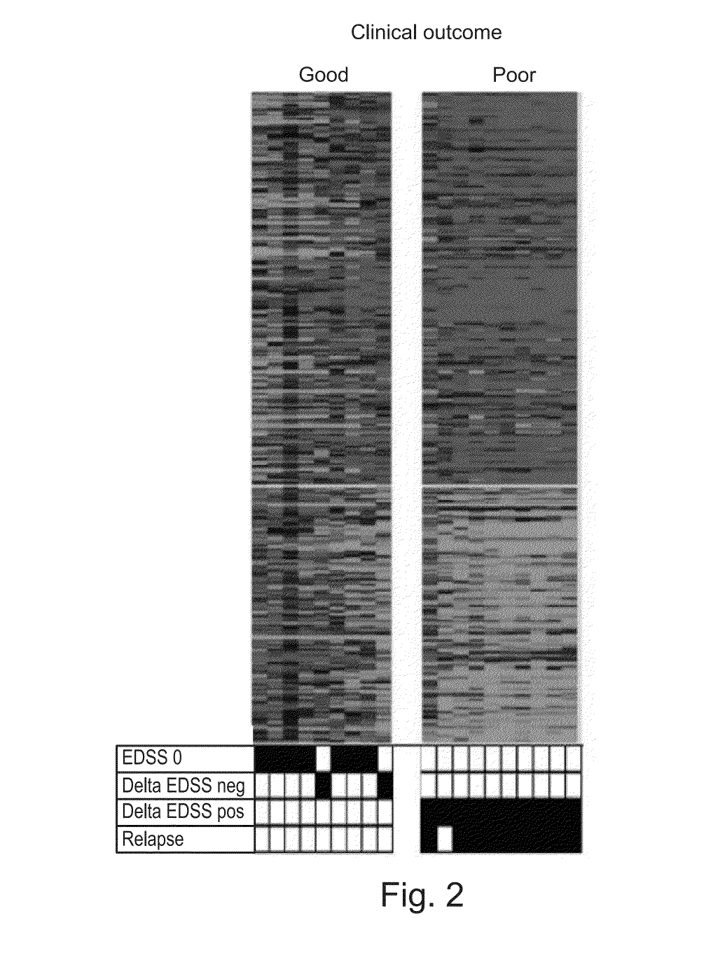

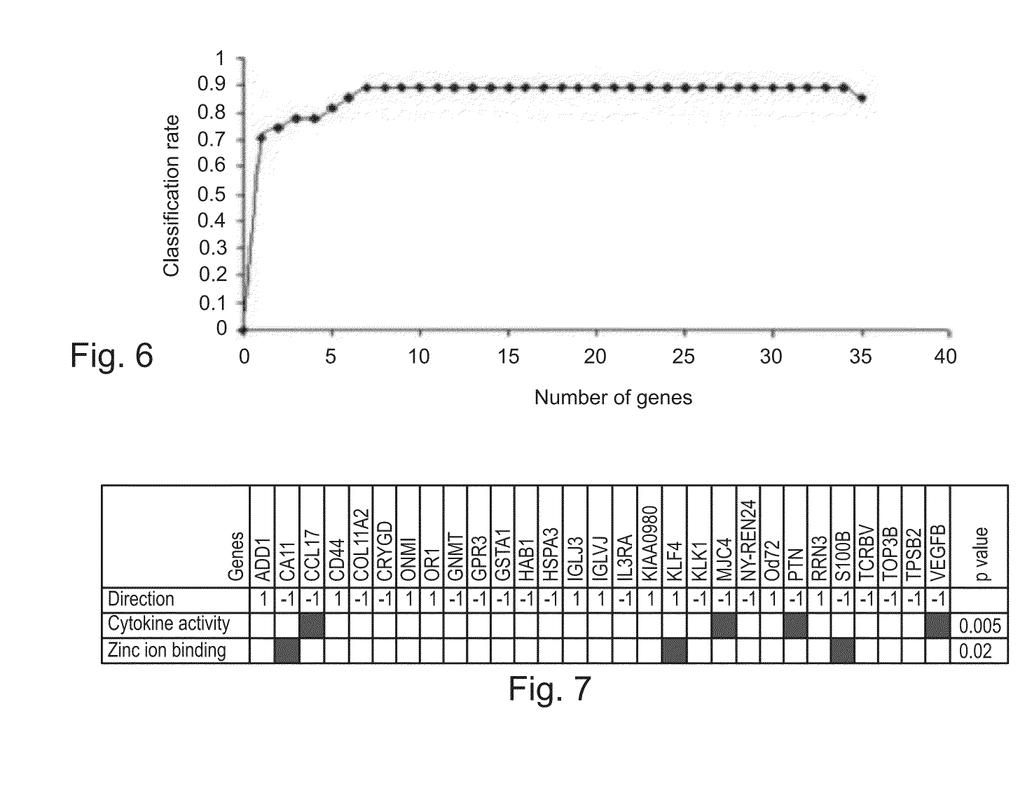

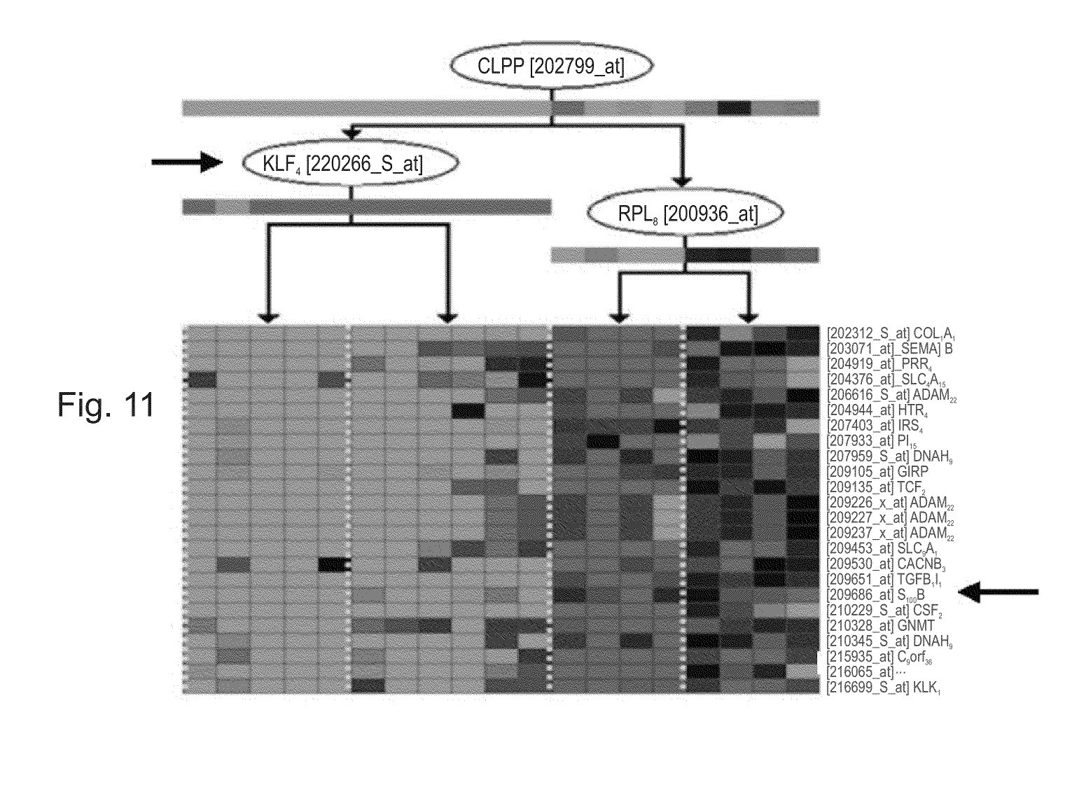

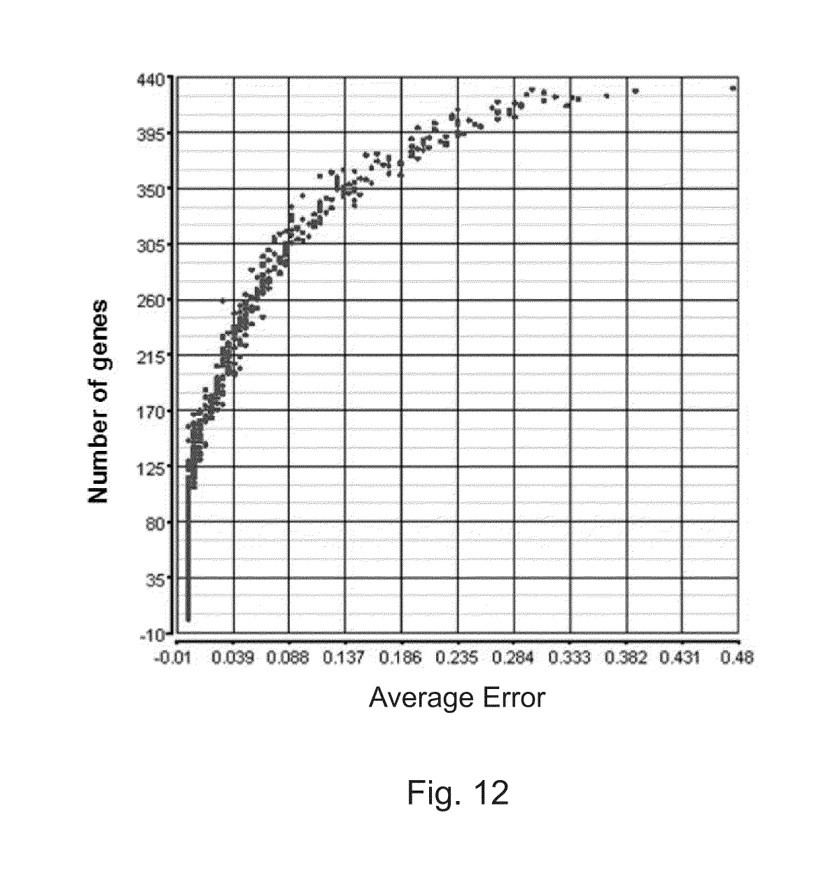

As is shown in the Examples section which follows, the present inventors have identified 431 genetic markers which are differentially expressed between relapsing-remitting MS (RRMS) patients with good or poor clinical outcome as established after a 2-year follow-up (FIGS. 2, 3, 4, 5 and Table 2 and Example 1 of the Examples section which follows). Moreover, when supervised learning and feature selection algorithms were applied and validated in an independent set of 27 samples from a prospective cohort of RRMS patients, an optimal set of 34 gene transcripts was depicted as a clinical outcome predictive gene expression signature with a classification accuracy of 88.9% (FIGS. 1, 6, Table 3, Example 2 of the Examples section which follows). This predictive signature was enriched in genes biologically related to zinc-ion binding and cytokine activity regulation pathways (FIGS. 7, 8a-8c, 9, 10, 11, Example 3 of the Examples section which follows). In addition, when the SVM software based on RBF kernel were applied on a training set of 26 subjects optimal sets of genes which can predict the prognosis of RRMS patients with 100% accuracy (average error of “0”) were depicted (FIG. 12, Table 4, Example 4 of the Examples section which follows). Altogether, these results demonstrate for the first time that genetic markers can discriminate between MS patients with good and poor clinical outcome, and suggest the use of such differentially expressed genes in predicting the prognosis of multiple sclerosis.

Thus, according to one aspect of the invention there is provided a method of predicting a prognosis of a subject diagnosed with multiple sclerosis. The method is effected by determining in a cell of the subject a level of expression of at least one polynucleotide sequence selected from the group consisting of SEQ ID NOs:1-431, wherein an alteration above a predetermined threshold in the level of expression of the at least one polynucleotide sequence in the cell of the subject relative to a level of expression of the at least one polynucleotide sequence in a reference cell is indicative of the prognosis of the subject diagnosed with multiple sclerosis.

As used herein, the phrase “a subject diagnosed with multiple sclerosis” refers to a mammal, preferably a human being, who is diagnosed with definite multiple sclerosis, e.g., a subject who experienced at least two neurological attacks affecting the CNS and accompanied by demyelinating lesions on brain magnetic resonance imaging (MRI). It will be appreciated that the disease course of patients diagnosed with multiple sclerosis can be a relapsing-remitting multiple sclerosis (RRMS) (occurring in 85% of the patients) or a progressive multiple sclerosis (occurring in 15% of the patients). According to an embodiment of the invention, the subject is diagnosed with RRMS.

As used herein, the phrase “predicting a prognosis” refers to determining the clinical outcome of the subject diagnosed with multiple sclerosis, e.g., determining the risk of deterioration in terms of neurological disability and/or the total number of relapses. For example, a good clinical outcome (good prognosis) of a subject diagnosed with multiple sclerosis is no deterioration in the neurological disability [no change in the Expanded Disability Status Scale (EDSS) score] and no relapses for a period of at least 24 months; a poor clinical outcome (poor prognosis) is a deterioration in the neurological disability (the EDSS score is increased by at least 0.5 point) within a period of at least 24 months, either with or without relapses; an intermediate clinical outcome (intermediate prognosis) is no deterioration in the neurological disability (no change in the EDSS score) and yet at least one relapse during a period of at least 24 months.

As mentioned, the method according to this aspect of the invention is effected by determining in a cell of the subject a level of expression of at least one polynucleotide sequence selected from the group consisting of SEQ ID NOs:1-431.

According to an embodiment of the invention, the method is effected by determining in a cell of the subject a level of expression of at least two, at least three, at least four, at least five, at least six (e.g., six), at least seven (e.g., seven), at least eight, at least nine, at least 10 polynucleotide sequences, at least 20, at least 30, at least 40, at least 50 polynucleotide sequences selected from the group consisting of SEQ ID NOs:1-431, wherein an alteration above a predetermined threshold in the level of expression of each of the polynucleotide sequences in the cell of the subject relative to a level of expression of the same polynucleotide sequences in a reference cell is indicative of the prognosis of the subject diagnosed with multiple sclerosis.

As used herein, the phrase “level of expression” refers to the degree of gene expression and/or gene product activity in a specific cell. For example, up-regulation or down-regulation of various genes can affect the level of the gene product (i.e., RNA and/or protein) in a specific cell.

As used herein the phrase “a cell of the subject” refers to any cell, cell content and/or cell secreted content which contains RNA and/or proteins of the subject. Examples include a blood cell, a bone marrow cell, a cell obtained from any tissue biopsy [e.g., cerebrospinal fluid, (CSF), brain biopsy], body fluids such as plasma, serum, saliva, spinal fluid, lymph fluid, the external sections of the skin, respiratory, intestinal, and genitourinary tracts, tears, saliva, sputum and milk. According to an embodiment of the invention, the cell is a blood cell (e.g., white blood cells, macrophages, B- and T-lymphocytes, monocytes, neutrophiles, eosinophiles, and basophiles) which can be obtained using a syringe needle from a vein of the subject. It should be noted that a “cell of the subject” may also optionally comprise a cell that has not been physically removed from the subject (e.g., in vivo detection).

According to an embodiment of the invention, the white blood cell comprises peripheral blood mononuclear cells (PBMC). The phrase, “peripheral blood mononuclear cells (PBMCs)” as used herein, refers to a mixture of monocytes and lymphocytes. Several methods for isolating white blood cells are known in the art. For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants (e.g., red blood cells) prior to determining the expression level of the polynucleotide(s) therein.

It will be appreciated that the cell of the subject can be obtained at any time, e.g., immediately after an attack or during remission.

According to an embodiment of the invention, detecting the level of expression of the polynucleotide sequences of the invention is effected using RNA or protein molecules which are extracted from the cell of the subject.

Methods of extracting RNA or protein molecules from cells of a subject are well known in the art.

Once obtained, the RNA or protein molecules can be characterized for the expression and/or activity level of various RNA and/or protein molecules using methods known in the arts.

Non-limiting examples of methods of detecting RNA molecules in a cell sample include Northern blot analysis, RT-PCR, RNA in situ hybridization (using e.g., DNA or RNA probes to hybridize RNA molecules present in the cells or tissue sections), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface [e.g., a glass wafer) with addressable location, such as Affymetrix microarray (Affymetrix®, Santa Clara, Calif.)].

Non-limiting examples of methods of detecting the level and/or activity of specific protein molecules in a cell sample include Enzyme linked immunosorbent assay (ELISA), Western blot analysis, radio-immunoassay (RIA), Fluorescence activated cell sorting (FACS), immunohistochemical analysis, in situ activity assay (using e.g., a chromogenic substrate applied on the cells containing an active enzyme), in vitro activity assays (in which the activity of a particular enzyme is measured in a protein mixture extracted from the cells).

For example, in case the detection of the expression level of a secreted protein is desired, ELISA assay may be performed on a sample of fluid obtained from the subject (e.g., serum), which contains cell-secreted content.

As used herein the phrase “reference cell” refers to any cell as described hereinabove of a subject diagnosed with multiple sclerosis and having a known clinical outcome (e.g., poor, good or intermediate clinical outcome) as determined during a predetermined period of time, such as 2 years. Such a reference cell can be a blood cell, a bone marrow cell, a cell obtained from any tissue biopsy (e.g., CSF, brain biopsy), body fluids such as plasma, serum, saliva, spinal fluid, lymph fluid, the external sections of the skin, respiratory, intestinal, and genitourinary tracts, tears, saliva, sputum and milk. It will be appreciated that the level of expression of the above referenced polynucleotides/polypeptides may be obtained from scientific literature.

According to an embodiment of the invention, the reference cell comprises a cell of a subject diagnosed with multiple sclerosis and having a good clinical outcome. For example, such a reference cell can be a blood cell of a subject which exhibited no deterioration in the neurological disability (no change in the EDSS score) and no relapses during a period of at least 24 months.

Since as is shown in Table 2 and is described in Example 1 of the Examples section which follows, 238 polynucleotide sequences displayed elevated expression in the MS patients having poor clinical outcome relative to the MS patients having good clinical outcome, in order to predict the prognosis of a subject diagnosed with multiple sclerosis, the level of expression of at least one polynucleotide sequence selected from the group consisting of SEQ ID NOs:194-431 is determined and compared to the level of expression of the same polynucleotide sequences in a reference cell derived from a subject diagnosed with MS and having good clinical outcome, wherein an upregulation (increase) in the expression level of the at least one polynucleotide sequence above a predetermined threshold relative to the reference cell is indicative of a poor prognosis (poor clinical outcome).

Additionally or alternatively, since as is further shown in Table 2 and is described in Example 1 of the Examples section which follows, the level of expression of 193 polynucleotide sequences was downregulated in the MS patients having poor clinical outcome relative to the MS patients having good clinical outcome, in order to predict the prognosis of a subject diagnosed with multiple sclerosis, the level of expression of at least one polynucleotide sequence selected from the group consisting of SEQ ID NOs:1-193 is determined and compared to the level of expression of the same polynucleotide sequences in a reference cell derived from a subject diagnosed with MS and having good clinical outcome, wherein downregulation (decrease) in the expression level of the at least one polynucleotide sequence above a predetermined threshold relative to the reference cell is indicative of a poor prognosis (poor clinical outcome).

According to an embodiment of the invention, the reference cell comprises a cell of a subject diagnosed with multiple sclerosis and having a poor clinical outcome. For example, such a reference cell can be a blood cell of a subject which exhibited deterioration in the neurological disability (at least 0.5 point in the EDSS score) during a period of at least 24 months, either with or without relapses.

Since as is shown in Table 2 and is described in Example 1 of the Examples section which follows, the expression level of 238 polynucleotide sequences was downregulated in MS patients having good clinical outcome relative to the level of expression in MS patients having poor clinical outcome, in order to predict the prognosis of a subject diagnosed with MS, the level of expression of at least one polynucleotide sequence selected from the group consisting of SEQ ID NOs:194-431 is determined and compared to the level of expression of the same polynucleotide sequences in a reference cell derived from an MS patient with poor clinical outcome, wherein downregulation (decrease) in the expression level of the at least one polynucleotide sequence above a predetermined threshold relative to the reference cell is indicative of a good prognosis (good clinical outcome).

Additionally or alternatively, since as is shown in Table 2 and is described in Example 1 of the Examples section which follows, the level of expression of 193 polynucleotide sequences was upregulated in the MS patients having good clinical outcome relative to the level of expression in MS patients having poor clinical outcome, in order to predict the prognosis of a subject diagnosed with MS, the level of expression of at least one polynucleotide sequence selected from the group consisting of SEQ ID NOs:1-193 is determined and compared to the level of expression of the same polynucleotide sequences in a reference cell derived from an MS patient with poor clinical outcome, wherein upregulation (increase) in the expression level of the at least one polynucleotide sequence above a predetermined threshold relative to the reference cell is indicative of a good prognosis (good clinical outcome).

It will be appreciated that the reference cell can be also a cell of a subject diagnosed with multiple sclerosis and having an intermediate clinical outcome. For example, such a reference cell can be a blood cell of a subject which exhibited no deterioration in the neurological disability (no change in the EDSS score), yet experienced at least one relapse during a period of at least 24 months.

As is further shown in FIG. 6 and Table 3 and is described in Example 2 of the Examples section which follows the present inventors have uncovered that 34 out of the 431 differentiating genetic markers are capable of classifying MS patients to those having good or poor clinical outcome with a classification accuracy of at least 89%.

Thus, according to an embodiment of the invention, the at least one polynucleotide which expression level is determined in the cell of the subject diagnosed with MS is selected from the polynucleotides set forth in SEQ ID NOs:156, 143, 127, 46, 311, 140, 74, 276, 180, 182, 191, 61, 306, 115, 97, 303, 272, 50, 16, 63, 117, 406, 423, 128, 277, 47, 17, 424, 418, 190, 139, 102, 103 and 325.

According to an embodiment of the invention, downregulation of the expression level of at least one polynucleotide sequence of the polynucleotides set forth by SEQ ID NOs:156, 143, 127, 46, 140, 74, 180, 182, 191, 61, 115, 97, 50, 16, 63, 117, 128, 47, 17, 190, 139, 102 and 103, and/or upregulation of at least one polynucleotide sequence of the polynucleotides set forth by SEQ ID NOs:311, 276, 306, 303, 272, 406, 423, 277, 424, 418, and 325, relative to a reference cell of a subject diagnosed with MS and having good clinical outcome is indicative of poor prognosis of the subject diagnosed with MS.

On the other hand, upregulation of the expression level of at least one polynucleotide sequence of the polynucleotides set forth by SEQ ID NOs:156, 143, 127, 46, 140, 74, 180, 182, 191, 61, 115, 97, 50, 16, 63, 117, 128, 47, 17, 190, 139, 102 and 103, and/or downregulation of at least one polynucleotide sequence of the polynucleotides set forth by SEQ ID NOs:311, 276, 306, 303, 272, 406, 423, 277, 424, 418, and 325 relative to a reference cell of a subject diagnosed with MS and having poor clinical outcome is indicative of good prognosis of the subject diagnosed with MS.

As is further shown in FIG. 6 and Table 3 and is described in Example 2 of the Examples section which follows, classification rate of 85.2% was achieved using markers of the following 6 genes: TPSB2 (SEQ ID NO:127), IGLJ3 (SEQ ID NO:423), HAB1 (SEQ ID NOs:16 and/or 17), RRN3 (SEQ ID NO:424), COL11A2 (SEQ ID NO:190) and KLF4 (SEQ ID NO:325).

According to an embodiment of the invention, upregulation of the expression level of IGLJ3 (SEQ ID NO:423), RRN3 (SEQ ID NO:424) and KLF4 (SEQ ID NO:325) and downregulation of TPSB2 (SEQ ID NO:127), HAB1 (SEQ ID NOs:16 and/or 17) and COL11A2 (SEQ ID NO:190) relative to a reference cell of a subject diagnosed with MS and having good clinical outcome is indicative of poor prognosis of the subject diagnosed with MS.

On the other hand, downregulation of the expression level of IGLJ3 (SEQ ID NO:423), RRN3 (SEQ ID NO:424) and KLF4 (SEQ ID NO:325) and upregulation of TPSB2 (SEQ ID NO:127), HAB1 (SEQ ID NOs:16 and/or 17) and COL11A2 (SEQ ID NO:190) relative to a reference cell of a subject diagnosed with MS and having poor clinical outcome is indicative of good prognosis of the subject diagnosed with MS.

As is further shown in FIG. 6 and Table 3 and is described in Example 2 of the Examples section which follows, classification rate of 70.4% was achieved using only one gene (RRN3; SEQ ID NO:424). Thus, according to an embodiment of the invention upregulation of the expression level of RRN3 (SEQ ID NO:424) relative to a reference cell of a subject diagnosed with MS and having good clinical outcome is indicative of a poor prognosis of a subject diagnosed with MS. On the other hand, downregulation of the expression level of RRN3 (SEQ ID NO:424) relative to a reference cell of a subject diagnosed with MS and having poor clinical outcome is indicative of a good prognosis of a subject diagnosed with MS.

As is further shown in FIG. 12 and Table 4 (Example 4) and mentioned hereinabove, when the SVM based on RBF kernel were applied on 26 subjects optimal sets of genes which can predict the prognosis of RRMS patients with 100% accuracy (average error of “0”) were depicted.

Thus, according to an embodiment of the invention the at least one polynucleotide which expression level is determined in the cell of the subject diagnosed with MS is set forth by SEQ ID NO:158.

According to an embodiment of the invention, the polynucleotide sequences which expression level are determined in the cell of the subject diagnosed with MS are those depicted in any of the following groups of row numbers of Table 4 in Example 4 of the Examples section which follows: rows 1-2; rows 1-3; rows 1-4; rows 1-5; rows 1-6; rows 1-7; rows 1-8; rows 1-9; rows 1-10; rows 1-11; rows 1-12; rows 1-13; rows 1-14; rows 1-15; rows 1-16; rows 1-17; rows 1-18; rows 1-19; rows 1-20; rows 1-21; rows 1-22; rows 1-23; rows 1-24; rows 1-25; rows 1-26; rows 1-27; rows 1-28; rows 1-29; rows 1-30; rows 1-31; rows 1-32; rows 1-33; rows 1-34; 1-35; rows 1-36; rows 1-37; rows 1-38; rows 1-39; rows 1-40; rows 1-41; rows 1-42; rows 1-43; rows 1-44; rows 1-45; rows 1-46; rows 1-47; rows 1-48; rows 1-49; rows 1-50; rows 1-51; rows 1-52; rows 1-53; rows 1-54; 1-55; rows 1-56; rows 1-57; rows 1-58; rows 1-59; rows 1-60; rows 1-61; rows 1-62; rows 1-63; rows 1-64; rows 1-65; rows 1-66; rows 1-67; rows 1-68; rows 1-69; rows 1-70; rows 1-71; rows 1-72; rows 1-73; rows 1-74; 1-75; rows 1-76; rows 1-77; rows 1-78; rows 1-79; rows 1-80; rows 1-81; rows 1-82; rows 1-83; rows 1-84; rows 1-85; rows 1-86; rows 1-87; rows 1-88; rows 1-89; rows 1-90; rows 1-91; rows 1-92; rows 1-93; rows 1-94; 1-95; rows 1-96; rows 1-97; rows 1-98; rows 1-99; rows 1-100; rows 1-101; rows 1-102; rows 1-103; rows 1-104; rows 1-105; rows 1-106; rows 1-107; rows 1-109; rows 1-110; rows 1-112; rows 1-113; rows 1-114; rows 1-116; rows 1-122; 1-124; rows 1-125; rows 1-126; rows 1-129; rows 1-146; rows 1-157.

As is further shown in Table 4 (Example 4) other groups of genes can predict the prognosis of RRMS patients with 99.5% accuracy (average error of “0.005”).

According to an embodiment of the invention, the polynucleotide sequences which expression level are determined in the cell of the subject diagnosed with MS are those depicted in any of the following groups of row numbers of Table 4 in Example 4 of the Examples section which follows: rows 1-108; rows 1-111; rows 1-115; rows 1-117; rows 1-118; rows 1-119; rows 1-120; rows 1-121; rows 1-123; rows 1-127; rows 1-128; rows 1-131; rows 1-132; rows 1-133; 1-135; rows 1-137; rows 1-138; rows 1-139; rows 1-141; rows 1-144; rows 1-148; rows 1-150; rows 1-152; rows 1-153; rows 1-154; rows 1-158; rows 1-160; rows 1-167.

As is further shown in Table 4 (Example 4) other groups of genes can predict the prognosis of RRMS patients with 99% accuracy (average error of “0.01”).

According to an embodiment of the invention, the polynucleotide sequences which expression level are determined in the cell of the subject diagnosed with MS are those depicted in any of the following groups of row numbers of Table 4 in Example 4 of the Examples section which follows: rows 1-130; rows 1-134; rows 1-136; rows 1-140; rows 1-145; rows 1-147; 1-149; rows 1-151; rows 1-155; rows 1-156; rows 1-159; rows 1-162; rows 1-168; rows 1-170.

As is further shown in Table 4 (Example 4) other groups of genes can predict the prognosis of RRMS patients with 98-98.5% accuracy (average error of “0.015-0.02”).

According to an embodiment of the invention, the polynucleotide sequences which expression level are determined in the cell of the subject diagnosed with MS are those depicted in any of the following groups of row numbers of Table 4 in Example 4 of the Examples section which follows: rows 1-142; rows 1-143; rows 1-161; rows 1-163; rows 1-164; rows 1-165; rows 1-166; rows 1-169; rows 1-172; rows 1-173; rows 1-174; rows 1-177; rows 1-178; rows 1-179; rows 1-181; rows 1-187.

As is further shown in Table 4 (Example 4) other groups of genes can predict the prognosis of RRMS patients with 95-97.5% accuracy (average error of “0.025-0.05”).

According to an embodiment of the invention, the polynucleotide sequences which expression level are determined in the cell of the subject diagnosed with MS are those depicted in any of the following groups of row numbers of Table 4 in Example 4 of the Examples section which follows: rows 1-171; rows 1-176; rows 1-180; rows 1-183; rows 1-184; rows 1-185; rows 1-186; rows 1-188; rows 1-189; rows 1-190; rows 1-191; rows 1-192; rows 1-193; rows 1-194; rows 1-195; rows 1-196; rows 1-197; rows 1-198; rows 1-199; 1-200; rows 1-201; rows 1-202; rows 1-203; rows 1-204; rows 1-205; rows 1-206; rows 1-207; rows 1-208; rows 1-209; rows 1-210; rows 1-211; rows 1-212; rows 1-213; rows 1-214; rows 1-215; rows 1-216; rows 1-217; rows 1-218; rows 1-219; rows 1-220; rows 1-221; rows 1-222; rows 1-223; rows 1-224; rows 1-225; rows 1-226; rows 1-227; rows 1-228; rows 1-229; rows 1-230; rows 1-231; rows 1-232; rows 1-233; rows 1-234; rows 1-235; rows 1-236; rows 1-237; rows 1-238; rows 1-239; rows 1-241; rows 1-242; rows 1-243; rows 1-244; rows 1-245; rows 1-247; rows 1-248; rows 1-249; rows 1-250; rows 1-252; rows 1-255; rows 1-256; rows 1-257; rows 1-258; rows 1-259; rows 1-264.

As is further shown in Table 4 (Example 4) other groups of genes can predict the prognosis of RRMS patients with 90-94.5% accuracy (average error of “0.1-0.055”).

According to an embodiment of the invention, the polynucleotide sequences which expression level are determined in the cell of the subject diagnosed with MS are those depicted in any of the following groups of row numbers of Table 4 in Example 4 of the Examples section which follows: rows 1-240; rows 1-246; rows 1-251; rows 1-263; rows 1-254; rows 1-260; rows 1-261; rows 1-262; rows 1-263; rows 1-265; rows 1-266; rows 1-267; rows 1-268; rows 1-269; rows 1-270; rows 1-271; rows 1-272; rows 1-273; rows 1-274; rows 1-275; rows 1-276; rows 1-277; rows 1-278; rows 1-279; rows 1-280; rows 1-281; rows 1-282; rows 1-283; rows 1-284; rows 1-285; rows 1-286; rows 1-287; rows 1-288; rows 1-289; rows 1-290; rows 1-291; rows 1-292; rows 1-293; rows 1-294; rows 1-295; rows 1-296; rows 1-297; rows 1-298; rows 1-299; rows 1-300; rows 1-301; rows 1-302; rows 1-3030; rows 1-304; rows 1-305; rows 1-306; rows 1-307; rows 1-308; rows 1-309; rows 1-312; rows 1-313; rows 1-314; rows 1-315; rows 1-316; rows 1-317; rows 1-318; rows 1-324; rows 1-325; rows 1-327; rows 1-328; rows 1-335; rows 1-344;

As used herein the phrase “an alteration above a predetermined threshold” refers to a fold increase or decrease (i.e., degree of upregulation or downregulation, respectively) which is higher than a predetermined threshold such as at least about 1.004, at least about twice, at least about three times, at least about four time, at least about five times, at least about six times, at least about seven times, at least about eight times, at least about nine times, at least about 20 times, at least about 50 times, at least about 100 times, at least about 200 times, at least about 350, at least about 500 times, at least about 1000 times, at least about 2000 times, at least about 3000 times relative to the reference cell.

For example, as is shown in Table 2, while the level of expression of the polynucleotide sequences set forth by SEQ ID NOs:43-136, is at least twice higher in MS patients having good clinical outcome as compared to MS patients having poor clinical outcome, the level of expression of the polynucleotide sequences set forth by SEQ ID NOs:137-161, the polynucleotide sequences set forth by SEQ ID NOs:162-185, the polynucleotide sequences set forth by SEQ ID NOs:186-191 or the polynucleotides set forth by SEQ ID NOs:192-193 is at least 5, 10, 50 or 350 or 150 times, respectively, higher in cells of MS patients having good clinical outcome as compared to cells of MS patients having poor clinical outcome.

In addition, as is further shown in Table 2, while the level of expression of the polynucleotide sequences set forth by SEQ ID NOs:271-366, is at least twice higher in cells of MS patients having poor clinical outcome as compared to cells of MS patients having good clinical outcome, the level of expression of the polynucleotide sequences set forth by SEQ ID NOs:367-399, the polynucleotides set forth by SEQ ID NOs:400-426, the polynucleotides set forth by SEQ ID NOs:427-430 or the polynucleotide set forth by SEQ ID NO:431 is at least 5, 10, 50 or 350 times, respectively, higher in cells of MS patients having poor clinical outcome as compared to cells of MS patients having good clinical outcome.

Thus, the method of predicting the prognosis of a subject diagnosed with MS according to the invention enables the classification of MS patients to those having good prognosis (good clinical outcome, e.g., that will not deteriorate in their neurological disability and that will not experience any relapse for at least 2 years) and those having poor prognosis [poor clinical outcome, e.g., that will deteriorate in their neurological disability (e.g., at least 0.5 point in the EDSS score), with or without relapses)].

It will be appreciated that prediction of the prognosis of a subject diagnosed with MS can be used to select the treatment regimen of a subject and thereby treat the subject diagnosed with MS.

Thus, according to yet another aspect of the invention there is provided a method of treating of a subject diagnosed with multiple sclerosis. The method is effected by: (a) determining in a cell of the subject a level of expression of at least one polynucleotide sequence selected from the group consisting of SEQ ID NOs:1-431, wherein an alteration above a predetermined threshold in the level of expression of the at least one polynucleotide sequence in the cell of the subject relative to a level of expression of the at least one polynucleotide sequence in a reference cell is indicative of a prognosis of the subject diagnosed with multiple sclerosis, and (b) selecting a treatment regimen based on the prognosis, thereby treating the subject diagnosed with multiple sclerosis.

As used herein the phrase “treating” refers to inhibiting or arresting the development of a pathology (multiple sclerosis, e.g., RRMS) and/or causing the reduction, remission, or regression of a pathology and/or optimally curing the pathology. Those of skill in the art will understand that various methodologies and assays can be used to assess the development of the pathology, and similarly, various methodologies and assays may be used to assess the reduction, remission or regression of the pathology.

As used herein the phrase “treatment regimen” refers to a treatment plan that specifies the type of treatment, dosage, schedule and/or duration of a treatment provided to a subject in need thereof (i.e., a subject diagnosed with multiple sclerosis). The selected treatment regimen can be an aggressive one which is expected to result in the best clinical outcome (e.g., complete cure of the pathology), yet may be associated with some discomfort to the subject or adverse side effects (e.g., a damage to healthy cells or tissue); or a more moderate one which may relief symptoms of the pathology yet may results in incomplete cure of the pathology. The type of treatment, dosage, schedule and duration of treatment can vary, depending on the severity of pathology and the predicted outcome (prognosis) of the subject, and those of skills in the art are capable of adjusting the type of treatment with the dosage, schedule and duration of treatment.

According to an embodiment of the invention, when the predicted prognosis of the subject diagnosed with MS is poor prognosis, i.e., there is a high probability that the subject will display an increase of at least 0.5 point in the EDSS score within a period of two years, the treatment regimen selected for treating such a subject according to the method of this aspect of the invention comprises an aggressive therapy using a medicament such as high dosage of interferon beta 1a [Rebif, which can be administered subcutaneously, at a dosage of e.g., 44 μg, three times a week].

According to an embodiment of the invention, when the predicted prognosis of the subject diagnosed with MS is good prognosis, i.e., there is a high probability that the subject will display no change in the EDSS score and no relapses within a period of two years, the treatment regimen selected for treating such a subject according to the method of this aspect of the invention comprises a moderate therapy using a medicament such as moderate dosage of interferon beta 1a [Rebif, which can be administered subcutaneously, at a dosage of e.g., 22 μg, three times a week].

Thus, the teachings of the invention can be used to adapt a treatment regimen to the subject diagnosed with MS according to its predicted clinical outcome as determined with high accuracy (over 89%) by the method of the invention. It will be appreciated that selection of suitable treatment regimens is crucial for achieving cure and remission of symptoms in the affected subjects without exposing them to un-necessary medicaments and on the other hand, is highly beneficial in terms of saving un-necessary costs to the health system.

It will be appreciated that the reagents utilized by any of the methods of the invention which are described hereinabove can form a part of a diagnostic kit/article of manufacture.

The kit of the invention comprises at least 2 and no more than 700 isolated nucleic acid sequences, preferably, at least 4 and no more than 700 isolated nucleic acid sequences, preferably, at least 4 and no more than 600 isolated nucleic acid sequences, preferably, at least 6 and no more than 500 isolated nucleic acid sequences, preferably, at least 6 and no more than 431 isolated nucleic acid sequences, preferably, at least 6 and no more than 34 isolated nucleic acid sequences, wherein each of the at least 2 and no more than 700 isolated nucleic acid sequences is capable of specifically recognizing at least one specific polynucleotide sequence selected from the group consisting of SEQ ID NOs:1-431.

The isolated nucleic acid sequences included in the kit of the invention can be single-stranded or double-stranded, naturally occurring or synthetic nucleic acid sequences such as oligonucleotides, RNA molecules, genomic DNA molecules, cDNA molecules and/or cRNA molecules. The isolated nucleic acid sequences of the kit can be composed of naturally occurring bases, sugars, and covalent internucleoside linkages (e.g., backbone), as well as non-naturally occurring portions, which function similarly to respective naturally occurring portions.

Synthesis of the isolated nucleic acid sequences of the kit can be performed using enzymatic synthesis or solid-phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

According to an embodiment of the invention, each of the isolated nucleic acid sequences included in the kit of invention comprises at least 10 and no more than 50 nucleic acids, more preferably, at least 15 and no more than 45, more preferably, between 15-40, more preferably, between 20-35, more preferably, between 20-30, even more preferably, between 20-25 nucleic acids.

The kit may include at least one reagent as described hereinabove which is suitable for recognizing the at least one specific polynucleotide sequence selected from the group consisting of SEQ ID NOs:1-431. Examples include reagents suitable for hybridization or annealing of a specific polynucleotide of the kit to a specific target polynucleotide sequence (e.g., RNA transcript derived from the cell of the subject or a cDNA derived therefrom) such as formamide, sodium chloride, and sodium citrate), reagents which can be used to labele polynucleotides (e.g., radiolabeled nucleotides, biotinylated nucleotides, digoxigenin-conjugated nucleotides, fluorescent-conjugated nucleotides) as well as reagents suitable for detecting the labeled polynucleotides (e.g., antibodies conjugated to fluorescent dyes, antibodies conjugated to enzymes, radiolabeled antibodies and the like).

Additionally or alternatively, the kit of the invention comprises at least one reagent suitable for detecting the expression level and/or activity of at least one polypeptide encoded by at least one polynucleotides selected from the group consisting of SEQ ID NOs:1-431. Such a reagent can be, for example, an antibody capable of specifically binding to at least one epitope of the polypeptide. Additionally or alternatively, the reagent included in the kit can be a specific substrate capable of binding to an active site of the polypeptide. In addition, the kit may also include reagents such as fluorescent conjugates, secondary antibodies and the like which are suitable for detecting the binding of a specific antibody and/or a specific substrate to the polypeptide.

The kit preferably includes a reference cell which comprises a cell of a subject diagnosed with MS and with a known clinical outcome for at least 24 months as described hereinabove.

The kit of the invention preferably includes packaging material packaging the at least one reagent and a notification in or on the packaging material. Such a notification identifies the kit for use in predicting the prognosis of a subject diagnosed with MS and selecting a treatment regimen of a subject and thereby treating the subject diagnosed with MS. The kit may also include instructions for use in predicting the prognosis of a subject diagnosed with MS and/or selecting a treatment regimen of a subject and/or treating the subject diagnosed with MS. The kit may also include appropriate buffers and preservatives for improving the shelf-life of the kit.

It will be appreciated that the isolated nucleic acid sequences described hereinabove (e.g., oligonucleotides) can form a part of a probeset. The probeset comprises a plurality of oligonucleotides and no more than 700 oligonucleotides wherein each of the plurality of oligonucleotides is capable of specifically recognizing at least one polynucleotide sequence selected from the group consisting of SEQ ID NOs:1-431.

It will be appreciated that the isolated nucleic acid sequences included in the kit or the probeset of the invention can be bound to a solid support e.g., a glass wafer in a specific order, i.e., in the form of an addressable microarray. Alternatively, isolated nucleic acid sequences can be synthesized directly on the solid support using well known prior art approaches (Seo T S, et al., 2004, Proc. Natl. Acad. Sci. USA, 101: 5488-93.). In any case, the isolated nucleic acid sequences are attached to the support in a location specific manner such that each specific isolated nucleic acid sequence has a specific address on the support (i.e., an addressable location) which denotes the identity (i.e., the sequence) of that specific isolated nucleic acid sequence.

According to an embodiment of the invention the microarray comprises no more than 700 isolated nucleic acid sequences, wherein each of the isolated nucleic acid sequences is capable of specifically recognizing at least one specific polynucleotide sequence selected from the group consisting of SEQ ID NOs:1-431.

As used herein the term “about” refers to ±10%.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials, Experimental and Statistical Methods

Study Subjects—

Fifty-three patients with definite relapsing-remitting multiple sclerosis (RRMS) (37 females, 16 males), age 40.2+5.8 years, disease duration 9.9+4.2 years, annual relapse rate 1.3±0.7 and neurological disability evaluated by the Expanded Disability Status Scale (EDSS) (7) 2.0±1.0, were included in the study; 26 patients participated in the differentiating clinical outcome analysis and 27 patients in the validation process of prediction. The clinical and demographic variables were similar between groups and are presented in Table 1, hereinbelow. In the differentiating clinical outcome group 13 patients were on immunomodulatory treatments for at least three months prior to the gene expression study, and 13 patients were naïve to immunomodulatory treatment. In the validation group 11 patients were on immunomodulatory treatments for at least three months prior to the gene expression study, and 16 patients were naïve to immunomodulatory treatments. Within up to one month from blood withdrawing all patients were treated with interferon beta 1-a. None of the patients had ever received cytotoxic treatments and all were free of steroid treatment for at least 30 days before blood was withdrawn. All patients had peripheral blood counts within the normal range. The study was approved by the Sheba Medical Center Institutional Review Board, and all patients gave a written informed consent for participation.

TABLE 1
Clinical characteristics of patients with relapsing-remitting multiple
sclerosis (RRMS)

| | Differentiating clinical | Validation Group |
|---|---|---|
| Characteristic | outcome Group; N = 26 | N = 27 |
| Age (yr) | 40.2 ± 5.8 | 40.5 ± 1.6 |
| F (M) | 21 (5) | 16 (11) |
| Disease duration (yr) | 9.9 ± 4.2 | 10.3 ± 1.6 |
| Relapse rate | 1.3 ± 0.7 | 0.9 ± 0.2 |
| EDSS | 2.0 ± 1.0 | 2.5 ± 0.2 |
| Treated | 13 | 11 |

Table 1 depicts the clinical characteristics of patients with relapsing-remitting multiple sclerosis: patients participated in the differentiating clinical outcome group or in the validation group.
Yr—year;
F—female;
M—male.

Clinical Follow-Up—

Patients were prospectively followed-up for a period of two years. Neurological examination was performed once every three months and at the time of a suspected relapse, and EDSS assessment was completed accordingly. Relapse was defined as the onset of new objective neurological symptoms/signs or worsening of existing neurological disability, not accompanied by metabolic changes, fever or other signs of infection, lasting for a period of at least 48 hours accompanied by objective change of at least 0.5 point in the EDSS score. For EDSS evaluations, only stable EDSS scores that were confirmed at three months follow-up examinations were used. Confirmed relapses and EDSS scores were consecutively recorded.

Definition of Clinical Outcome—

Clinical outcome was defined according to neurological disability as the primary criterion and total number of relapses as the secondary criterion.

Good Outcome:

patients that had not deteriorated in their neurological disability and had not experienced any relapse during the 24 months of follow-up.

Poor Outcome:

Patients that deteriorated in their neurological disability (EDSS increased by at least 0.5 points) within the 24 months of follow-up, either with or without relapses.

Intermediate outcome: Patients that did not deteriorate in their neurological disability yet experienced at least one relapse during the 24 months follow-up.

RNA Isolation and Microarray Expression Profiling—

Peripheral blood mononuclear cells (PBMC) were separated on Ficoll hypaque gradient, total RNA was purified, labeled, hybridized to a Genechip array (U95Av2 and HU-133A) and scanned (Hewlett Packard, GeneArray-TM scanner G2500A) according to the manufacturer's protocol (Affymetrix Inc, Santa Clara, Calif.), as previously described (6).

Clinical Outcome Differentiating Genes Analysis—

RMAExpress software was used to analyze the scanned arrays (8). In order to be consistent with the ontology and array type, all the transcripts in U95Av2 microarray were converted to the corresponding transcripts in HU-133A using NetAffex comparison table. Probesets that did not have a present signal in at least 90% of the samples were filtered. Noise effect was reduced by fitting a multiple effect model for each gene modeling the log-ratio measurement as a sum of contributions for age, gender, batch, subject state (naive or treated), and time from last steroid treatment.

Statistical Methods—

Statistical analysis was performed using the ScoreGenes software tools (http://compbio.cs.huji.ac.il/scoregenes/). Data was analyzed by t-test, threshold number of misclassifications (TNoM) method and the Info-test score. Differentiating genes were defined as genes whose expression was significantly higher or lower with p<0.05 in all three statistical tests. Overabundance analysis was used to compare between the number of observed and expected genes that differentiated between the good and poor clinical outcome under the null hypothesis that the classification of the samples was random (9, 10). To further verify the accuracy of the classification the leave-one-out-cross-validation (LOOCV) statistical method (11) was used. LOOCV simulates removal of a single sample for every trial and trains on the rest. The procedure is repeated until each sample is left out once and the number of correct and incorrect predictions is counted.

Predictive Genes Analysis—

To depict the predictive genes from the differentiating clinical outcome signature support vector machine (SVM) in combination with Forward feature selection algorithm were applied (http://ro(dot)utia(dot)cz/fs/fs_algorithms(dot)html), (12, 13). SVM generates a classifier based on a known labeled training set (19/26 RRMS patients with good or poor clinical outcome from the differentiating clinical outcome group). Then, the classification power of the generated classifier is evaluated by applying it to an independent test set (9/27 RRMS patients from the validation group). The feature selection algorithm finds a subset of predictive genes that enables the generated classifier to achieve the highest classification rate (14, 15). To validate the power of the predictive genes, the classifier was applied to an additional independent set (18/27 RRMS patients from the validation group). The study design is depicted in FIG. 1.

Biological Functional Analysis—

Functional annotation of the clinical outcome differentiating and predictive gene signatures was done using Functional Classification Tools (FCT, David Bioinformatics Resources, http://david(dot)abcc(dot)ncifcrf(dot)gov/home(dot)jsp).

Gene enrichment was defined as group of genes highly associated with a specific biological function and statistically measured by one-tail Fisher Exact Probability Value in the David system. Biological regulatory pathways reconstruction for the predictive gene signature was performed by the (1) PathwayArchitect software http://www(dot)stratagene(dot)com based on literature published data, and the (2) Genomica software http://genomica(dot)weizmann(dot)ac(dot)il that is based on Bayesian networks methods taken from the field of machine learning and was applied to the results of the differentiating gene microarray expression signature. This evaluation was aimed to identify potentially target genes that share a common regulatory mechanism.

Computation of the Average Error in Predicting Clinical Outcome (Good or Poor Prognosis) for Each of the Differentiating Genes—

For each of the 431 differentiating genes (SEQ ID NOs:1-431) the sample was randomly divided into 80% as a “training set” and 20% as a “test set”. The SVM used RBF (radial basic function) kernel to build a model based on the “training set”, which was further tested on the “test set” while saving the error rate. This procedure was repeated 50 times for each gene and the average error for each gene was calculated. Genes with the lowest average error were selected. Then, for each selected gene, the remaining genes were added one after the other, by selecting the next gene such that the average error after 50 repeats of the group of genes including the new gene has the lowest average error as compared to the addition of another gene. This process was repeated 430 times for each additional genes added to the previous group of genes. The results are shown in Table 4, hereinbelow and in FIG. 12.

### Example 1

### Identification of Genetic Markers which are Differentially Expressed Between Patients with Good or Poor Clinical Outcome of RRMS

Experimental and Statistical Results

Clinical Classification of Study Patients—

Patients were classified into three groups based on their clinical disease outcome. Patients with good outcome (N=9, mean age 39.3±3.3 years, disease duration 10.7±3.4 years), patients with intermediate outcome (N=7, mean age 35.8±5.4 years, disease duration 2.6±0.7 years) and patients with poor outcome (N=10, mean age 46.3±4.2 years, disease duration 10.3±0.9). Comparison between outcome variables demonstrated significant difference between patients with good and poor clinical outcome. Change in neurological disability assessed by the EDSS was −0.33±0.24 (good outcome) and 1.6±0.35 (poor outcome), p=0.0002, total number of relapses was 0 (good outcome) and 1.80±0.35 (poor outcome), p=0.00009, respectively.

Differentiating Clinical Outcome Gene Expression Signature—

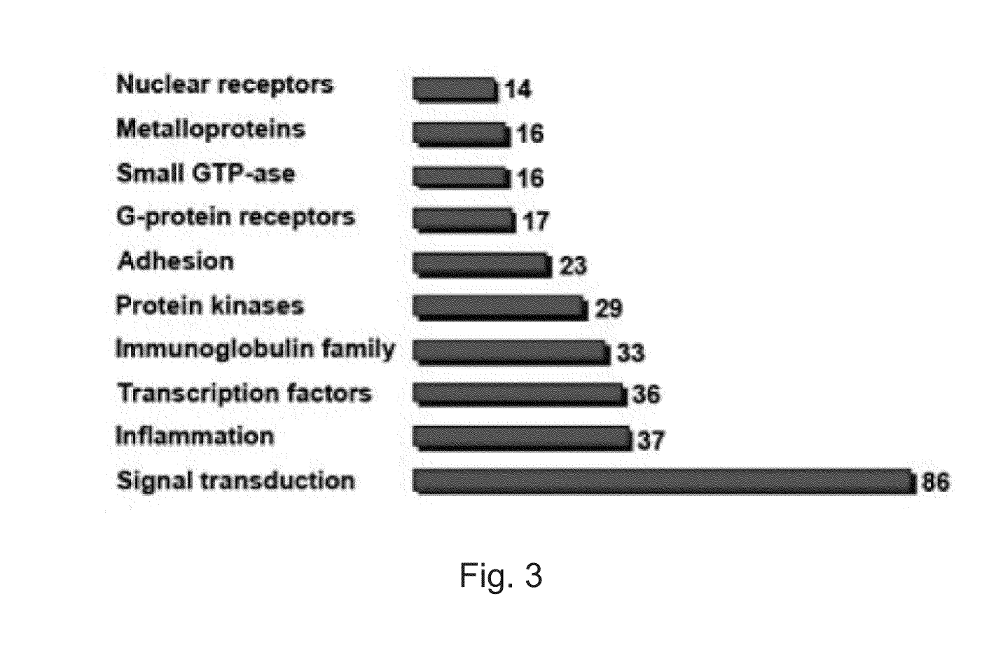

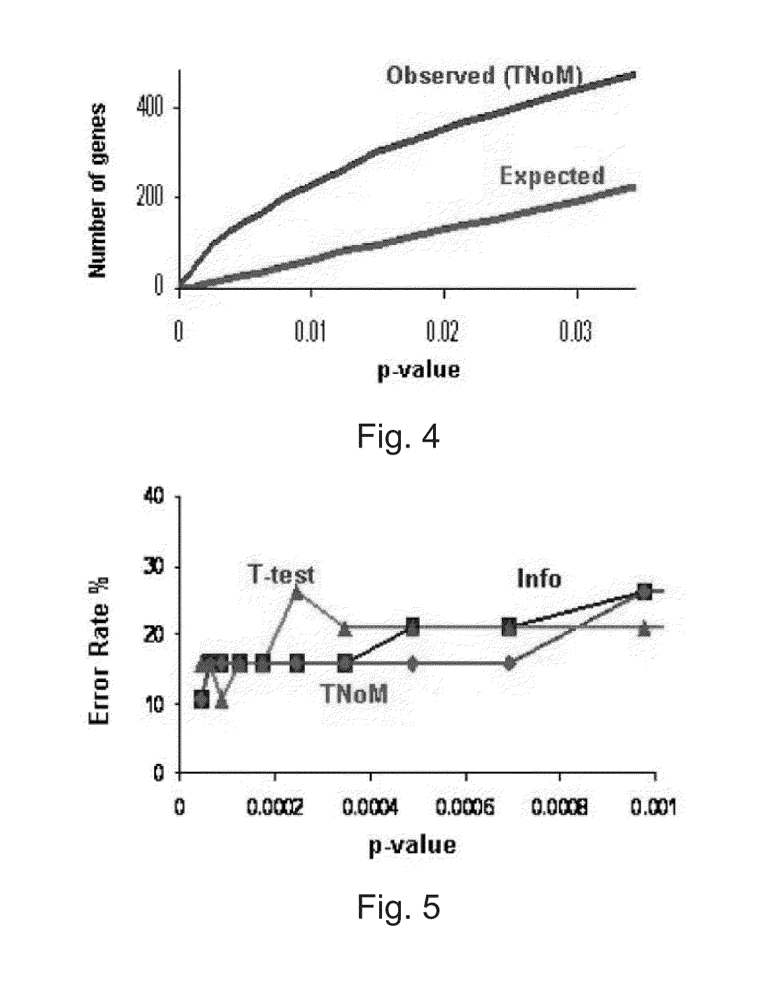

The distinctive clinical outcome gene expression pattern between patients with good and poor clinical outcome included 431 differentiating genes which passed the three statistical tests with p<0.05 (FIG. 2). Functional analysis disclosed genes associated with signal transduction, catalytic activity, adhesion and inflammation (FIG. 3). Overabundance analysis of the observed compared with the expected number of genes that significantly distinguished between patients with good or poor clinical outcome was higher than expected (431 vs 200 genes at p=0.03) (FIG. 4). LOOCV resulted in a high classification rate of 90% p<0.0001 (FIG. 5), suggesting that the differentiating genes signature is reliable and not related to spurious differences due to multiple testing.

TABLE 2
Genetic markers which are differentially expressed between multiple sclerosis patients having
good or poor clinical outcome are provided (the Probeset ID of the Affymetrix Gene Chip), along with
the corresponding GenBank accession number (GenBank Acc. No.), the gene symbol, the SEQ ID NO.,
the p values using the TNOM, Info and t-Test statistical tests, the direction of change in gene expression
(“1”-upregulation; “−1”-downregulation) and the fold change (F/C) in MS patients having poor
clinical outcome as compared to good clinical outcome (Poor/Good). NA—not available.
Clinical outcome differentiating genes in RRMS

| | | SEQ | | | | | F/C | |
|---|---|---|---|---|---|---|---|---|
| Probeset | GenBank | ID | TNOM | Info | t-Test | | (Poor/ | Gene |
| ID | Acc. No. | NO: | PValue | PValue | PValue | Dir | Good) | Symbol |
| 207160_at | NM_000882 | 1 | 2.10E−02 | 3.03E−02 | 3.51E−03 | −1 | 1.004 | IL12A |
| 205034_at | NM_004702 | 2 | 2.10E−02 | 2.60E−02 | 2.71E−03 | −1 | 1.027 | CCNE2 |
| 215935_at | AL080148 | 3 | 4.11E−04 | 2.17E−04 | 3.36E−03 | −1 | 1.055 | C9orf36 |
| 204919_at | NM_007244 | 4 | 2.10E−02 | 3.03E−02 | 3.45E−02 | −1 | 1.108 | PRR4 |
| 201783_s_at | NM_021975 | 5 | 4.11E−04 | 2.17E−04 | 6.77E−05 | −1 | 1.161 | RELA |
| 213457_at | BF739959 | 6 | 3.70E−03 | 3.70E−03 | 1.79E−03 | −1 | 1.368 | MFHAS1 |
| 203145_at | NM_006461 | 7 | 3.70E−03 | 2.49E−03 | 2.82E−03 | −1 | 1.388 | SPAG5 |
| 210822_at | U72513 | 8 | 3.70E−03 | 1.36E−03 | 4.97E−04 | −1 | 1.399 | LOC283345 |
| 206376_at | NM_018057 | 9 | 2.10E−02 | 3.03E−02 | 4.24E−02 | −1 | 1.412 | SLC6A15 |
| 203426_s_at | M65062 | 10 | 2.10E−02 | 2.17E−02 | 2.08E−03 | −1 | 1.439 | IGFBP5 |
| 203205_at | NM_014663 | 11 | 3.70E−03 | 1.36E−03 | 2.65E−03 | −1 | 1.513 | JMJD2A |
| 203324_s_at | NM_001233 | 12 | 2.10E−02 | 2.60E−02 | 1.39E−02 | −1 | 1.516 | CAV2 |
| 207509_s_at | NM_002288 | 13 | 2.10E−02 | 2.60E−02 | 2.90E−02 | −1 | 1.544 | LAIR2 |
| 217165_x_at | M10943 | 14 | 2.10E−02 | 3.03E−02 | 8.33E−03 | −1 | 1.564 | MT1F |
| 215175_at | AB023212 | 15 | 2.10E−02 | 3.03E−02 | 6.57E−03 | −1 | 1.583 | PCNX |
| 215778_x_at | AJ006206 | 16 | 2.10E−02 | 2.60E−02 | 2.29E−02 | −1 | 1.591 | HAB1 |
| 216875_x_at | X83412 | 17 | 2.10E−02 | 2.60E−02 | 2.29E−02 | −1 | 1.591 | HAB1 |
| 205962_at | NM_002577 | 18 | 2.10E−02 | 2.60E−02 | 3.35E−02 | −1 | 1.606 | PAK2 |
| 209685_s_at | M13975 | 19 | 2.10E−02 | 7.04E−03 | 3.04E−03 | −1 | 1.659 | PRKCB1 |
| 207504_at | NM_005182 | 20 | 2.10E−02 | 3.03E−02 | 2.69E−02 | −1 | 1.670 | CA7 |
| 213106_at | AI769688 | 21 | 2.10E−02 | 2.60E−02 | 3.44E−02 | −1 | 1.701 | ATP8A1 |
| 201889_at | NM_014888 | 22 | 2.10E−02 | 1.14E−02 | 4.56E−03 | −1 | 1.722 | FAM3C |
| 209530_at | U07139 | 23 | 3.70E−03 | 3.70E−03 | 2.01E−03 | −1 | 1.727 | CACNB3 |
| 206910_x_at | NM_005666 | 24 | 2.10E−02 | 1.14E−02 | 2.46E−02 | −1 | 1.736 | CFHL2 |
| 213262_at | AI932370 | 25 | 2.10E−02 | 1.14E−02 | 3.86E−02 | −1 | 1.748 | SACS |
| 204316_at | W19676 | 26 | 3.70E−03 | 1.36E−03 | 2.61E−03 | −1 | 1.769 | RGS10 |
| 209031_at | AL519710 | 27 | 2.10E−02 | 2.60E−02 | 1.08E−02 | −1 | 1.783 | IGSF4 |
| 209453_at | M81768 | 28 | 3.70E−03 | 3.70E−03 | 2.89E−02 | −1 | 1.793 | SLC9A1 |
| 212912_at | AI992251 | 29 | 2.10E−02 | 2.17E−02 | 4.27E−03 | −1 | 1.841 | RPS6KA2 |
| 204066_s_at | NM_014914 | 30 | 2.10E−02 | 7.04E−03 | 6.18E−03 | −1 | 1.846 | CENTG2 |
| 206846_s_at | NM_006044 | 31 | 2.10E−02 | 2.17E−02 | 3.55E−03 | −1 | 1.848 | HDAC6 |
| 216224_s_at | AK024083 | 32 | 2.10E−02 | 2.17E−02 | 3.55E−03 | −1 | 1.848 | HDAC6 |
| 205272_s_at | NM_006250 | 33 | 2.10E−02 | 2.60E−02 | 1.88E−02 | −1 | 1.851 | PRH1, PRH2 |
| 214534_at | NM_005322 | 34 | 2.10E−02 | 3.03E−02 | 1.80E−02 | −1 | 1.856 | HIST1H1B |
| 205498_at | NM_000163 | 35 | 2.10E−02 | 2.60E−02 | 2.75E−02 | −1 | 1.903 | GHR |
| 209156_s_at | AY029208 | 36 | 2.10E−02 | 7.04E−03 | 9.25E−03 | −1 | 1.934 | COL6A2 |
| 212534_at | AU144066 | 37 | 2.10E−02 | 7.04E−03 | 4.94E−03 | −1 | 1.936 | ZNF24 |
| 203022_at | NM_006397 | 38 | 2.10E−02 | 7.04E−03 | 1.89E−03 | −1 | 1.972 | RNASEH2A |
| 203071_at | NM_004636 | 39 | 2.10E−02 | 2.60E−02 | 2.61E−02 | −1 | 1.974 | SEMA3B |
| 212242_at | AL565074 | 40 | 3.70E−03 | 2.49E−03 | 2.51E−04 | −1 | 1.974 | TUBA1 |
| 202732_at | NM_007066 | 41 | 2.10E−02 | 1.14E−02 | 3.93E−03 | −1 | 1.990 | PKIG |
| 205013_s_at | NM_000675 | 42 | 2.10E−02 | 2.60E−02 | 3.20E−02 | −1 | 1.993 | ADORA2A |
| 221792_at | AW118072 | 43 | 2.10E−02 | 7.04E−03 | 1.04E−02 | −1 | 2.037 | RAB6B |
| 208135_at | NM_006481 | 44 | 2.10E−02 | 3.03E−02 | 3.31E−02 | −1 | 2.043 | TCF2 |
| 208059_at | NM_005201 | 45 | 3.70E−03 | 3.70E−03 | 4.63E−02 | −1 | 2.050 | CCR8 |
| 207532_at | NM_006891 | 46 | 3.70E−03 | 3.70E−03 | 2.66E−02 | −1 | 2.063 | CRYGD |
| 216699_s_at | L10038 | 47 | 2.10E−02 | 7.04E−03 | 5.23E−03 | −1 | 2.069 | KLK1 |
| 202555_s_at | NM_005965 | 48 | 2.10E−02 | 2.60E−02 | 1.97E−03 | −1 | 2.077 | MYLK |
| 203193_at | NM_004451 | 49 | 2.10E−02 | 7.04E−03 | 9.18E−03 | −1 | 2.119 | ESRRA |
| 215766_at | AL096729 | 50 | 2.10E−02 | 7.04E−03 | 2.58E−03 | −1 | 2.122 | GSTA1 |
| 201023_at | NM_005642 | 51 | 3.70E−03 | 2.49E−03 | 1.34E−03 | −1 | 2.126 | TAF7 |
| 204316_at | W19676 | 52 | 2.10E−02 | 2.17E−02 | 1.11E−02 | −1 | 2.136 | RGS10 |
| 204319_s_at | NM_002925 | 53 | 2.10E−02 | 2.17E−02 | 1.11E−02 | −1 | 2.136 | RGS10 |
| 209477_at | BC000738 | 54 | 2.10E−02 | 1.14E−02 | 2.29E−03 | −1 | 2.166 | EMD |
| 216283_s_at | X64116 | 55 | 2.10E−02 | 3.03E−02 | 3.14E−02 | −1 | 2.169 | PVR |
| 202799_at | NM_006012 | 56 | 3.70E−03 | 2.49E−03 | 8.46E−04 | −1 | 2.171 | CLPP |
| 202127_at | AB011108 | 57 | 2.10E−02 | 7.04E−03 | 4.54E−02 | −1 | 2.195 | PRPF4B |
| 200647_x_at | NM_003752 | 58 | 2.10E−02 | 7.04E−03 | 2.87E−02 | −1 | 2.196 | EIF3S8 |
| 210949_s_at | BC000533 | 59 | 2.10E−02 | 7.04E−03 | 2.87E−02 | −1 | 2.196 | EIF3S8 |
| 215230_x_at | AA679705 | 60 | 2.10E−02 | 7.04E−03 | 2.87E−02 | −1 | 2.196 | EIF3S8 |
| 210328_at | AF101477 | 61 | 2.10E−02 | 2.60E−02 | 3.52E−02 | −1 | 2.260 | GNMT |
| 211988_at | BG289800 | 62 | 4.11E−04 | 2.17E−04 | 2.74E−04 | −1 | 2.265 | SMARCE1 |
| 215781_s_at | D87012 | 63 | 2.10E−02 | 2.60E−02 | 2.98E−02 | −1 | 2.273 | TOP3B |
| 204163_at | NM_007046 | 64 | 2.10E−02 | 3.03E−02 | 2.63E−02 | −1 | 2.319 | EMILIN1 |
| 207954_at | NM_002050 | 65 | 3.70E−03 | 3.70E−03 | 1.50E−03 | −1 | 2.323 | GATA2 |
| 210358_x_at | BC002557 | 66 | 3.70E−03 | 3.70E−03 | 1.50E−03 | −1 | 2.323 | GATA2 |
| 222206_s_at | AA781143 | 67 | 2.10E−02 | 7.04E−03 | 8.50E−03 | −1 | 2.357 | NICALIN |
| 200949_x_at | NM_001023 | 68 | 2.10E−02 | 1.14E−02 | 4.42E−02 | −1 | 2.368 | RPS20 |
| 214003_x_at | BF184532 | 69 | 2.10E−02 | 1.14E−02 | 4.42E−02 | −1 | 2.368 | RPS20 |
| 203701_s_at | NM_017722 | 70 | 2.10E−02 | 1.14E−02 | 8.29E−03 | −1 | 2.375 | FLJ20244 |
| 210463_x_at | BC002492 | 71 | 2.10E−02 | 1.14E−02 | 8.29E−03 | −1 | 2.375 | FLJ20244 |
| 204703_at | NM_006531 | 72 | 2.10E−02 | 7.04E−03 | 1.46E−02 | −1 | 2.398 | TTC10 |
| 216582_at | AL021808 | 73 | 2.10E−02 | 2.60E−02 | 6.09E−03 | −1 | 2.451 | AL021808 |
| 208687_x_at | AF352832 | 74 | 3.70E−03 | 3.70E−03 | 3.18E−02 | −1 | 2.472 | HSPA8 |
| 202314_at | NM_000786 | 75 | 2.10E−02 | 2.60E−02 | 1.22E−02 | −1 | 2.509 | CYP51A1 |
| 214095_at | AW190316 | 76 | 2.10E−02 | 2.60E−02 | 3.91E−03 | −1 | 2.535 | SHMT2 |
| 214096_s_at | AW190316 | 77 | 2.10E−02 | 2.60E−02 | 3.91E−03 | −1 | 2.535 | SHMT2 |
| 214948_s_at | AL050136 | 78 | 2.10E−02 | 2.17E−02 | 4.68E−03 | −1 | 2.568 | TMF1 |
| 206879_s_at | NM_013982 | 79 | 2.10E−02 | 7.04E−03 | 1.57E−02 | −1 | 2.618 | NRG2 |
| 205463_s_at | NM_002607 | 80 | 3.70E−03 | 3.70E−03 | 7.59E−04 | −1 | 2.639 | PDGFA |
| 205022_s_at | NM_005197 | 81 | 3.70E−03 | 2.49E−03 | 8.78E−03 | −1 | 2.655 | CHES1 |
| 203801_at | BG254653 | 82 | 2.10E−02 | 7.04E−03 | 8.11E−03 | −1 | 2.671 | MRPS14 |
| 215746_at | L34409 | 83 | 2.10E−02 | 3.03E−02 | 1.80E−02 | −1 | 2.676 | TETRAN |
| 201418_s_at | NM_003107 | 84 | 3.70E−03 | 1.36E−03 | 1.33E−03 | −1 | 2.721 | SOX4 |
| 202410_x_at | NM_000612 | 85 | 3.70E−03 | 1.36E−03 | 1.21E−02 | −1 | 2.795 | IGF2 |
| 204400_at | NM_005864 | 86 | 3.70E−03 | 1.36E−03 | 6.24E−04 | −1 | 2.815 | EFS |
| 210880_s_at | AB001467 | 87 | 3.70E−03 | 1.36E−03 | 6.24E−04 | −1 | 2.815 | EFS |
| 200790_at | NM_002539 | 88 | 2.10E−02 | 2.60E−02 | 6.27E−03 | −1 | 2.817 | ODC1 |
| 203339_at | AI887457 | 89 | 2.10E−02 | 7.04E−03 | 1.11E−02 | −1 | 2.853 | SLC25A12 |
| 203340_s_at | AI887457 | 90 | 2.10E−02 | 7.04E−03 | 1.11E−02 | −1 | 2.853 | SLC25A12 |
| 201513_at | NM_004622 | 91 | 2.10E−02 | 3.03E−02 | 1.07E−02 | −1 | 2.915 | TSN |
| 204159_at | NM_001262 | 92 | 2.10E−02 | 7.04E−03 | 2.76E−03 | −1 | 2.920 | CDKN2C |
| 211792_s_at | U17074 | 93 | 2.10E−02 | 7.04E−03 | 2.76E−03 | −1 | 2.920 | CDKN2C |
| 209320_at | AF033861 | 94 | 2.10E−02 | 1.14E−02 | 2.07E−03 | −1 | 2.975 | ADCY3 |
| 209480_at | M16276 | 95 | 4.11E−04 | 2.17E−04 | 2.15E−05 | −1 | 2.983 | MHC II, |
| | | | | | | | | DQ beta 1 |
| 212999_x_at | AW276186 | 96 | 4.11E−04 | 2.17E−04 | 2.15E−05 | −1 | 2.983 | MHC II, |
| | | | | | | | | DQ beta 1 |
| 214613_at | AW024085 | 97 | 2.10E−02 | 7.04E−03 | 1.72E−02 | −1 | 3.021 | GPR3 |
| 201269_s_at | AB028991 | 98 | 2.10E−02 | 2.17E−02 | 3.88E−02 | −1 | 3.142 | KIAA1068 |
| 209361_s_at | BC004153 | 99 | 3.70E−03 | 1.36E−03 | 1.60E−02 | −1 | 3.183 | PCBP4 |
| 213840_s_at | R68573 | 100 | 2.10E−02 | 3.03E−02 | 1.34E−02 | −1 | 3.196 | — |
| 204895_x_at | NM_004532 | 101 | 3.70E−03 | 3.70E−03 | 2.34E−02 | −1 | 3.206 | MUC4 |
| 217109_at | AJ242547 | 102 | 3.70E−03 | 3.70E−03 | 2.34E−02 | −1 | 3.206 | MUC4 |
| 217110_s_at | AJ242547 | 103 | 3.70E−03 | 3.70E−03 | 2.34E−02 | −1 | 3.206 | MUC4 |
| 212852_s_at | AL538601 | 104 | 3.70E−03 | 1.36E−03 | 1.29E−03 | −1 | 3.225 | SSA2 |
| 206759_at | NM_002002 | 105 | 2.10E−02 | 1.14E−02 | 5.20E−03 | −1 | 3.227 | FCER2 |
| 206760_s_at | NM_002002 | 106 | 2.10E−02 | 1.14E−02 | 5.20E−03 | −1 | 3.227 | FCER2 |
| 203422_at | NM_002691 | 107 | 2.10E−02 | 1.14E−02 | 1.86E−02 | −1 | 3.245 | POLD1 |
| 202766_s_at | NM_000138 | 108 | 3.70E−03 | 3.70E−03 | 2.33E−02 | −1 | 3.303 | FBN1 |
| 216065_at | AL031228 | 109 | 2.10E−02 | 3.03E−02 | 2.88E−02 | −1 | 3.528 | BING5 |
| 202140_s_at | NM_003992 | 110 | 2.10E−02 | 1.14E−02 | 1.85E−03 | −1 | 3.579 | CLK3 |
| 209107_x_at | U19179 | 111 | 3.70E−03 | 1.36E−03 | 8.37E−04 | −1 | 3.612 | NCOA1 |
| 210249_s_at | U59302 | 112 | 3.70E−03 | 1.36E−03 | 8.37E−04 | −1 | 3.612 | NCOA1 |
| 208041_at | NM_002929 | 113 | 3.70E−03 | 1.36E−03 | 5.34E−03 | −1 | 3.659 | GRK1 |
| 202554_s_at | AL527430 | 114 | 2.10E−02 | 2.60E−02 | 1.26E−02 | −1 | 3.775 | GSTM3 |
| 213815_x_at | AI913329 | 115 | 2.10E−02 | 2.60E−02 | 3.04E−03 | −1 | 3.820 | NY-REN 24 |
| 214892_x_at | BC004262 | 116 | 2.10E−02 | 2.60E−02 | 3.04E−03 | −1 | 3.820 | C19orf29 |
| 215954_s_at | AI200896 | 117 | 2.10E−02 | 2.60E−02 | 3.04E−03 | −1 | 3.820 | NY-REN 24 |
| 204144_s_at | NM_004204 | 118 | 3.70E−03 | 1.36E−03 | 9.11E−04 | −1 | 3.873 | PIGQ |
| 206592_s_at | NM_003938 | 119 | 3.70E−03 | 3.70E−03 | 3.71E−03 | −1 | 3.881 | AP3D1 |
| 200936_at | NM_000973 | 120 | 3.70E−03 | 2.49E−03 | 1.59E−03 | −1 | 3.915 | RPL8 |
| 204065_at | NM_004854 | 121 | 3.70E−03 | 1.36E−03 | 6.52E−03 | −1 | 4.113 | CHST10 |
| 206327_s_at | NM_004933 | 122 | 2.10E−02 | 2.17E−02 | 1.74E−02 | −1 | 4.164 | CDH15 |
| 206328_at | NM_004933 | 123 | 2.10E−02 | 2.17E−02 | 1.74E−02 | −1 | 4.164 | CDH15 |
| 205608_s_at | U83508 | 124 | 3.70E−03 | 2.49E−03 | 1.47E−03 | −1 | 4.205 | ANGPT1 |
| 204197_s_at | NM_004350 | 125 | 2.10E−02 | 3.03E−02 | 2.22E−02 | −1 | 4.271 | RUNX3 |
| 205683_x_at | NM_003294 | 126 | 2.10E−02 | 2.17E−02 | 8.61E−03 | −1 | 4.292 | TPSAB1 |
| 207134_x_at | NM_024164 | 127 | 2.10E−02 | 2.17E−02 | 8.61E−03 | −1 | 4.292 | TPSB2 |
| 216474_x_at | AF206667 | 128 | 2.10E−02 | 2.17E−02 | 8.61E−03 | −1 | 4.292 | TPSAB1, |
| | | | | | | | | TPSB2 |
| 214487_s_at | NM_002886 | 129 | 2.10E−02 | 2.60E−02 | 1.21E−02 | −1 | 4.296 | RAP2A, RAP2B |
| 214488_at | NM_002886 | 130 | 2.10E−02 | 2.60E−02 | 1.21E−02 | −1 | 4.296 | RAP2B |
| 212674_s_at | AK002076 | 131 | 3.70E−03 | 3.70E−03 | 2.98E−02 | −1 | 4.426 | DHX30 |
| 209253_at | AF037261 | 132 | 2.10E−02 | 2.60E−02 | 7.17E−03 | −1 | 4.520 | SCAM-1 |
| 210619_s_at | AF173154 | 133 | 2.10E−02 | 3.03E−02 | 9.84E−03 | −1 | 4.648 | HYAL1 |
| 213221_s_at | AB018324 | 134 | 3.70E−03 | 3.70E−03 | 2.54E−03 | −1 | 4.758 | SIK2 |
| 216939_s_at | Y08756 | 135 | 2.10E−02 | 7.04E−03 | 6.08E−03 | −1 | 4.910 | HTR4, |
| | | | | | | | | KIAA1985 |
| 202415_s_at | NM_012267 | 136 | 2.10E−02 | 7.04E−03 | 1.51E−02 | −1 | 4.922 | HSPBP1 |
| 205798_at | NM_002185 | 137 | 2.10E−02 | 3.03E−02 | 2.08E−02 | −1 | 5.081 | IL7R |
| 207938_at | NM_015886 | 138 | 2.10E−02 | 2.60E−02 | 4.15E−02 | −1 | 5.111 | PI15 |
| 217060_at | U03115 | 139 | 2.10E−02 | 3.03E−02 | 4.35E−02 | −1 | 5.115 | TCRBV |
| 207900_at | NM_002987 | 140 | 2.10E−02 | 1.14E−02 | 6.92E−03 | −1 | 5.125 | CCL17 |
| 205189_s_at | NM_000136 | 141 | 2.10E−02 | 1.14E−02 | 1.89E−02 | −1 | 5.128 | FANCC |
| 208009_s_at | NM_014448 | 142 | 2.10E−02 | 2.17E−02 | 2.24E−02 | −1 | 5.145 | ARHGEF16 |
| 206148_at | NM_002183 | 143 | 2.10E−02 | 3.03E−02 | 2.62E−02 | −1 | 5.170 | IL3RA |
| 204816_s_at | AI924903 | 144 | 2.10E−02 | 3.03E−02 | 8.65E−03 | −1 | 5.215 | DHX34 |
| 210306_at | U89358 | 145 | 3.70E−03 | 3.70E−03 | 7.60E−04 | −1 | 5.331 | L3MBTL |
| 206398_s_at | NM_001770 | 146 | 2.10E−02 | 1.14E−02 | 2.36E−03 | −1 | 5.693 | CD19 |
| 212540_at | BG476661 | 147 | 2.10E−02 | 2.17E−02 | 7.82E−03 | −1 | 5.949 | CDC34 |
| 207694_at | NM_000307 | 148 | 2.10E−02 | 1.14E−02 | 5.97E−03 | −1 | 6.007 | POU3F4 |
| 202268_s_at | NM_003905 | 149 | 2.10E−02 | 2.60E−02 | 1.30E−02 | −1 | 6.029 | APPBP1 |
| 202909_at | NM_014805 | 150 | 2.10E−02 | 3.03E−02 | 1.30E−02 | −1 | 6.919 | EPM2AIP1 |
| 205798_at | NM_002185 | 151 | 2.10E−02 | 3.03E−02 | 1.54E−02 | −1 | 6.948 | IL7R |
| 203421_at | NM_006034 | 152 | 2.10E−02 | 2.60E−02 | 1.35E−02 | −1 | 6.959 | TP53I11 |
| 207403_at | NM_003604 | 153 | 2.10E−02 | 2.60E−02 | 1.79E−03 | −1 | 6.981 | IRS4 |
| 202312_s_at | K01228 | 154 | 2.10E−02 | 3.03E−02 | 3.84E−02 | −1 | 7.027 | COL1Al |
| 201497_x_at | NM_022844 | 155 | 2.10E−02 | 3.03E−02 | 1.40E−02 | −1 | 7.285 | MYH11 |
| 203683_s_at | NM_003377 | 156 | 2.10E−02 | 3.03E−02 | 4.63E−03 | −1 | 7.314 | VEGFB |
| 210438_x_at | M25077 | 157 | 2.10E−02 | 1.14E−02 | 1.40E−02 | −1 | 8.214 | SSA2 |
| 205805_s_at | NM_005012 | 158 | 2.17E−05 | 2.17E−05 | 2.07E−05 | −1 | 8.307 | ROR1 |
| 207222_at | NM_003561 | 159 | 2.10E−02 | 3.03E−02 | 8.61E−03 | −1 | 9.192 | PLA2G10 |
| 203329_at | NM_002845 | 160 | 4.11E−04 | 2.17E−04 | 3.04E−05 | −1 | 9.736 | PTPRM |
| 207036_x_at | NM_000836 | 161 | 2.10E−02 | 1.14E−02 | 1.43E−02 | −1 | 9.843 | GRIN2D |
| 208580_x_at | NM_021968 | 162 | 3.70E−03 | 2.49E−03 | 7.90E−03 | −1 | 10.804 | HIST1H4J, |
| | | | | | | | | HIST1H4K |
| 214463_x_at | NM_003541 | 163 | 3.70E−03 | 2.49E−03 | 7.90E−03 | −1 | 10.804 | HIST1H4J, |
| | | | | | | | | HIST1H4K |
| 208091_s_at | NM_030796 | 164 | 2.10E−02 | 7.04E−03 | 6.78E−03 | −1 | 10.908 | DKFZP564K08 |
| | | | | | | | | 22 |
| 206516_at | NM_000479 | 165 | 4.11E−04 | 4.11E−04 | 8.60E−04 | −1 | 11.814 | AMH |
| 200834_s_at | NM_001024 | 166 | 3.70E−03 | 3.70E−03 | 1.35E−02 | −1 | 11.908 | RPS21 |
| 208105_at | NM_000164 | 167 | 2.10E−02 | 2.60E−02 | 3.55E−03 | −1 | 12.069 | GIPR |
| 207959_s_at | NM_004662 | 168 | 2.10E−02 | 2.60E−02 | 4.54E−02 | −1 | 12.457 | DNAH9 |
| 210345_s_at | AF257737 | 169 | 2.10E−02 | 2.60E−02 | 4.54E−02 | −1 | 12.457 | DNAH9 |
| 202315_s_at | NM_004327 | 170 | 4.11E−04 | 2.17E−04 | 3.36E−06 | −1 | 12.559 | BCR |
| 208733_at | AW301641 | 171 | 3.70E−03 | 1.36E−03 | 6.96E−04 | −1 | 12.569 | RAB2 |
| 221847_at | BF665706 | 172 | 2.10E−02 | 2.60E−02 | 5.69E−03 | −1 | 13.019 | — |
| 214481_at | NM_003514 | 173 | 2.10E−02 | 3.03E−02 | 1.06E−02 | −1 | 13.748 | HIST1H2AM |
| 214644_at | BF061074 | 174 | 3.70E−03 | 2.49E−03 | 1.08E−03 | −1 | 14.563 | HIST1H2AK |
| 217192_s_at | AL022067 | 175 | 2.10E−02 | 1.14E−02 | 7.64E−03 | −1 | 14.756 | PRDM1 |
| 220937_s_at | NM_014403 | 176 | 2.10E−02 | 1.14E−02 | 6.81E−03 | −1 | 15.370 | SIAT7D |
| 221551_x_at | AW044319 | 177 | 2.10E−02 | 1.14E−02 | 6.81E−03 | −1 | 15.370 | SIAT7D |
| 215266_at | AL096732 | 178 | 2.10E−02 | 2.60E−02 | 8.04E−03 | −1 | 17.220 | DNAH3 |
| 203851_at | NM_002178 | 179 | 2.10E−02 | 2.17E−02 | 4.92E−03 | −1 | 17.962 | IGFBP6 |
| 209466_x_at | M57399 | 180 | 2.10E−02 | 7.04E−03 | 6.13E−03 | −1 | 21.339 | PTN |
| 211737_x_at | BC005916 | 181 | 2.10E−02 | 7.04E−03 | 6.13E−03 | −1 | 21.339 | PTN |
| 209686_at | BC001766 | 182 | 2.10E−02 | 3.03E−02 | 8.52E−03 | −1 | 22.433 | S100B |
| 216867_s_at | X03795 | 183 | 2.10E−02 | 2.60E−02 | 1.47E−02 | −1 | 26.553 | PDGFA |
| 210229_s_at | M11734 | 184 | 2.10E−02 | 2.17E−02 | 1.02E−02 | −1 | 36.065 | CSF2 |
| 209651_at | BC001830 | 185 | 2.10E−02 | 3.03E−02 | 6.32E−03 | −1 | 42.216 | TGFB1I1 |
| 206616_s_at | AF155382 | 186 | 4.11E−04 | 4.11E−04 | 8.65E−04 | −1 | 59.650 | ADAM22 |
| 208226_x_at | NM_004194 | 187 | 4.11E−04 | 4.11E−04 | 8.65E−04 | −1 | 59.650 | ADAM22 |
| 208227_x_at | NM_021721 | 188 | 4.11E−04 | 4.11E−04 | 8.65E−04 | −1 | 59.650 | ADAM22 |
| 208237_x_at | NM_021722 | 189 | 4.11E−04 | 4.11E−04 | 8.65E−04 | −1 | 59.650 | ADAM22 |
| 216993_s_at | U32169 | 190 | 2.10E−02 | 2.60E−02 | 1.55E−02 | −1 | 65.430 | COL11A2 |
| 209726_at | AB018195 | 191 | 3.70E−03 | 1.36E−03 | 1.03E−02 | −1 | 74.847 | CA11 |
| 206944_at | AF007141 | 192 | 2.10E−02 | 7.04E−03 | 3.15E−02 | −1 | 382.037 | HTR6 |
| 203503_s_at | NM_004565 | 193 | 2.10E−02 | 3.03E−02 | 4.90E−02 | −1 | NA | PEX14 |
| 214994_at | BF508948 | 194 | 3.70E−03 | 2.49E−03 | 1.12E−03 | 1 | 1.010 | APOBEC3F |
| 202270_at | NM_002053 | 195 | 3.70E−03 | 3.70E−03 | 2.70E−03 | 1 | 1.012 | GBP1 |
| 201975_at | NM_002956 | 196 | 4.11E−04 | 2.17E−04 | 4.98E−04 | 1 | 1.070 | RSN |
| 206584_at | NM_015364 | 197 | 4.11E−04 | 2.17E−04 | 1.05E−02 | 1 | 1.095 | LY96 |
| 212561_at | AA349595 | 198 | 2.10E−02 | 3.03E−02 | 3.47E−02 | 1 | 1.126 | RAB6IP1 |
| 209413_at | BC002431 | 199 | 3.70E−03 | 2.49E−03 | 4.18E−04 | 1 | 1.156 | B4GALT2 |
| 214507_s_at | NM_014285 | 200 | 3.70E−03 | 2.49E−03 | 8.67E−04 | 1 | 1.249 | EXOSC2 |
| 212819_at | AF055024 | 201 | 3.70E−03 | 1.36E−03 | 1.93E−03 | 1 | 1.260 | ASB1 |
| 206364_at | NM_014875 | 202 | 2.10E−02 | 3.03E−02 | 9.68E−03 | 1 | 1.328 | KIF14 |
| 206421_s_at | NM_003784 | 203 | 2.10E−02 | 2.60E−02 | 3.81E−02 | 1 | 1.388 | SERPINB7 |
| 209961_s_at | M60718 | 204 | 2.10E−02 | 2.60E−02 | 4.30E−02 | 1 | 1.389 | HGF |
| 204202_at | NM_017604 | 205 | 2.10E−02 | 7.04E−03 | 8.75E−03 | 1 | 1.413 | IQCE |
| 207872_s_at | NM_006863 | 206 | 3.70E−03 | 2.49E−03 | 6.77E−04 | 1 | 1.434 | LILRA1 |
| 215906_at | 565921 | 207 | 2.10E−02 | 3.03E−02 | 3.41E−02 | 1 | 1.459 | ACCLC |
| 204849_at | NM_006602 | 208 | 3.70E−03 | 3.70E−03 | 2.40E−02 | 1 | 1.506 | TCFLS |
| 211832_s_at | AF201370 | 209 | 2.10E−02 | 7.04E−03 | 1.33E−02 | 1 | 1.527 | MDM2 |
| 207867_at | NM_006193 | 210 | 2.10E−02 | 2.60E−02 | 2.32E−02 | 1 | 1.539 | PAX4 |
| 203501_at | NM_006102 | 211 | 3.70E−03 | 3.70E−03 | 3.39E−03 | 1 | 1.549 | PGCP |
| 208454_s_at | NM_016134 | 212 | 3.70E−03 | 3.70E−03 | 3.39E−03 | 1 | 1.549 | PGCP |
| 206426_at | NM_005511 | 213 | 2.10E−02 | 2.60E−02 | 4.10E−02 | 1 | 1.558 | MLANA |
| 208193_at | NM_000590 | 214 | 2.10E−02 | 2.60E−02 | 2.01E−02 | 1 | 1.561 | IL9 |
| 201538_s_at | AL048503 | 215 | 2.10E−02 | 7.04E−03 | 3.54E−02 | 1 | 1.600 | DUSP3 |
| 214872_at | AL080129 | 216 | 2.10E−02 | 1.14E−02 | 1.73E−03 | 1 | 1.613 | Rif1 |
| 205885_s_at | NM_000885 | 217 | 2.10E−02 | 1.14E−02 | 5.96E−03 | 1 | 1.631 | ITGA4 |
| 205062_x_at | NM_002892 | 218 | 2.10E−02 | 1.14E−02 | 2.08E−02 | 1 | 1.637 | ARID4A |
| 209245_s_at | AB014606 | 219 | 2.10E−02 | 2.60E−02 | 8.48E−03 | 1 | 1.645 | KIF1C |
| 212976_at | R41498 | 220 | 2.10E−02 | 1.14E−02 | 2.83E−02 | 1 | 1.645 | TA-LRRP |
| 200797_s_at | AI275690 | 221 | 3.70E−03 | 3.70E−03 | 4.15E−02 | 1 | 1.650 | MCL1 |
| 203266_s_at | NM_003010 | 222 | 2.10E−02 | 1.14E−02 | 2.07E−02 | 1 | 1.655 | MAP2K4 |
| 208042_at | NM_013303 | 223 | 3.70E−03 | 1.36E−03 | 7.53E−03 | 1 | 1.655 | VG5Q |
| 207037_at | NM_003839 | 224 | 2.10E−02 | 2.17E−02 | 7.68E−03 | 1 | 1.667 | TNFRSF11A |
| 206911_at | NM_005082 | 225 | 2.10E−02 | 7.04E−03 | 2.82E−03 | 1 | 1.671 | TRIM25 |
| 208359_s_at | NM_004981 | 226 | 2.10E−02 | 2.60E−02 | 2.42E−02 | 1 | 1.674 | KCNJ4 |
| 211451_s_at | U24056 | 227 | 2.10E−02 | 2.60E−02 | 2.42E−02 | 1 | 1.674 | KCNJ4 |
| 212882_at | AB018338 | 228 | 2.10E−02 | 2.60E−02 | 1.03E−02 | 1 | 1.676 | KLHL18 |
| 205141_at | NM_001145 | 229 | 2.10E−02 | 7.04E−03 | 9.28E−03 | 1 | 1.713 | ANG |
| 216695_s_at | AF082559 | 230 | 3.70E−03 | 1.36E−03 | 3.13E−03 | 1 | 1.721 | TNKS |
| 205312_at | NM_003120 | 231 | 2.10E−02 | 2.60E−02 | 1.99E−02 | 1 | 1.722 | SPI1 |
| 205990_s_at | NM_003392 | 232 | 2.10E−02 | 3.03E−02 | 3.56E−02 | 1 | 1.746 | WNTSA |
| 202260_s_at | NM_003165 | 233 | 3.70E−03 | 3.70E−03 | 2.60E−03 | 1 | 1.747 | STXBP1 |
| 214624_at | AA548647 | 234 | 3.70E−03 | 1.36E−03 | 1.08E−02 | 1 | 1.750 | UPK1A |
| 211561_x_at | L35253 | 235 | 4.11E−04 | 2.17E−04 | 9.33E−05 | 1 | 1.760 | MAPK14 |
| 216817_s_at | AJ302604 | 236 | 2.10E−02 | 1.14E−02 | 7.08E−03 | 1 | 1.764 | OR2H1 |
| 207044_at | NM_000461 | 237 | 2.10E−02 | 7.04E−03 | 1.51E−02 | 1 | 1.766 | THRB |
| 208724_s_at | BC000905 | 238 | 3.70E−03 | 3.70E−03 | 3.32E−02 | 1 | 1.770 | RAB1A |
| 214111_at | AF070577 | 239 | 2.10E−02 | 7.04E−03 | 4.11E−02 | 1 | 1.774 | AF070577 |
| 205261_at | NM_002630 | 240 | 2.10E−02 | 7.04E−03 | 1.86E−02 | 1 | 1.781 | PGC |
| 207406_at | NM_000780 | 241 | 2.10E−02 | 7.04E−03 | 1.64E−02 | 1 | 1.787 | CYP7A1 |
| 206218_at | NM_002364 | 242 | 2.10E−02 | 7.04E−03 | 9.00E−04 | 1 | 1.791 | MAGEB 2 |
| 207010_at | NM_000812 | 243 | 2.10E−02 | 2.60E−02 | 4.13E−03 | 1 | 1.801 | GABRB1 |
| 217301_x_at | X71810 | 244 | 2.10E−02 | 3.03E−02 | 6.23E−03 | 1 | 1.822 | RBBP4 |
| 211108_s_at | U31601 | 245 | 3.70E−03 | 3.70E−03 | 5.90E−03 | 1 | 1.826 | JAK3 |
| 211109_at | U31601 | 246 | 3.70E−03 | 3.70E−03 | 5.90E−03 | 1 | 1.826 | JAK3 |
| 206902_s_at | NM_005728 | 247 | 3.70E−03 | 1.36E−03 | 1.14E−04 | 1 | 1.853 | ENDOGL1 |
| 206903_at | NM_005728 | 248 | 3.70E−03 | 1.36E−03 | 1.14E−04 | 1 | 1.853 | ENDOGL2 |
| 209260_at | BC000329 | 249 | 2.10E−02 | 3.03E−02 | 1.58E−02 | 1 | 1.879 | SFN |
| 214099_s_at | AK001619 | 250 | 3.70E−03 | 2.49E−03 | 1.54E−02 | 1 | 1.883 | PDE4DIP |
| 201453_x_at | NM_005614 | 251 | 2.10E−02 | 3.03E−02 | 1.52E−02 | 1 | 1.891 | RHEB |
| 213404_s_at | BF033683 | 252 | 2.10E−02 | 3.03E−02 | 1.52E−02 | 1 | 1.891 | RHEB |
| 202805_s_at | NM_004996 | 253 | 4.11E−04 | 4.11E−04 | 6.42E−04 | 1 | 1.892 | ABCC1 |
| 215008_at | AA582404 | 254 | 2.10E−02 | 7.04E−03 | 2.49E−03 | 1 | 1.897 | TLL2 |
| 205619_s_at | NM_004527 | 255 | 2.10E−02 | 3.03E−02 | 1.38E−02 | 1 | 1.905 | MEOX1 |
| 215130_s_at | AC002550 | 256 | 2.10E−02 | 3.03E−02 | 4.39E−02 | 1 | 1.906 | MGC35048 |
| 215131_at | AC002550 | 257 | 2.10E−02 | 3.03E−02 | 4.39E−02 | 1 | 1.906 | MGC35048 |
| 207675_x_at | NM_003976 | 258 | 2.10E−02 | 7.04E−03 | 4.31E−03 | 1 | 1.922 | ARTN |
| 216052_x_at | AF115765 | 259 | 2.10E−02 | 7.04E−03 | 4.31E−03 | 1 | 1.922 | ARTN |
| 205962_at | NM_002577 | 260 | 2.10E−02 | 1.14E−02 | 8.39E−04 | 1 | 1.930 | PAK2 |
| 207143_at | NM_001259 | 261 | 3.70E−03 | 1.36E−03 | 1.29E−02 | 1 | 1.932 | CDK6 |
| 203755_at | NM_001211 | 262 | 4.11E−04 | 2.17E−04 | 5.38E−04 | 1 | 1.941 | BUB1B |
| 202166_s_at | NM_006241 | 263 | 3.70E−03 | 3.70E−03 | 1.74E−02 | 1 | 1.948 | PPP1R2 |
| 206570_s_at | NM_002785 | 264 | 2.10E−02 | 2.17E−02 | 1.17E−02 | 1 | 1.953 | PSG8, PSG4, |
| | | | | | | | | PSG9, PSG3, |
| | | | | | | | | PSG7 |
| 202886_s_at | M65254 | 265 | 2.10E−02 | 7.04E−03 | 5.42E−03 | 1 | 1.959 | PPP2R1B |
| 200889_s_at | NM_003144 | 266 | 3.70E−03 | 2.49E−03 | 4.88E−04 | 1 | 1.965 | SSR1 |
| 206942_s_at | NM_002674 | 267 | 3.70E−03 | 1.36E−03 | 6.48E−03 | 1 | 1.969 | PMCH |
| 207862_at | NM_006760 | 268 | 2.10E−02 | 7.04E−03 | 2.52E−03 | 1 | 1.976 | UPK2 |
| 200771_at | NM_002293 | 269 | 2.10E−02 | 1.14E−02 | 3.76E−03 | 1 | 1.978 | LAMC1 |
| 206145_at | AF178841 | 270 | 2.10E−02 | 3.03E−02 | 9.28E−03 | 1 | 1.995 | RHAG |
| 206609_at | NM_005462 | 271 | 2.10E−02 | 7.04E−03 | 7.65E−04 | 1 | 2.006 | MAGEC1 |
| 215116_s_at | AF035321 | 272 | 2.10E−02 | 7.04E−03 | 3.89E−02 | 1 | 2.021 | DNM1 |
| 208229_at | NM_022975 | 273 | 2.10E−02 | 7.04E−03 | 1.97E−03 | 1 | 2.048 | FGFR2 |
| 211349_at | AB001328 | 274 | 2.10E−02 | 2.60E−02 | 6.74E−04 | 1 | 2.049 | SLC15A1 |
| 207654_x_at | NM_001938 | 275 | 3.70E−03 | 1.36E−03 | 3.30E−03 | 1 | 2.070 | DR1 |
| 209188_x_at | AW516932 | 276 | 3.70E−03 | 1.36E−03 | 3.30E−03 | 1 | 2.070 | DR1 |
| 216652_s_at | AL137673 | 277 | 3.70E−03 | 1.36E−03 | 3.30E−03 | 1 | 2.070 | DR1 |
| 214565_s_at | NM_012390 | 278 | 2.10E−02 | 2.17E−02 | 2.50E−03 | 1 | 2.092 | PROL3, PROL5 |
| 214566_at | NM_012390 | 279 | 2.10E−02 | 2.17E−02 | 2.50E−03 | 1 | 2.092 | PROL5 |
| 215719_x_at | X83493 | 280 | 3.70E−03 | 1.36E−03 | 4.50E−04 | 1 | 2.136 | TNFRSF6 |
| 216252_x_at | Z70519 | 281 | 2.10E−02 | 7.04E−03 | 2.33E−03 | 1 | 2.136 | TNFRSF6 |
| 208405_s_at | NM_006016 | 282 | 3.70E−03 | 3.70E−03 | 1.33E−03 | 1 | 2.137 | CD164 |
| 216857_at | L48728 | 283 | 2.10E−02 | 1.14E−02 | 1.47E−03 | 1 | 2.147 | TCRB PS7 |
| 216865_at | M64108 | 284 | 3.70E−03 | 3.70E−03 | 6.10E−03 | 1 | 2.151 | COL14A1 |
| 216866_s_at | M64108 | 285 | 3.70E−03 | 3.70E−03 | 6.10E−03 | 1 | 2.151 | COL14A1 |
| 212942_s_at | AB033025 | 286 | 3.70E−03 | 1.36E−03 | 8.72E−04 | 1 | 2.192 | KIAA1199 |
| 209737_at | AB014605 | 287 | 3.70E−03 | 1.36E−03 | 6.90E−04 | 1 | 2.199 | AIP1 |
| 207325_x_at | NM_004988 | 288 | 2.10E−02 | 7.04E−03 | 2.44E−02 | 1 | 2.232 | MAGEA1 |
| 215017_s_at | AW270932 | 289 | 3.70E−03 | 3.70E−03 | 3.32E−04 | 1 | 2.233 | C1orf39 |
| 203806_s_at | NM_000135 | 290 | 2.10E−02 | 7.04E−03 | 2.51E−03 | 1 | 2.249 | FANCA |
| 203440_at | M34064 | 291 | 2.10E−02 | 2.60E−02 | 1.61E−02 | 1 | 2.355 | CDH2 |
| 205339_at | NM_003035 | 292 | 2.10E−02 | 7.04E−03 | 8.46E−04 | 1 | 2.355 | SIL |
| 209648_x_at | AL136896 | 293 | 3.70E−03 | 1.36E−03 | 3.23E−03 | 1 | 2.357 | SOCS5 |
| 205429_s_at | NM_016447 | 294 | 2.10E−02 | 7.04E−03 | 1.09E−03 | 1 | 2.361 | MPP6 |
| 208340_at | NM_003723 | 295 | 3.70E−03 | 1.36E−03 | 1.07E−03 | 1 | 2.369 | CASP13 |
| 211203_s_at | U07820 | 296 | 2.10E−02 | 2.60E−02 | 6.09E−03 | 1 | 2.386 | CNTN1 |
| 206437_at | NM_003775 | 297 | 3.70E−03 | 1.36E−03 | 3.29E−03 | 1 | 2.397 | EDG6 |
| 207663_x_at | NM_001473 | 298 | 2.10E−02 | 1.14E−02 | 1.79E−03 | 1 | 2.404 | GAGE3 |
| 207739_s_at | NM_001472 | 299 | 2.10E−02 | 1.14E−02 | 1.79E−03 | 1 | 2.404 | GAGE8, |
| | | | | | | | | GAGE4, |
| | | | | | | | | GAGE5, |
| | | | | | | | | GAGE7, |
| | | | | | | | | GAGE2, |
| | | | | | | | | GAGE1, |
| | | | | | | | | GAGE6, |
| | | | | | | | | GAGE3, |
| | | | | | | | | GAGE7B |
| 203889_at | NM_003020 | 300 | 2.10E−02 | 1.14E−02 | 2.16E−03 | 1 | 2.410 | SGNE1 |
| 217210_at | AL031737 | 301 | 2.10E−02 | 1.14E−02 | 2.41E−03 | 1 | 2.431 | — |
| 204844_at | L12468 | 302 | 3.70E−03 | 1.36E−03 | 2.31E−03 | 1 | 2.461 | ENPEP |
| 214726_x_at | AL556041 | 303 | 2.10E−02 | 1.14E−02 | 1.09E−02 | 1 | 2.466 | ADD1 |
| 206702_at | NM_000459 | 304 | 2.10E−02 | 1.14E−02 | 1.23E−03 | 1 | 2.467 | TEK |
| 209835_x_at | BC004372 | 305 | 2.10E−02 | 3.03E−02 | 4.58E−02 | 1 | 2.468 | CD44 |
| 210916_s_at | AF098641 | 306 | 2.10E−02 | 3.03E−02 | 4.58E−02 | 1 | 2.468 | CD44 |
| 212014_x_at | AI493245 | 307 | 2.10E−02 | 3.03E−02 | 4.58E−02 | 1 | 2.468 | CD44 |
| 203594_at | NM_003729 | 308 | 2.10E−02 | 2.60E−02 | 3.38E−02 | 1 | 2.474 | RTCD1 |
| 206537_at | NM_001167 | 309 | 4.11E−04 | 4.11E−04 | 2.90E−04 | 1 | 2.483 | BIRC4 |
| 201196_s_at | M21154 | 310 | 2.10E−02 | 2.60E−02 | 7.72E−03 | 1 | 2.525 | AMD1 |
| 207705_s_at | NM_025176 | 311 | 2.10E−02 | 3.03E−02 | 3.93E−02 | 1 | 2.532 | KIAA0980 |
| 210992_x_at | U90939 | 312 | 2.10E−02 | 3.03E−02 | 4.34E−02 | 1 | 2.544 | FCGR2C |
| 211395_x_at | U90940 | 313 | 2.10E−02 | 3.03E−02 | 4.34E−02 | 1 | 2.544 | FCGR2C |
| 205446_s_at | NM_001880 | 314 | 2.10E−02 | 1.14E−02 | 4.31E−02 | 1 | 2.552 | ATF2 |
| 204979_s_at | NM_007341 | 315 | 2.10E−02 | 7.04E−03 | 2.37E−03 | 1 | 2.587 | SH3BGR |
| 208525_s_at | NM_012369 | 316 | 2.10E−02 | 1.14E−02 | 2.40E−03 | 1 | 2.639 | OR2F1, OR2F2 |
| 208526_at | NM_012369 | 317 | 2.10E−02 | 1.14E−02 | 2.40E−03 | 1 | 2.639 | OR2F1 |
| 204072_s_at | NM_023037 | 318 | 2.10E−02 | 1.14E−02 | 2.67E−02 | 1 | 2.651 | 13CDNA73 |
| 214151_s_at | AU144243 | 319 | 2.10E−02 | 2.60E−02 | 1.49E−02 | 1 | 2.682 | PIGB |
| 221511_x_at | AF212228 | 320 | 2.10E−02 | 2.60E−02 | 1.49E−02 | 1 | 2.682 | CCPG1 |
| 222156_x_at | AK022459 | 321 | 2.10E−02 | 2.60E−02 | 1.49E−02 | 1 | 2.682 | CCPG1 |
| 207360_s_at | NM_002531 | 322 | 2.10E−02 | 7.04E−03 | 2.96E−03 | 1 | 2.733 | NTSR1 |
| 206577_at | NM_003381 | 323 | 2.10E−02 | 3.03E−02 | 4.09E−02 | 1 | 2.750 | VIP |
| 201756_at | NM_002946 | 324 | 2.10E−02 | 2.60E−02 | 8.61E−03 | 1 | 2.779 | RPA2 |
| 220266_s_at | AF105036 | 325 | 2.10E−02 | 1.14E−02 | 1.09E−02 | 1 | 2.807 | KLF4 |
| 211024_s_at | BC006221 | 326 | 3.70E−03 | 1.36E−03 | 5.86E−03 | 1 | 2.869 | TITF1 |
| 206131_at | NM_001832 | 327 | 2.10E−02 | 1.14E−02 | 2.88E−03 | 1 | 2.879 | CLPS |
| 214642_x_at | AI200443 | 328 | 2.10E−02 | 7.04E−03 | 2.99E−03 | 1 | 2.940 | MAGEA5 |
| 200769_s_at | BC001686 | 329 | 4.11E−04 | 4.11E−04 | 6.60E−04 | 1 | 2.942 | MAT2A |
| 213363_at | AW170549 | 330 | 2.10E−02 | 7.04E−03 | 9.28E−04 | 1 | 2.945 | CASBL |
| 206762_at | NM_002234 | 331 | 2.10E−02 | 2.60E−02 | 1.88E−03 | 1 | 2.952 | KCNA5 |
| 203952_at | NM_007348 | 332 | 2.10E−02 | 7.04E−03 | 4.68E−03 | 1 | 2.967 | ATF6 |
| 201521_s_at | NM_007362 | 333 | 2.10E−02 | 1.14E−02 | 2.09E−03 | 1 | 3.026 | NCBP2 |
| 204227_s_at | NM_004614 | 334 | 2.10E−02 | 2.17E−02 | 4.48E−03 | 1 | 3.055 | TK2 |
| 204643_s_at | NM_006375 | 335 | 4.11E−04 | 4.11E−04 | 2.98E−05 | 1 | 3.068 | COVA1 |
| 204668_at | AL031670 | 336 | 2.10E−02 | 2.60E−02 | 1.94E−02 | 1 | 3.073 | RNF24 |
| 202404_s_at | NM_000089 | 337 | 2.10E−02 | 2.60E−02 | 9.81E−03 | 1 | 3.074 | COL1A2 |
| 210040_at | AF208159 | 338 | 3.70E−03 | 1.36E−03 | 6.35E−03 | 1 | 3.131 | SLC12A5 |
| 215634_at | AF007137 | 339 | 3.70E−03 | 3.70E−03 | 4.86E−04 | 1 | 3.167 | GRIA1 |
| 206091_at | NM_002381 | 340 | 2.10E−02 | 3.03E−02 | 2.69E−03 | 1 | 3.209 | MATN3 |
| 210166_at | AF051151 | 341 | 2.10E−02 | 1.14E−02 | 1.52E−02 | 1 | 3.226 | TLR5 |
| 200713_s_at | NM_012325 | 342 | 2.10E−02 | 3.03E−02 | 2.12E−02 | 1 | 3.241 | MAPRE1 |
| 205732_s_at | NM_006540 | 343 | 3.70E−03 | 3.70E−03 | 6.93E−04 | 1 | 3.257 | NCOA2 |
| 204493_at | NM_001196 | 344 | 2.10E−02 | 2.17E−02 | 8.04E−03 | 1 | 3.263 | BID |
| 207780_at | NM_001340 | 345 | 2.17E−05 | 2.17E−05 | 1.82E−06 | 1 | 3.302 | CYLC2 |
| 217056_at | X61070 | 346 | 2.10E−02 | 3.03E−02 | 1.17E−02 | 1 | 3.434 | TRA@ |
| 209189_at | BC004490 | 347 | 3.70E−03 | 3.70E−03 | 6.05E−03 | 1 | 3.463 | FOS |
| 216392_s_at | AK021846 | 348 | 3.70E−03 | 2.49E−03 | 1.69E−04 | 1 | 3.480 | SEC23IP |
| 215486_at | AW072461 | 349 | 3.70E−03 | 2.49E−03 | 8.93E−03 | 1 | 3.516 | PRPS1L1 |
| 213131_at | R38389 | 350 | 2.10E−02 | 2.17E−02 | 3.62E−03 | 1 | 3.537 | OLFM1 |
| 207681_at | NM_001504 | 351 | 2.10E−02 | 2.17E−02 | 2.78E−02 | 1 | 3.601 | CXCR3 |
| 207224_s_at | NM_016543 | 352 | 2.10E−02 | 7.04E−03 | 3.05E−03 | 1 | 3.879 | SIGLEC7 |
| 207307_at | NM_000868 | 353 | 3.70E−03 | 2.49E−03 | 1.40E−04 | 1 | 4.032 | HTR2C |
| 203676_at | NM_002076 | 354 | 3.70E−03 | 2.49E−03 | 6.66E−04 | 1 | 4.040 | GNS |
| 210729_at | U36269 | 355 | 2.10E−02 | 3.03E−02 | 2.59E−02 | 1 | 4.143 | NPY2R |
| 208743_s_at | BC001359 | 356 | 2.10E−02 | 1.14E−02 | 4.88E−02 | 1 | 4.276 | YWHAB |
| 207643_s_at | NM_001065 | 357 | 2.10E−02 | 1.14E−02 | 5.70E−04 | 1 | 4.356 | TNFRSF1A |
| 205321_at | NM_001415 | 358 | 3.70E−03 | 1.36E−03 | 1.74E−03 | 1 | 4.406 | EIF2S3 |
| 214348_at | NM_001057 | 359 | 3.70E−03 | 1.36E−03 | 1.09E−03 | 1 | 4.453 | TACR2 |
| 206556_at | NM_014410 | 360 | 3.70E−03 | 2.49E−03 | 2.77E−04 | 1 | 4.518 | CLUL1 |
| 216621_at | AL050032 | 361 | 3.70E−03 | 3.70E−03 | 1.12E−03 | 1 | 4.628 | AL050032.1 |
| 203779_s_at | NM_005797 | 362 | 3.70E−03 | 2.49E−03 | 2.04E−03 | 1 | 4.695 | EVA1 |
| 201894_s_at | NM_001920 | 363 | 3.70E−03 | 3.70E−03 | 5.62E−04 | 1 | 4.765 | SSR1 |
| 206826_at | NM_002677 | 364 | 2.10E−02 | 3.03E−02 | 4.05E−02 | 1 | 4.767 | PMP2 |
| 202319_at | NM_015571 | 365 | 4.11E−04 | 2.17E−04 | 1.38E−05 | 1 | 4.842 | SENP6 |
| 210417_s_at | U81802 | 366 | 2.10E−02 | 1.14E−02 | 2.68E−02 | 1 | 4.869 | PIK4CB |
| 208607_s_at | NM_030754 | 367 | 3.70E−03 | 1.36E−03 | 8.25E−04 | 1 | 5.042 | SAA1, SAA2 |
| 214456_x_at | M23699 | 368 | 3.70E−03 | 1.36E−03 | 8.25E−04 | 1 | 5.042 | SAA1 |
| 205126_at | NM_006296 | 369 | 2.10E−02 | 1.14E−02 | 1.06E−02 | 1 | 5.075 | VRK2 |
| 206902_s_at | NM_005728 | 370 | 3.70E−03 | 3.70E−03 | 1.30E−03 | 1 | 5.146 | ENDOGL1 |
| 206903_at | NM_005728 | 371 | 3.70E−03 | 3.70E−03 | 1.30E−03 | 1 | 5.146 | ENDOGL2 |
| 210224_at | AF031469 | 372 | 3.70E−03 | 3.70E−03 | 4.32E−03 | 1 | 5.155 | MR1 |
| 205408_at | NM_004641 | 373 | 2.10E−02 | 7.04E−03 | 5.13E−04 | 1 | 5.249 | MLLT10 |
| 215157_x_at | AI734929 | 374 | 3.70E−03 | 1.36E−03 | 2.13E−03 | 1 | 5.277 | PABPC1 |
| 210996_s_at | U43430 | 375 | 4.11E−04 | 2.17E−04 | 7.55E−04 | 1 | 5.403 | YWHAE |
| 207236_at | NM_003419 | 376 | 2.10E−02 | 7.04E−03 | 3.06E−03 | 1 | 5.517 | ZNF345 |
| 212850_s_at | AA584297 | 377 | 2.10E−02 | 2.60E−02 | 8.55E−03 | 1 | 5.626 | LRP4 |
| 209581_at | BC001387 | 378 | 4.11E−04 | 2.17E−04 | 7.63E−04 | 1 | 5.674 | HRASLS3 |
| 203066_at | NM_014863 | 379 | 2.10E−02 | 1.14E−02 | 4.28E−03 | 1 | 5.675 | GALNAC4S- |
| | | | | | | | | 6ST |
| 200641_s_at | BC003623 | 380 | 2.10E−02 | 3.03E−02 | 4.18E−03 | 1 | 5.731 | YWHAZ |
| 205538_at | NM_003389 | 381 | 2.10E−02 | 2.60E−02 | 4.30E−02 | 1 | 5.793 | CORO2A |
| 204886_at | AL043646 | 382 | 2.10E−02 | 1.14E−02 | 1.17E−03 | 1 | 5.855 | PLK4 |
| 206439_at | NM_004950 | 383 | 2.10E−02 | 2.17E−02 | 4.94E−03 | 1 | 6.348 | DSPG3 |
| 212617_at | AB002293 | 384 | 2.10E−02 | 2.60E−02 | 2.60E−03 | 1 | 6.788 | ZNF609 |
| 212461_at | BF793951 | 385 | 2.10E−02 | 3.03E−02 | 3.54E−02 | 1 | 6.845 | — |
| 214602_at | D17391 | 386 | 2.10E−02 | 1.14E−02 | 3.49E−03 | 1 | 7.218 | COL4A4 |
| 206812_at | NM_000025 | 387 | 3.70E−03 | 2.49E−03 | 6.95E−03 | 1 | 7.330 | ADRB3 |
| 202620_s_at | NM_000935 | 388 | 3.70E−03 | 3.70E−03 | 8.23E−03 | 1 | 7.350 | PLOD2 |
| 206925_at | NM_005668 | 389 | 2.10E−02 | 2.60E−02 | 7.44E−03 | 1 | 7.681 | SIAT8D |
| 205530_at | NM_004453 | 390 | 2.10E−02 | 3.03E−02 | 3.39E−02 | 1 | 7.717 | ETFDH |
| 203834_s_at | NM_006464 | 391 | 3.70E−03 | 1.36E−03 | 7.54E−04 | 1 | 7.727 | TGOLN2 |
| 202923_s_at | NM_001498 | 392 | 2.10E−02 | 7.04E−03 | 1.04E−03 | 1 | 7.744 | GCLC |
| 203517_at | NM_006554 | 393 | 2.10E−02 | 3.03E−02 | 3.91E−03 | 1 | 8.001 | MTX2 |
| 209754_s_at | AF113682 | 394 | 3.70E−03 | 3.70E−03 | 1.91E−03 | 1 | 8.351 | TMPO |
| 207245_at | NM_001077 | 395 | 3.70E−03 | 2.49E−03 | 4.63E−03 | 1 | 8.575 | UGT2B17 |
| 207392_x_at | NM_001076 | 396 | 3.70E−03 | 2.49E−03 | 4.63E−03 | 1 | 8.575 | UGT2B15 |
| 206692_at | NM_002241 | 397 | 2.10E−02 | 7.04E−03 | 3.37E−03 | 1 | 8.819 | KCNJ10 |
| 208050_s_at | NM_001224 | 398 | 4.11E−04 | 2.17E−04 | 2.66E−05 | 1 | 9.015 | CASP2 |
| 210055_at | BE045816 | 399 | 2.10E−02 | 2.60E−02 | 3.07E−03 | 1 | 9.077 | TSHR |
| 205174_s_at | NM_012413 | 400 | 2.10E−02 | 7.04E−03 | 3.72E−03 | 1 | 10.221 | QPCT |
| 210375_at | X83858 | 401 | 3.70E−03 | 2.49E−03 | 8.20E−03 | 1 | 10.333 | PTGER3 |
| 206099_at | NM_006255 | 402 | 2.10E−02 | 1.14E−02 | 1.38E−02 | 1 | 10.583 | PRKCH |
| 203550_s_at | NM_006589 | 403 | 2.10E−02 | 1.14E−02 | 7.88E−04 | 1 | 10.893 | C1orf2 |
| 210331_at | AB048365 | 404 | 2.10E−02 | 3.03E−02 | 1.00E−02 | 1 | 10.931 | NEDL1 |
| 205206_at | NM_000216 | 405 | 3.70E−03 | 1.36E−03 | 1.76E−04 | 1 | 10.990 | KAL1 |
| 215993_at | AF070543 | 406 | 2.10E−02 | 1.14E−02 | 8.81E−03 | 1 | 12.226 | ODZ2 |
| 213090_s_at | AI744029 | 407 | 2.10E−02 | 2.60E−02 | 1.93E−02 | 1 | 12.921 | TAF4 |
| 202430_s_at | NM_021105 | 408 | 2.10E−02 | 2.60E−02 | 6.43E−03 | 1 | 13.006 | PLSCR1 |
| 215996_at | AI446234 | 409 | 3.70E−03 | 2.49E−03 | 2.92E−04 | 1 | 13.631 | pre-TNK |
| 204073_s_at | NM_013279 | 410 | 2.10E−02 | 3.03E−02 | 3.88E−02 | 1 | 14.189 | C11orf9 |
| 215599_at | X83300 | 411 | 2.10E−02 | 3.03E−02 | 1.34E−02 | 1 | 15.668 | SMA4 |
| 207725_at | NM_004575 | 412 | 2.10E−02 | 1.14E−02 | 1.37E−02 | 1 | 16.065 | POU4F2 |
| 212720_at | AI670847 | 413 | 3.70E−03 | 2.49E−03 | 5.17E−04 | 1 | 16.903 | PAPOLA |
| 209459_s_at | AF237813 | 414 | 2.10E−02 | 2.60E−02 | 3.44E−03 | 1 | 17.443 | ABAT |
| 209460_at | AF237813 | 415 | 2.10E−02 | 2.60E−02 | 3.44E−03 | 1 | 17.443 | ABAT |
| 203626_s_at | NM_005983 | 416 | 2.10E−02 | 1.14E−02 | 1.27E−03 | 1 | 17.743 | SKP2 |
| 210567_s_at | BC001441 | 417 | 2.10E−02 | 1.14E−02 | 1.27E−03 | 1 | 17.743 | SKP2 |
| 216984_x_at | D84143 | 418 | 3.70E−03 | 1.36E−03 | 6.07E−03 | 1 | 19.947 | IGLVJ |
| 201817_at | NM_014671 | 419 | 2.10E−02 | 2.17E−02 | 1.70E−03 | 1 | 21.762 | UBE3C |
| 213371_at | AI803302 | 420 | 2.10E−02 | 2.17E−02 | 1.20E−03 | 1 | 28.386 | LDB3 |
| 216887_s_at | AJ133768 | 421 | 2.10E−02 | 2.17E−02 | 1.20E−03 | 1 | 28.386 | LDB3 |
| 204003_s_at | NM_007342 | 422 | 2.10E−02 | 2.17E−02 | 7.68E−04 | 1 | 34.471 | NUPL2 |
| 216430_x_at | AF043586 | 423 | 2.10E−02 | 7.04E−03 | 1.18E−03 | 1 | 37.955 | IGLJ3 |
| 216908_x_at | AF001549 | 424 | 4.11E−04 | 4.11E−04 | 5.94E−04 | 1 | 39.235 | LOC94431 |
| 215719_x_at | X83493 | 425 | 2.10E−02 | 7.04E−03 | 2.33E−03 | 1 | 42.507 | TNFRSF6 |
| 203279_at | NM_014674 | 426 | 2.10E−02 | 2.60E−02 | 2.19E−03 | 1 | 48.486 | EDEM1 |
| 201798_s_at | NM_013451 | 427 | 3.70E−03 | 2.49E−03 | 2.54E−03 | 1 | 131.626 | FER1L3 |
| 208363_s_at | NM_001566 | 428 | 2.10E−02 | 1.14E−02 | 1.99E−02 | 1 | 208.057 | INPP4A |
| 208364_at | NM_001566 | 429 | 2.10E−02 | 1.14E−02 | 1.99E−02 | 1 | 208.057 | INPP4A |
| 206225_at | NM_014910 | 430 | 3.70E−03 | 2.49E−03 | 1.55E−03 | 1 | 326.316 | ZNF507 |
| 211749_s_at | BC005941 | 431 | 2.10E−02 | 2.17E−02 | 1.41E−03 | 1 | NA | VAMP3 |

Altogether, these results demonstrate the MS clinical outcome prediction ability of the identified 431 genes which are differentially expressed between RRMS patients with good or poor clinical outcome.

### Example 2

### Identification of RRMS Clinical Outcome Predicting Genes

Experimental and Statistical Results

Predictive Clinical Outcome Gene Expression Signature—

As is shown in FIG. 6, application of the SVM on data from 19/26 patients with good (9 patients) or poor (10 patients) outcome as a training set, and 9/27 additional patients from the validation group as test set, resulted in a high classification rate of 89%. This high classification was achieved by the Forward feature selection algorithm using 34 gene transcripts (29 genes) (Table 3, hereinbelow) accordingly defined as predictive. Classification rate was 70.4% using only one gene (RRN3) and reached a rate of 85.2% using 6 genes (RRN3, KLF4, HAB1, TPSB2, IGLJ3, COL11A2). Addition of one or all of the remaining predictive genes resulted in maximal classification rate of 89.0%. This suggests that a predictive ability with an accuracy of 89% could be achieved using only 7 genes.

TABLE 3
Genes capable of predicting the clinical outcome of RRMS

| | Corresponding | SEQ | | | | | F/C | |
|---|---|---|---|---|---|---|---|---|
| Probeset | GenBank Acc. | ID | TNOM | Info | t-Test | | (Poor/ | Gene |
| ID | No. | NO: | PValue | PValue | PValue | Dir | Good) | Symbol |
| 203683_s_at | NM_003377 | 156 | 2.10E−02 | 3.03E−02 | 4.63E−03 | −1 | 7.31 | VEGFB |
| 206148_at | NM_002183 | 143 | 2.10E−02 | 3.03E−02 | 2.62E−02 | −1 | 5.17 | IL3RA |
| 207134_x_at | NM_024164 | 127 | 2.10E−02 | 2.17E−02 | 8.61E−03 | −1 | 4.29 | TPSB2 |
| 207532_at | NM_006891 | 46 | 3.70E−03 | 3.70E−03 | 2.66E−02 | −1 | 2.06 | CRYGD |
| 207705_s_at | NM_025176 | 311 | 2.10E−02 | 3.03E−02 | 3.93E−02 | 1 | 2.53 | KIAA0980 |
| 207900_at | NM_002987 | 140 | 2.10E−02 | 1.14E−02 | 6.92E−03 | −1 | 5.13 | CCL17 |
| 208687_x_at | AF352832 | 74 | 3.70E−03 | 3.70E−03 | 3.18E−02 | −1 | 2.47 | HSPA8 |
| 209188_x_at | AW516932 | 276 | 3.70E−03 | 1.36E−03 | 3.30E−03 | 1 | 2.07 | DR1 |
| 209466_x_at | M57399 | 180 | 2.10E−02 | 7.04E−03 | 6.13E−03 | −1 | 21.34 | PTN |
| 209686_at | BC001766 | 182 | 2.10E−02 | 3.03E−02 | 8.52E−03 | −1 | 22.43 | S100B |
| 209726_at | AB018195 | 191 | 3.70E−03 | 1.36E−03 | 1.03E−02 | −1 | 74.85 | CA11 |
| 210328_at | AF101477 | 61 | 2.10E−02 | 2.60E−02 | 3.52E−02 | −1 | 2.26 | GNMT |
| 210916_s_at | AF098641 | 306 | 2.10E−02 | 3.03E−02 | 4.58E−02 | 1 | 2.47 | CD44 |
| 213815_x_at | AI913329 | 115 | 2.10E−02 | 2.60E−02 | 3.04E−03 | −1 | 3.82 | NY-REN |
| | | | | | | | | 24 |
| 214613_at | AW024085 | 97 | 2.10E−02 | 7.04E−03 | 1.72E−02 | −1 | 3.02 | GPR3 |
| 214726_x_at | AL556041 | 303 | 2.10E−02 | 1.14E−02 | 1.09E−02 | 1 | 2.47 | ADD1 |
| 215116_s_at | AF035321 | 272 | 2.10E−02 | 7.04E−03 | 3.89E−02 | 1 | 2.02 | DNM1 |
| 215766_at | AL096729 | 50 | 2.10E−02 | 7.04E−03 | 2.58E−03 | −1 | 2.12 | GSTA1 |
| 215778_x_at | AJ006206 | 16 | 2.10E−02 | 2.60E−02 | 2.29E−02 | −1 | 1.59 | HAB1 |
| 215781_s_at | D87012 | 63 | 2.10E−02 | 2.60E−02 | 2.98E−02 | −1 | 2.27 | TOP3B |
| 215954_s_at | AI200896 | 117 | 2.10E−02 | 2.60E−02 | 3.04E−03 | −1 | 3.82 | NY-REN |
| | | | | | | | | 24 |
| 215993_at | AF070543 | 406 | 2.10E−02 | 1.14E−02 | 8.81E−03 | 1 | 12.23 | ODZ2 |
| 216430_x_at | AF043586 | 423 | 2.10E−02 | 7.04E−03 | 1.18E−03 | 1 | 37.95 | IGLJ3 |
| 216474_x_at | AF206667 | 128 | 2.10E−02 | 2.17E−02 | 8.61E−03 | −1 | 4.29 | TPSAB1, |
| | | | | | | | | TPSB2 |
| 216652_s_at | AL137673 | 277 | 3.70E−03 | 1.36E−03 | 3.30E−03 | 1 | 2.07 | DR1 |
| 216699_s_at | L10038 | 47 | 2.10E−02 | 7.04E−03 | 5.23E−03 | −1 | 2.07 | KLK1 |
| 216875_x_at | X83412 | 17 | 2.10E−02 | 2.60E−02 | 2.29E−02 | −1 | 1.59 | HAB1 |
| 216908_x_at | AF001549 | 424 | 4.11E−04 | 4.11E−04 | 5.94E−04 | 1 | 39.23 | RRN3 |
| 216984_x_at | D84143 | 418 | 3.70E−03 | 1.36E−03 | 6.07E−03 | 1 | 19.95 | IGLVJ |
| 216993_s_at | U32169 | 190 | 2.10E−02 | 2.60E−02 | 1.55E−02 | −1 | 65.43 | COL11A2 |
| 217060_at | U03115 | 139 | 2.10E−02 | 3.03E−02 | 4.35E−02 | −1 | 5.11 | TCRBV |
| 217109_at | AJ242547 | 102 | 3.70E−03 | 3.70E−03 | 2.34E−02 | −1 | 3.21 | MUC4 |
| 217110_s_at | AJ242547 | 103 | 3.70E−03 | 3.70E−03 | 2.34E−02 | −1 | 3.21 | MUC4 |
| 220266_s_at | AF105036 | 325 | 2.10E−02 | 1.14E−02 | 1.09E−02 | 1 | 2.81 | KLF4 |

Independent Validation of the Predictive Clinical Outcome Gene Expression Signature—

Applying the resulting SVM generated classifier, based on the 34 predictive genes to an additional data set of 18/27 patients from the validation group maintained the high classification rate of 88.9%, p<0.00001.

Altogether, these results demonstrate the identification of 34 genes which are capable of predicting the outcome of RRMS (e.g., poor or good clinical outcome) with a classification rate of about 90%.

In addition, these results demonstrate that gene expression profiling combined with carefully chosen learning algorithms allow the prediction of disease outcome and can be incorporated into clinical decision making in relapsing-remitting MS. Since MS has a winding course and the rate of disease progression differs between patients, the results obtained from the present study can predict patient outcome and may be incorporated in individualized tailored management of RRMS. Application of the invention may enable planning of tailored therapeutic strategies and allow delineation of patients at high-risk that may benefit from early therapy.

### Example 3

Biological Regulation of the Predictive Clinical Outcome Gene Expression

Functional Annotation Results

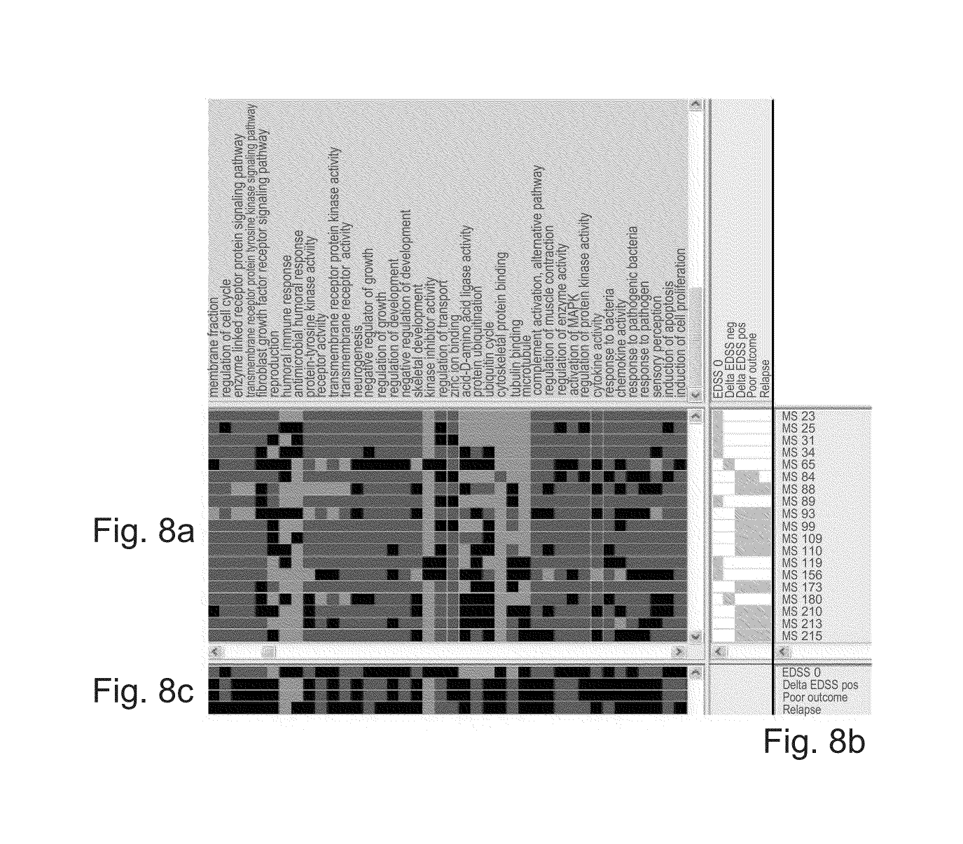

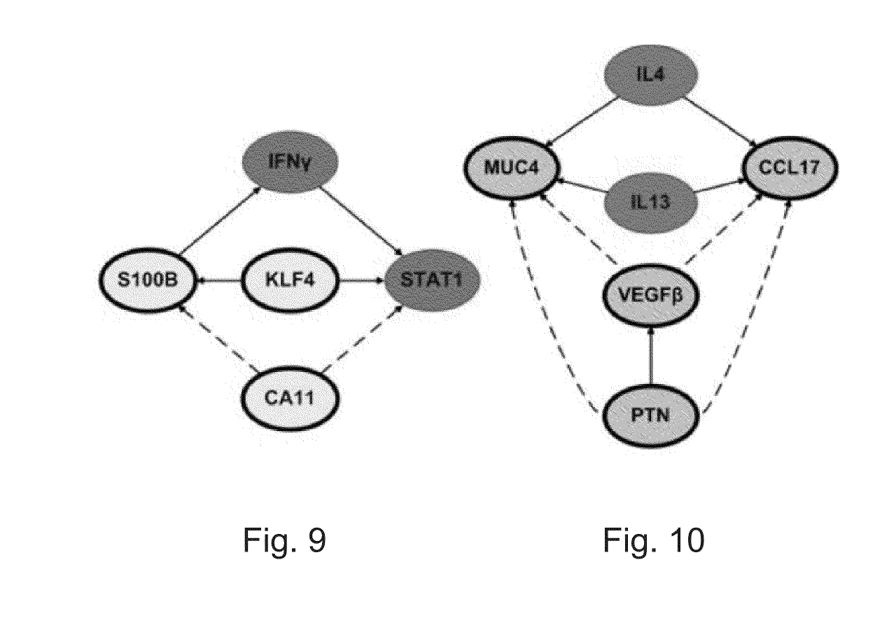

Functional annotation of the 34 predictive genes described in Table 3, Example 2, hereinabove, demonstrated that this group of genes was significantly enriched with zinc-ion binding protein genes (S100B, KLF4, CA11) and with genes exhibiting cytokine activity (CCL17, MUC4, PTN VEGFB), p=0.02 and p=0.005, respectively (FIG. 7). The Genomica software confirmed the enrichment by zinc-ion binding gene family and by cytokine activity genes using all the 431 differentiating gene expression signature data (FIGS. 8a-8c). Using these enriched gene-families, regulatory pathways were reconstructed (FIGS. 9 and 10). These pathways suggest that apoptosis regulation through zinc-ion binding and cytokine activity is responsible for Th1/Th2 cytokine activity shift and may play a role in the clinical outcome of RRMS. Genomica reconstruction of regulatory gene expression networks based on all 431 differentiating genes resulted in a regulation pathway in which the predictive zinc-ion binding gene KLF4 in association with CLPP and RRLP mediate downstream genes including S100B (FIG. 10). Other interesting functional groups in the 29 predictive genes include adhesion and cell migration like CD44 and COL11A2, and T cell receptor genes like TCRVB, all play an important role in MS pathogenesis.

### Example 4

### Selection of Differentiating Genes Computational Results

Selection of Differentiating Genes and Determination of their Predictive Power—

To evaluate the power of each of the 431 differentiating genes identified in this study to predict the prognosis (good or poor clinical outcome) of a subject diagnosed with multiple sclerosis, the study sample was randomly divided into 80% of the subjects as a “training set” and 20% of the subjects as a “test set” and a model was build using the SVM based on RBF kernel. For each of the differentiating genes the predictability of the training set on the clinical outcome of the test set was computed and the average error following 50 permutations was calculated. Genes with the lowest average error were selected, then, for each selected gene, the remaining genes were added one after the other, by selecting the next gene such that the average error after 50 repeats of the group of genes including the new gene has the lowest average error as compared to the addition of another gene. This process was repeated 430 times for each additional genes added to the previous group of genes. The resulting average error plot is shown in FIG. 12, and the average error for each gene combination is demonstrated in Table 4, hereinbelow, wherein the first gene in row number 1 (SEQ ID NO:158; NM_{—}005012) exhibits the best predictive power (error average of “0”).

TABLE 4
Average error of gene combination with predictive ability of
Multiple Sclerosis clinical outcome

| Row | | | | Average | |
|---|---|---|---|---|---|
| Number | SEQ ID NO: | Probeset ID | Gene Bank ID | error | Gene Symbol |
| 1 | 158 | 205805_s_at | NM_005012 | 0 | ROR1 |
| 2 | 68 | 200949_x_at | NM_001023 | 0 | RPS20 |
| 3 | 5 | 201783_s_at | NM_021975 | 0 | RELA |
| 4 | 58 | 200647_x_at | NM_003752 | 0 | EIF3S8 |
| 5 | 329 | 200769_s_at | NM_005911 | 0 | MAT2A |
| 6 | 120 | 200936_at | NM_000973 | 0 | RPL8 |
| 7 | 380 | 200641_s_at | U28964 | 0 | YWHAZ |
| 8 | 342 | 200713_s_at | NM_012325 | 0 | MAPRE1 |
| 9 | 88 | 200790_at | NM_002539 | 0 | ODC1 |
| 10 | 166 | 200834_s_at | NM_001024 | 0 | RPS21 |
| 11 | 266 | 200889_s_at | AI016620 | 0 | SSR1 |
| 12 | 51 | 201023_at | NM_005642 | 0 | TAF7 |
| 13 | 310 | 201196_s_at | M21154 | 0 | AMD1 |
| 14 | 91 | 201513_at | AI659180 | 0 | TSN |
| 15 | 427 | 201798_s_at | NM_013451 | 0 | FLR1L3 |
| 16 | 22 | 201889_at | NM_014888 | 0 | FAM3C |
| 17 | 84 | 201418_s_at | NM_003107 | 0 | SOX4 |
| 18 | 269 | 200771_at | NM_002293 | 0 | LAMC1 |
| 19 | 388 | 202620_s_at | NM_000935 | 0 | PLOD2 |
| 20 | 155 | 201497_x_at | NM_022844 | 0 | MYH11 |
| 21 | 333 | 201521_s_at | NM_007362 | 0 | NCBP2 |
| 22 | 215 | 201538_s_at | NM_004090 | 0 | DUSP3 |
| 23 | 195 | 202270_at | NM_002053 | 0 | GBP1 |
| 24 | 419 | 201817_at | NM_014671 | 0 | KIAA0010/UBE3C |
| 25 | 75 | 202314_at | NM_000786 | 0 | CYP51A1 |
| 26 | 125 | 204197_s_at | NM_004350 | 0 | RUNX3 |
| 27 | 11 | 203205_at | NM_014663 | 0 | JMJD2 |
| 28 | 251 | 201453_x_at | NM_005614 | 0 | RHEB |
| 29 | 253 | 202805_s_at | NM_004996 | 0 | ABCC1 |
| 30 | 337 | 202404_s_at | NM_000089 | 0 | COL1A2 |
| 31 | 110 | 202140_s_at | NM_003992 | 0 | CLK3 |
| 32 | 222 | 203266_s_at | NM_003010 | 0 | MAP2K4 |
| 33 | 56 | 202799_at | NM_006012 | 0 | CLPP |
| 34 | 324 | 201756_at | NM_002946 | 0 | RPA2 |
| 35 | 156 | 203683_s_at | NM_003377 | 0 | VEGFB |
| 36 | 7 | 203145_at | NM_006461 | 0 | SPAG5 |
| 37 | 57 | 202127_at | AB011108 | 0 | PRPF4B |
| 38 | 233 | 202260_s_at | NM_003165 | 0 | STXBP1 |
| 39 | 149 | 202268_s_at | NM_003905 | 0 | APPBP1 |
| 40 | 363 | 201894_s_at | NM_001920 | 0 | DCN |
| 41 | 107 | 203422_at | NM_002691 | 0 | POLD1 |
| 42 | 193 | 203503_s_at | NM_004565 | 0 | PEX14 |
| 43 | 393 | 203517_at | NM_006554 | 0 | MTX2 |
| 44 | 265 | 202886_s_at | M65254 | 0 | PPP2R1B |
| 45 | 160 | 203329_at | NM_002845 | 0 | PTPRM |
| 46 | 41 | 202732_at | NM_007066 | 0 | PKIG |
| 47 | 38 | 203022_at | NM_006397 | 0 | RNASEH2A |
| 48 | 90 | 203340_s_at | NM_003705 | 0 | SLC25A12 |
| 49 | 70 | 203701_s_at | NM_017722 | 0 | FLJ20244 |
| 50 | 85 | 202410_x_at | NM_000612 | 0 | IGF2 |
| 51 | 403 | 203550_s_at | NM_006589 | 0 | C1orf2 |
| 52 | 304 | 206702_at | NM_000459 | 0 | TEK |
| 53 | 426 | 203279_at | NM_014674 | 0 | EDEM1 |
| 54 | 240 | 205261_at | NM_002630 | 0 | PGC |
| 55 | 49 | 203193_at | NM_004451 | 0 | ESRRA |
| 56 | 294 | 205429_s_at | NM_016447 | 0 | MPP6 |
| 57 | 136 | 202415_s_at | NM_012267 | 0 | HSPBP1 |
| 58 | 150 | 202909_at | NM_014805 | 0 | EPM2AIP1 |
| 59 | 232 | 205990_s_at | NM_003392 | 0 | WNT5A |
| 60 | 10 | 203426_s_at | M65062 | 0 | IGFBP5 |
| 61 | 392 | 202923_s_at | NM_001498 | 0 | GCLC |
| 62 | 89 | 203339_at | AI887457 | 0 | SLC25A12 |
| 63 | 332 | 203952_at | NM_007348 | 0 | ATF6 |
| 64 | 290 | 203806_s_at | NM_000135 | 0 | FANCA |
| 65 | 422 | 204003_s_at | NM_007342 | 0 | NUPL2 |
| 66 | 291 | 203440_at | M34064 | 0 | CDH2 |
| 67 | 114 | 202554_s_at | AL527430 | 0 | GSTM3 |
| 68 | 309 | 206537_at | NM_001167 | 0 | BIRC4 |
| 69 | 203 | 206421_s_at | NM_003784 | 0 | SERPINB7 |
| 70 | 362 | 203779_s_at | NM_005797 | 0 | EVA1 |
| 71 | 397 | 206692_at | NM_002241 | 0 | KCNJ10 |
| 72 | 334 | 204227_s_at | NM_004614 | 0 | TK2 |
| 73 | 302 | 204844_at | L12468 | 0 | ENPEP |
| 74 | 179 | 203851_at | NM_002178 | 0 | IGFBP6 |
| 75 | 171 | 208733_at | AW301641 | 0 | RAB2 |
| 76 | 53 | 204319_s_at | NM_002925 | 0 | RGS10 |
| 77 | 402 | 206099_at | NM_006255 | 0 | PRKCH |
| 78 | 315 | 204979_s_at | NM_007341 | 0 | SH3BGR |
| 79 | 271 | 206609_at | NM_005462 | 0 | MAGEC1 |
| 80 | 218 | 205062_x_at | NM_002892 | 0 | RBBP1 |
| 81 | 154 | 202312_s_at | NM_000088 | 0 | COL1A1 |
| 82 | 243 | 207010_at | NM_000812 | 0 | GABRB1 |
| 83 | 211 | 203501_at | NM_006102 | 0 | PGCP |
| 84 | 180 | 209466_x_at | M57399 | 0 | PTN |
| 85 | 412 | 207725_at | NM_004575 | 0 | POU4F2 |
| 86 | 300 | 203889_at | NM_003020 | 0 | SGNE1 |
| 87 | 131 | 212674_s_at | AK002076 | 0 | DHX30 |
| 88 | 71 | 210463_x_at | BC002492 | 0 | FLJ20244 |
| 89 | 398 | 208050_s_at | NM_001224 | 0 | CASP2 |
| 90 | 289 | 215017_s_at | AW270932 | 0 | FLJ20275 |
| 91 | 371 | 206903_at | NM_005728 | 0 | ENDOGL1 |
| 92 | 118 | 204144_s_at | NM_004204 | 0 | PIGQ |
| 93 | 220 | 212976_at | R41498 | 0 | TA-LRRP |
| 94 | 82 | 203801_at | AA013164 | 0 | SIP |
| 95 | 42 | 205013_s_at | NM_000675 | 0 | ADORA2A |
| 96 | 430 | 206225_at | NM_014910 | 0 | KIAA1084 |
| 97 | 64 | 204163_at | NM_007046 | 0 | EMILIN1 |
| 98 | 144 | 204816_s_at | NM_014681 | 0 | DHX34 |
| 99 | 2 | 205034_at | NM_004702 | 0 | CCNE2 |
| 100 | 205 | 204202_at | NM_017604 | 0 | KIAA1023 |
| 101 | 405 | 205206_at | NM_000216 | 0 | KAL1 |
| 102 | 318 | 204072_s_at | NM_023037 | 0 | 13CDNA73 |
| 103 | 146 | 206398_s_at | NM_001770 | 0 | CD19 |
| 104 | 314 | 205446_s_at | NM_001880 | 0 | ATF2 |
| 105 | 12 | 203324_s_at | NM_001233 | 0 | CAV2 |
| 106 | 416 | 203626_s_at | NM_005983 | 0 | SKP2 |
| 107 | 267 | 206942_s_at | NM_002674 | 0 | PMCH |
| 108 | 105 | 206759_at | NM_002002 | 0.005 | FCER2 |
| 109 | 353 | 207307_at | NM_000868 | 0 | HTR2C |
| 110 | 296 | 211203_s_at | U07820 | 0 | CNTN1 |
| 111 | 224 | 207037_at | NM_003839 | 0.005 | TNFRSF11A |
| 112 | 165 | 206516_at | NM_000479 | 0 | AMH |
| 113 | 113 | 208041_at | NM_002929 | 0 | RHOK |
| 114 | 345 | 207780_at | NM_001340 | 0 | CYLC2 |
| 115 | 387 | 206812_at | NM_000025 | 0.005 | ADRB3 |
| 116 | 61 | 210328_at | AF101477 | 0 | GNMT |
| 117 | 250 | 214099_s_at | AK001619 | 0.005 | PDE4DIP |
| 118 | 59 | 210949_s_at | BC000533 | 0.005 | EIF3S8 |
| 119 | 235 | 211561_x_at | L35253 | 0.005 | MAPK14 |
| 120 | 382 | 204886_at | AL043646 | 0.005 | STK18 |
| 121 | 143 | 206148_at | NM_002183 | 0.005 | IL3RA |
| 122 | 361 | 216621_at | AL050032 | 0 | — |
| 123 | 372 | 210224_at | AF031469 | 0.005 | MR1 |
| 124 | 199 | 209413_at | BC002431 | 0 | B4GALT2 |
| 125 | 79 | 206879_s_at | NM_013982 | 0 | NRG2 |
| 126 | 116 | 214892_x_at | BC004262 | 0 | NY-REN-24 |
| 127 | 162 | 208580_x_at | NM_021968 | 0.005 | HIST1H4J |
| 128 | 322 | 207360_s_at | NM_002531 | 0.005 | NTSR1 |
| 129 | 354 | 203676_at | NM_002076 | 0 | GNS |
| 130 | 391 | 203834_s_at | NM_006464 | 0.01 | TGOLN2 |
| 131 | 377 | 212850_s_at | AA584297 | 0.005 | LRP4 |
| 132 | 255 | 205619_s_at | NM_004527 | 0.005 | MEOX1 |
| 133 | 270 | 206145_at | NM_000324 | 0.005 | RHAG |
| 134 | 373 | 205408_at | NM_004641 | 0.01 | MLLT10 |
| 135 | 104 | 212852_s_at | AL538601 | 0.005 | SSA2 |
| 136 | 400 | 205174_s_at | NM_012413 | 0.01 | QPCT |
| 137 | 67 | 222206_s_at | AA781143 | 0.005 | LOC56926 |
| 138 | 167 | 208105_at | NM_000164 | 0.005 | GIPR |
| 139 | 423 | 216430_x_at | AF043586 | 0.005 | IGLJ3 |
| 140 | 188 | 208227_x_at | NM_021721 | 0.01 | ADAM22 |
| 141 | 182 | 209686_at | BC001766 | 0.005 | S100B |
| 142 | 106 | 206760_s_at | NM_002002 | 0.015 | FCER2 |
| 143 | 54 | 209477_at | BC000738 | 0.015 | EMD |
| 144 | 326 | 211024_s_at | BC006221 | 0.005 | TITF1 |
| 145 | 164 | 208091_s_at | NM_030796 | 0.01 | DKFZP564K0822 |
| 146 | 307 | 212014_x_at | AI493245 | 0 | CD44 |
| 147 | 383 | 206439_at | NM_004950 | 0.01 | DSPG3 |
| 148 | 260 | 205962_at | NM_002577 | 0.005 | PAK2 |
| 149 | 340 | 206091_at | NM_002381 | 0.01 | MATN3 |
| 150 | 357 | 207643_s_at | NM_001065 | 0.005 | TNFRSF1A |
| 151 | 390 | 205530_at | NM_004453 | 0.01 | ETFDH |
| 152 | 161 | 207036_x_at | NM_000836 | 0.005 | GRIN2D |
| 153 | 6 | 213457_at | BF739959 | 0.005 | MFHAS1 |
| 154 | 316 | 208525_s_at | NM_012369 | 0.005 | OR2F1 |
| 155 | 272 | 215116_s_at | AF035321 | 0.01 | DNM1 |
| 156 | 338 | 210040_at | AF208159 | 0.01 | SLC12A5 |
| 157 | 241 | 207406_at | NM_000780 | 0 | CYP7A1 |
| 158 | 367 | 208607_s_at | NM_030754 | 0.005 | SAA2 |
| 159 | 379 | 203066_at | NM_014863 | 0.01 | GALNAC4S-6ST |
| 160 | 40 | 212242_at | AL565074 | 0.005 | TUBA1 |
| 161 | 381 | 205538_at | NM_003389 | 0.015 | CORO2A |
| 162 | 231 | 205312_at | NM_003120 | 0.01 | SPI1 |
| 163 | 39 | 203071_at | NM_004636 | 0.015 | SEMA3B |
| 164 | 256 | 215130_s_at | AC002550 | 0.02 | MGC35048 |
| 165 | 286 | 212942_s_at | AB033025 | 0.02 | KIAA1199 |
| 166 | 8 | 210822_at | U72513 | 0.015 | na |
| 167 | 151 | 205798_at | NM_002185 | 0.005 | IL7R |
| 168 | 399 | 210055_at | BE045816 | 0.01 | TSHR |
| 169 | 33 | 205272_s_at | NM_006250 | 0.02 | PRH1 |
| 170 | 254 | 215008_at | AA582404 | 0.01 | TLL2 |
| 171 | 295 | 208340_at | NM_003723 | 0.025 | — |
| 172 | 141 | 205189_s_at | NM_000136 | 0.02 | FANCC |
| 173 | 429 | 208364_at | NM_001566 | 0.02 | INPP4A |
| 174 | 65 | 207954_at | NM_002050 | 0.015 | GATA2 |
| 175 | 229 | 205141_at | NM_001145 | 0.03 | RNASE4 |
| 176 | 259 | 216052_x_at | AF115765 | 0.025 | ARTN |
| 177 | 355 | 210729_at | U32500 | 0.02 | NPY2R |
| 178 | 298 | 207663_x_at | NM_001473 | 0.02 | GAGE4 |
| 179 | 173 | 214481_at | NM_003514 | 0.02 | HIST1H2AM |
| 180 | 371 | 206903_at | NM_005728 | 0.025 | ENDOGL1 |
| 181 | 86 | 204400_at | NM_005864 | 0.015 | EFS |
| 182 | 45 | 208059_at | NM_005201 | 0.02 | CCR8 |
| 183 | 305 | 209835_x_at | BC004372 | 0.03 | CD44 |
| 184 | 127 | 207134_x_at | NM_024164 | 0.025 | TPSB2 |
| 185 | 133 | 210619_s_at | AF173154 | 0.03 | HYAL1 |
| 186 | 200 | 214507_s_at | NM_014285 | 0.025 | RRP4 |
| 187 | 313 | 211395_x_at | U90940 | 0.015 | FCGR2B |
| 188 | 370 | 206902_s_at | NM_005728 | 0.025 | ENDOGL1 |
| 189 | 112 | 210249_s_at | U59302 | 0.025 | NCOA1 |
| 190 | 226 | 208359_s_at | NM_004981 | 0.03 | KCNJ4 |
| 191 | 249 | 209260_at | BC000329 | 0.025 | SFN |
| 192 | 80 | 205463_s_at | NM_002607 | 0.025 | PDGFA |
| 193 | 299 | 207739_s_at | NM_001472 | 0.025 | GAGE1 |
| 194 | 196 | 201975_at | NM_002956 | 0.025 | RSN |
| 195 | 27 | 209031_at | AL519710 | 0.025 | IGSF4 |
| 196 | 308 | 203594_at | NM_003729 | 0.03 | RTCD1 |
| 197 | 288 | 207325_x_at | NM_004988 | 0.025 | MAGEA1 |
| 198 | 349 | 215486_at | AW072461 | 0.03 | LOC221823 |
| 199 | 108 | 202766_s_at | NM_000138 | 0.04 | FBN1 |
| 200 | 311 | 207705_s_at | NM_025176 | 0.035 | KIAA0980 |
| 201 | 14 | 217165_x_at | M10943 | 0.04 | MT1F |
| 202 | 130 | 214488_at | NM_002886 | 0.035 | RAP2B |
| 203 | 285 | 216866_s_at | M64108 | 0.035 | COL14A1 |
| 204 | 207 | 215906_at | S65921 | 0.045 | — |
| 205 | 365 | 202319_at | NM_015571 | 0.035 | SUSP1 |
| 206 | 275 | 207654_x_at | NM_001938 | 0.025 | DR1 |
| 207 | 284 | 216865_at | M64108 | 0.03 | COL14A1 |
| 208 | 96 | 212999_x_at | AW276186 | 0.04 | HLA-DQB1 |
| 209 | 172 | 221847_at | BF665706 | 0.035 | — |
| 210 | 76 | 214095_at | AW190316 | 0.03 | LOC56901 |
| 211 | 279 | 214566_at | NM_012390 | 0.03 | PROL5 |
| 212 | 413 | 212720_at | AI670847 | 0.03 | PAPOLA |
| 213 | 351 | 207681_at | NM_001504 | 0.035 | CXCR3 |
| 214 | 202 | 206364_at | NM_014875 | 0.045 | KIF14 |
| 215 | 37 | 212534_at | AU144066 | 0.035 | ZNF24 |
| 216 | 74 | 208687_x_at | AF352832 | 0.03 | HSPA8 |
| 217 | 375 | 210996_s_at | U43430 | 0.035 | YWHAE |
| 218 | 360 | 206556_at | NM_014410 | 0.035 | CLUL1 |
| 219 | 170 | 202315_s_at | NM_004327 | 0.03 | BCR |
| 220 | 147 | 212540_at | BG476661 | 0.03 | CDC34 |
| 221 | 151 | 205798_at | NM_002185 | 0.04 | IL7R |
| 222 | 306 | 210916_s_at | AF098641 | 0.035 | CD44 |
| 223 | 366 | 210417_s_at | U81802 | 0.05 | PIK4CB |
| 224 | 217 | 205885_s_at | L12002 | 0.045 | ITGA4 |
| 225 | 394 | 209754_s_at | AF113682 | 0.035 | TMPO |
| 226 | 192 | 206944_at | AF007141 | 0.045 | HTR6 |
| 227 | 341 | 210166_at | AF051151 | 0.045 | TLR5 |
| 228 | 352 | 207224_s_at | NM_016543 | 0.04 | SIGLEC7 |
| 229 | 83 | 215746_at | L34409 | 0.03 | — |
| 230 | 157 | 210438_x_at | M25077 | 0.03 | SSA2 |
| 231 | 122 | 206327_s_at | NM_004933 | 0.05 | CDH15 |
| 232 | 350 | 213131_at | R38389 | 0.04 | OLFM1 |
| 233 | 30 | 204066_s_at | NM_014914 | 0.035 | CENTG2 |
| 234 | 25 | 213262_at | AI932370 | 0.04 | SACS |
| 235 | 260 | 205962_at | NM_002577 | 0.045 | PAK2 |
| 236 | 238 | 208724_s_at | BC000905 | 0.04 | RAB1A |
| 237 | 428 | 208363_s_at | NM_001566 | 0.05 | INPP4A |
| 238 | 278 | 214565_s_at | NM_012390 | 0.04 | PROL5 |
| 239 | 252 | 213404_s_at | BF033683 | 0.045 | — |
| 240 | 395 | 207245_at | NM_001077 | 0.055 | UGT2B17 |
| 241 | 343 | 205732_s_at | NM_006540 | 0.05 | NCOA2 |
| 242 | 325 | 220266_s_at | NM_004235 | 0.045 | KLF4 |
| 243 | 31 | 206846_s_at | NM_006044 | 0.05 | HDAC6 |
| 244 | 386 | 214602_at | D17391 | 0.05 | COL4A4 |
| 245 | 301 | 217210_at | AL031737 | 0.045 | — |
| 246 | 317 | 208526_at | NM_012369 | 0.065 | OR2F1 |
| 247 | 29 | 212912_at | AI992251 | 0.05 | RPS6KA2 |
| 248 | 407 | 213090_s_at | AI744029 | 0.05 | TAF4 |
| 249 | 72 | 204703_at | NM_006531 | 0.04 | TG737 |
| 250 | 283 | 216857_at | L48728 | 0.045 | — |
| 251 | 227 | 211451_s_at | U24056 | 0.055 | KCNJ4 |
| 252 | 191 | 209726_at | AB018195 | 0.05 | CA11 |
| 253 | 126 | 205683_x_at | NM_003294 | 0.06 | TPSB2 |
| 254 | 132 | 209253_at | AF037261 | 0.055 | SCAM-1 |
| 255 | 187 | 208226_x_at | NM_004194 | 0.045 | ADAM22 |
| 256 | 94 | 209320_at | AF033861 | 0.05 | ADCY3 |
| 257 | 66 | 210358_x_at | BC002557 | 0.05 | GATA2 |
| 258 | 109 | 216065_at | AL031228 | 0.05 | C6orf11 |
| 259 | 46 | 207532_at | NM_006891 | 0.03 | CRYGD |
| 260 | 212 | 208454_s_at | NM_016134 | 0.06 | PGCP |
| 261 | 24 | 206910_x_at | NM_005666 | 0.055 | HFL3 |
| 262 | 69 | 214003_x_at | BF184532 | 0.055 | RPS20 |
| 263 | 425 | 215719_x_at | X83493 | 0.06 | TNFRSF6 |
| 264 | 1 | 207160_at | NM_000882 | 0.05 | IL12A |
| 265 | 347 | 209189_at | BC004490 | 0.065 | FOS |
| 266 | 197 | 206584_at | NM_015364 | 0.065 | LY96 |
| 267 | 263 | 202166_s_at | NM_006241 | 0.06 | PPP1R2 |
| 268 | 273 | 208229_at | NM_022975 | 0.06 | FGFR2 |
| 269 | 344 | 204493_at | NM_001196 | 0.07 | BID |
| 270 | 181 | 211737_x_at | BC005916 | 0.065 | PTN |
| 271 | 177 | 221551_x_at | AB035172 | 0.065 | SIAT7D |
| 272 | 356 | 208743_s_at | BC001359 | 0.065 | YWHAB |
| 273 | 257 | 215131_at | AC002550 | 0.065 | MGC35048 |
| 274 | 148 | 207694_at | NM_000307 | 0.07 | POU3F4 |
| 275 | 244 | 217301_x_at | X71810 | 0.065 | RBBP4 |
| 276 | 13 | 207509_s_at | NM_002288 | 0.07 | LAIR2 |
| 277 | 73 | 216582_at | AL021808 | 0.07 | — |
| 278 | 420 | 213371_at | AI803302 | 0.06 | LDB3 |
| 279 | 36 | 209156_s_at | AY029208 | 0.065 | COL6A2 |
| 280 | 236 | 216817_s_at | AJ302604 | 0.065 | — |
| 281 | 210 | 207867_at | NM_006193 | 0.08 | PAX4 |
| 282 | 43 | 221792_at | AW118072 | 0.08 | — |
| 283 | 174 | 214644_at | BF061074 | 0.07 | HIST1H2AK |
| 284 | 121 | 204065_at | NM_004854 | 0.055 | CHST10 |
| 285 | 138 | 207938_at | NM_015886 | 0.075 | PI15 |
| 286 | 87 | 210880_s_at | AB001467 | 0.075 | EFS |
| 287 | 194 | 214994_at | BF508948 | 0.065 | KIAA0907 |
| 288 | 389 | 206925_at | NM_005668 | 0.085 | SIAT8D |
| 289 | 44 | 208135_at | NM_006481 | 0.065 | TCF2 |
| 290 | 396 | 207392_x_at | NM_001076 | 0.08 | UGT2B15 |
| 291 | 184 | 210229_s_at | M11734 | 0.085 | CSF2 |
| 292 | 408 | 202430_s_at | NM_021105 | 0.07 | PLSCR1 |
| 293 | 242 | 206218_at | NM_002364 | 0.08 | MAGEB2 |
| 294 | 152 | 203421_at | NM_006034 | 0.08 | TP53I11 |
| 295 | 47 | 216699_s_at | L10038 | 0.065 | KLK1 |
| 296 | 268 | 207862_at | NM_006760 | 0.085 | UPK2 |
| 297 | 128 | 216474_x_at | AF206667 | 0.075 | TPSB2 |
| 298 | 230 | 216695_s_at | AF082559 | 0.085 | TNKS |
| 299 | 225 | 206911_at | NM_005082 | 0.085 | ZNF147 |
| 300 | 431 | 211749_s_at | BC005941 | 0.07 | VAMP3 |
| 301 | 60 | 215230_x_at | AA679705 | 0.085 | EIF3S8 |
| 302 | 206 | 207872_s_at | NM_006863 | 0.085 | LILRB1 |
| 303 | 424 | 216908_x_at | AF001549 | 0.085 | — |
| 304 | 378 | 209581_at | BC001387 | 0.085 | HRASLS3 |
| 305 | 358 | 205321_at | NM_001415 | 0.085 | EIF2S3 |
| 306 | 262 | 203755_at | NM_001211 | 0.09 | BUB1B |
| 307 | 246 | 211109_at | U31601 | 0.075 | JAK3 |
| 308 | 98 | 201269_s_at | AB028991 | 0.1 | KIAA1068 |
| 309 | 320 | 221511_x_at | AB033080 | 0.095 | CPR8 |
| 310 | 117 | 215954_s_at | AI200896 | 0.075 | NY-REN-24 |
| 311 | 401 | 210375_at | X83858 | 0.105 | PTGER3 |
| 312 | 404 | 210331_at | AB048365 | 0.09 | KIAA0322 |
| 313 | 264 | 206570_s_at | NM_002785 | 0.08 | PSG11 |
| 314 | 34 | 214534_at | NM_005322 | 0.1 | HIST1H1B |
| 315 | 303 | 214726_x_at | AL556041 | 0.085 | ADD1 |
| 316 | 369 | 205126_at | NM_006296 | 0.09 | VRK2 |
| 317 | 92 | 204159_at | NM_001262 | 0.095 | CDKN2C |
| 318 | 277 | 216652_s_at | AL137673 | 0.095 | DR1 |
| 319 | 93 | 211792_s_at | U17074 | 0.11 | CDKN2C |
| 320 | 293 | 209648_x_at | AL136896 | 0.11 | SOCS5 |
| 321 | 153 | 207403_at | NM_003604 | 0.105 | IRS4 |
| 322 | 142 | 208009_s_at | NM_014448 | 0.115 | ARHGEF16 |
| 323 | 16 | 215778_x_at | AJ006206 | 0.11 | HAB1 |
| 324 | 359 | 214348_at | NM_001057 | 0.09 | TACR2 |
| 325 | 406 | 215993_at | AF070543 | 0.1 | — |
| 326 | 425 | 215719_x_at | X83493 | 0.115 | TNFRSF6 |
| 327 | 327 | 206131_at | NM_001832 | 0.09 | CLPS |
| 328 | 321 | 222156_x_at | AK022459 | 0.09 | CPR8 |
| 329 | 204 | 209961_s_at | M60718 | 0.11 | HGF |
| 330 | 384 | 212617_at | AB002293 | 0.12 | KIAA0295 |
| 331 | 175 | 217192_s_at | AL022067 | 0.12 | PRDM1 |
| 332 | 331 | 206762_at | NM_002234 | 0.115 | KCNA5 |
| 333 | 297 | 206437_at | NM_003775 | 0.115 | EDG6 |
| 334 | 410 | 204073_s_at | NM_013279 | 0.125 | C11orf9 |
| 335 | 111 | 209107_x_at | U19179 | 0.09 | NCOA1 |
| 336 | 209 | 211832_s_at | AF201370 | 0.145 | MDM2 |
| 337 | 101 | 204895_x_at | NM_004532 | 0.115 | MUC4 |
| 338 | 335 | 204643_s_at | NM_006375 | 0.115 | COVA1 |
| 339 | 213 | 206426_at | NM_005511 | 0.115 | MLANA |
| 340 | 234 | 214624_at | AA548647 | 0.145 | UPK1A |
| 341 | 178 | 215266_at | AL096732 | 0.125 | DNAH3 |
| 342 | 124 | 205608_s_at | U83508 | 0.12 | ANGPT1 |
| 343 | 336 | 204668_at | AL031670 | 0.135 | RNF24 |
| 344 | 414 | 209459_s_at | AF237813 | 0.1 | NPD009 |
| 345 | 19 | 209685_s_at | M13975 | 0.15 | PRKCB1 |
| 346 | 319 | 214151_s_at | AU144243 | 0.14 | CPR8 |
| 347 | 418 | 216984_x_at | D84143 | 0.135 | IGLJ3 |
| 348 | 376 | 207236_at | NM_003419 | 0.145 | ZNF345 |
| 349 | 411 | 215599_at | X83300 | 0.13 | SMA3 |
| 350 | 421 | 216887_s_at | AJ133768 | 0.145 | LDB3 |
| 351 | 348 | 216392_s_at | AK021846 | 0.135 | SEC23IP |
| 352 | 81 | 205022_s_at | NM_005197 | 0.13 | CHES1 |
| 353 | 374 | 215157_x_at | AI734929 | 0.14 | PABPC1 |
| 354 | 140 | 207900_at | NM_002987 | 0.16 | CCL17 |
| 355 | 62 | 211988_at | BG289800 | 0.145 | SMARCE1 |
| 356 | 20 | 207504_at | NM_005182 | 0.13 | CA7 |
| 357 | 168 | 207959_s_at | NM_004662 | 0.155 | DNAH9 |
| 358 | 129 | 214487_s_at | NM_002886 | 0.15 | RAP2B |
| 359 | 415 | 209460_at | AF237813 | 0.13 | NPD009 |
| 360 | 312 | 210992_x_at | U90939 | 0.115 | FCGR2A |
| 361 | 282 | 208405_s_at | NM_006016 | 0.185 | CD164 |
| 362 | 370 | 206902_s_at | NM_005728 | 0.175 | ENDOGL1 |
| 363 | 28 | 209453_at | M81768 | 0.125 | SLC9A1 |
| 364 | 214 | 208193_at | NM_000590 | 0.145 | IL9 |
| 365 | 223 | 208042_at | NM_013303 | 0.135 | HSU84971 |
| 366 | 364 | 206826_at | NM_002677 | 0.16 | PMP2 |
| 367 | 119 | 206592_s_at | NM_003938 | 0.16 | AP3D1 |
| 368 | 95 | 209480_at | M16276 | 0.175 | HLA-DQB1 |
| 369 | 228 | 212882_at | AB018338 | 0.17 | KIAA0795 |
| 370 | 339 | 215634_at | AF007137 | 0.185 | — |
| 371 | 97 | 214613_at | AW024085 | 0.185 | GPR3 |
| 372 | 9 | 206376_at | NM_018057 | 0.165 | NTT73 |
| 373 | 102 | 217109_at | AJ242547 | 0.175 | MUC4 |
| 374 | 276 | 209188_x_at | BC002809 | 0.2 | DR1 |
| 375 | 417 | 210567_s_at | BC001441 | 0.175 | SKP2 |
| 376 | 346 | 217056_at | X61070 | 0.195 | — |
| 377 | 258 | 207675_x_at | NM_003976 | 0.155 | ARTN |
| 378 | 328 | 214642_x_at | AI200443 | 0.165 | MAGEA5 |
| 379 | 183 | 216867_s_at | X03795 | 0.195 | PDGFA |
| 380 | 208 | 204849_at | NM_006602 | 0.195 | TCFL5 |
| 381 | 135 | 216939_s_at | Y08756 | 0.205 | HTR4 |
| 382 | 23 | 209530_at | U07139 | 0.2 | CACNB3 |
| 383 | 15 | 215175_at | AB023212 | 0.21 | PCNX |
| 384 | 185 | 209651_at | BC001830 | 0.195 | TGFB1I1 |
| 385 | 292 | 205339_at | NM_003035 | 0.225 | SIL |
| 386 | 287 | 209737_at | AB014605 | 0.205 | AIP1 |
| 387 | 186 | 206616_s_at | AF155382 | 0.225 | ADAM22 |
| 388 | 201 | 212819_at | AF055024 | 0.22 | ASB1 |
| 389 | 35 | 205498_at | NM_000163 | 0.205 | GHR |
| 390 | 239 | 214111_at | AF070577 | 0.195 | OPCML |
| 391 | 21 | 213106_at | AI769688 | 0.21 | ATP8A1 |
| 392 | 368 | 214456_x_at | M23699 | 0.225 | SAA2 |
| 393 | 221 | 200797_s_at | AI275690 | 0.235 | MCL1 |
| 394 | 115 | 213815_x_at | AI913329 | 0.24 | NY-REN-24 |
| 395 | 3 | 215935_at | AL080148 | 0.235 | DKFZP434B204 |
| 396 | 52 | 204316_at | W19676 | 0.235 | RGS10 |
| 397 | 17 | 216875_x_at | X83412 | 0.215 | HAB1 |
| 398 | 409 | 215996_at | AI446234 | 0.215 | — |
| 399 | 48 | 202555_s_at | NM_005965 | 0.2 | MYLK |
| 400 | 190 | 216993_s_at | U32169 | 0.255 | COL11A2 |
| 401 | 385 | 212461_at | BF793951 | 0.235 | OAZIN |
| 402 | 63 | 215781_s_at | D87012 | 0.25 | TOP3B |
| 403 | 99 | 209361_s_at | BC004153 | 0.215 | PCBP4 |
| 404 | 330 | 213363_at | AW170549 | 0.235 | na |
| 405 | 78 | 214948_s_at | AL050136 | 0.245 | — |
| 406 | 159 | 207222_at | NM_003561 | 0.27 | PLA2G10 |
| 407 | 100 | 213840_s_at | R68573 | 0.23 | MRPS12 |
| 408 | 145 | 210306_at | U89358 | 0.285 | L3MBTL |
| 409 | 123 | 206328_at | NM_004933 | 0.23 | CDH15 |
| 410 | 245 | 211108_s_at | U31601 | 0.28 | JAK3 |
| 411 | 134 | 213221_s_at | AB018324 | 0.27 | KIAA0781 |
| 412 | 198 | 212561_at | AA349595 | 0.27 | RAB6IP1 |
| 413 | 189 | 208237_x_at | AF155381 | 0.28 | ADAM22 |
| 414 | 139 | 217060_at | U03115 | 0.235 | — |
| 415 | 176 | 220937_s_at | NM_014403 | 0.265 | SIAT7D |
| 416 | 169 | 210345_s_at | AF257737 | 0.29 | DNAH9 |
| 417 | 323 | 206577_at | NM_003381 | 0.33 | VIP |
| 418 | 4 | 204919_at | NM_007244 | 0.29 | PROL4 |
| 419 | 55 | 216283_s_at | X64116 | 0.285 | PVR |
| 420 | 77 | 214096_s_at | AW190316 | 0.27 | LOC56901 |
| 421 | 32 | 216224_s_at | AK024083 | 0.31 | HDAC6 |
| 422 | 274 | 211349_at | AB001328 | 0.34 | SLC15A1 |
| 423 | 50 | 215766_at | AL096729 | 0.335 | GSTA1 |
| 424 | 281 | 216252_x_at | Z70519 | 0.32 | TNFRSF6 |
| 425 | 163 | 214463_x_at | NM_003541 | 0.365 | HIST1H4K |
| 426 | 219 | 209245_s_at | AB014606 | 0.295 | KIF1C |
| 427 | 52 | 204316_at | W19676 | 0.31 | RGS10 |
| 428 | 237 | 207044_at | NM_000461 | 0.31 | THRB |
| 429 | 261 | 207143_at | NM_001259 | 0.39 | CDK6 |
| 430 | 216 | 214872_at | AL080129 | 0.3 | DKFZP434D193 |
| 431 | 103 | 217110_s_at | AJ242547 | 0.475 | MUC4 |

Table 4: Shown are the average errors of the differentiating genes in predicting a prognosis (poor or good clinical outcome) of the MS test group based on a model computed for each gene or a group of genes in the MS training set group. The ascending order of genes reflects combinations of genes, where each row includes the gene specified in that row and in all preceding rows. For example, the average error presented in row number 4 reflects the average error in predicting clinical outcome of MS of the group of genes described in 1, 2, 3 and 4 (i.e., SEQ ID NOs: 158, 68, 5 and 58). Probeset ID = Affymetrix ID.

As shown in Table 4 hereinabove, the predictive power of each set of genes was evaluated using the MS training and test sets of samples. The polynucleotide exhibiting the best predictive power in determining MS prognosis (i.e., poor or good prognosis/clinical outcome) was the polynucleotide set forth by SEQ ID NO:158 (GenBank Accession No. NM_{—}005012; row No. 1 in Table 4), in which the average error between the test and training groups was “0” (zero) (100% accuracy). Similarly, the combination genes set forth by SEQ ID NOs: 158 and 68 (GenBank Accession No. NM_{—}001023; row No. 2 in Table 4) displayed a predictive power with “0” average error. Another exemplary combination is shown in row number 4 in Table 4, in which the combination of the polynucleotides set forth by SEQ ID NOs:158, 68, 5 and 58 displayed a high predictive power with “0” average error. Thus, this analysis enables one skilled in the art to select a group of polynucleotides which can give the best predictive power for the clinical outcome/prognosis of MS subjects.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References are Cited in Text

1. Confavreux C, Vukusic S. Natural history of multiple sclerosis: implications for counselling and therapy. Curr Opin Neurol 2002; 15: 257-66.
2. Trojano M, Paolicelli D, Bellacosa A, Cataldo S. The transition from relapsing-remitting MS to irreversible disability: clinical evaluation. Neurol Sci 2003; 24 (Suppl 5): S268-70.
3. Simon J H. Contrast-enhanced MR imaging in the evaluation of treatment response and prediction of outcome in multiple sclerosis. J Magn Reson Imaging 1997; 7:29-37.
4. Benedict R H, Weinstock-Guttman B, Fishman I, Sharma J, Tjoa C W, Bakshi R. Prediction of neuropsychological impairment in multiple sclerosis: comparison of conventional magnetic resonance imaging measures of atrophy and lesion burden. Arch Neurol 2004; 61:226-30.
5. Mantripragada K K, Buckley P G, de Stahl T D, Dumanski J P. Genomic microarrays in the spotlight. Trends Genet 2004; 20: 87-94.
6. Achiron A, Gurevich M, Friedman N, Kaminski N, Mandel M. Blood transcriptional signatures of multiple sclerosis: unique gene expression of disease activity. Ann Neurol 2004; 55: 410-7.
7. Kurtzke J F. Rating neurologic impairment in multiple sclerosis: an expanded disability status scale (EDSS). Neurology 1983; 33: 1444-52.
8. Li C, Wong W H. Model-based analysis of oligonucleotide arrays: expression index computation and outlier detection. Proc Natl Acad Sci USA 2001; 98: 31-6.
9. Eisen M B, Spellman P T, Brown P O, Botstein D. Cluster analysis and display of genome-wide expression patterns. Proc Natl Acad Sci USA 1998; 95: 14863-8.
10. Kaminski N, Friedman N. Practical approaches to analyzing results of microarray experiments. Am J Respir Cell Mol Biol 2002; 27: 125-32.
11. Ben-Dor A, Bruhn L, Friedman N, Nachman I, Schummer M, Yakhini Z. Tissue classification with gene expression profiles. J Comput Biol 2000; 7: 559-83.
12. Furey T S, Cristianini N, Duffy N, Bednarski D W, Schummer M, Haussler D. Support vector machine classification and validation of cancer tissue samples using microarray expression data. Bioinformatics 2000; 16:906-14.
13. Statnikov A, Aliferis C F, Tsamardinos I, Hardin D, Levy S. A comprehensive evaluation of multicategory classification methods for microarray gene expression cancer diagnosis. Bioinformatics 2005; 21:631-43.
14. Jain A K, Zongker D. Feature selection-evaluation, application, and small sample performance. IEEE Trans. On Pattern Analysis and Machine Intelligence 1997; 19:153-158.
15. Aha D W, Bankert R L. A comparative evaluation of sequential feature selection algorithms. In: Proceedings of 5th International Workship on Artificial Intelligence and statistics. D. Fisher, J. H. Lenx (Eds.), New York: Springer-Verlag, 1995, pp 1-7.
16. Goodkin D E, Hertsgaard D, Rudick R A. Exacerbation rates and adherence to disease type in a prospectively followed-up population with multiple sclerosis. Implications for clinical trials. Arch Neurol 1989; 46: 1107-12.
17. Kurtzke J F, Beebe G W, Nagler B, Kurland L T, Auth T L. Studies on the natural history of multiple sclerosis—8. Early prognostic features of the later course of the illness. J Chronic Dis 1977; 30: 819-30.
18. Weinshenker B G. The natural history of multiple sclerosis: update 1998. Semin Neurol 1998; 18: 301-7.
19. Weinshenker B G, Rice G P A, Noseworthy J H, Carriere W, Baskerville J, Ebers G C. The natural history of multiple sclerosis: a geographically bases study: 3. Multivariate analysis of predictive factors and models of outcome. Brain 1991; 114:1045-56.
20. Runmarker B, Andersen O. Prognostic factors in a multiple sclerosis incidence cohort with 25 years of follow-up. Brain 1993; 116:117-34.
21. Kantarci O H, Weinshenker B G. Prognostic factors in multiple sclerosis. In: Cook D S. (Ed.), Handbook of Multiple Sclerosis, 3rded, Marcel and Dekker, New York, 2001, pp. 449-63.
22. Tremlett H, Paty D, Devonshire V. Disability progression in multiple sclerosis is slower than previously reported. Neurology 2006; 66:172-7.
23. Zhang W, Geiman D E, Shields J M, Dang D T, Mahatan C S, Kaestner K H, Biggs J R, Kraft A S, Yang V W. The gut-enriched Kruppel-like factor (Kruppel-like factor 4) mediates the transactivating effect of p53 on the p21WAF1/Cipl promoter. J Biol Chem 2000; 275:18391-8.
24. Chen Z Y, Shie J L, Tseng C C. STAT1 is required for IFN-gamma-mediated gut-enriched Kruppel-like factor expression. Exp Cell Res. 2002; 281:19-27.
25. Petzold A, Eikelenboom M J, Gveric D, Keir G, Chapman M, Lazeron R H, Cuzner M L, Polman C H, Uitdehaag B M, Thompson E J, Giovannoni G. Markers for different glial cell responses in multiple sclerosis: clinical and pathological correlations. Brain 2002; 125(Pt 7):1462-73.
26. Petzold A, Brassat D, Mas P, Rejdak K, Keir G, Giovannoni G, Thompson E J, Clanet M. Treatment response in relation to inflammatory and axonal surrogate marker in multiple sclerosis. Mult Scler 2004; 10:281-3.
27. Faffe D S, Whitehead T, Moore P E, Baraldo S, Flynt L, Bourgeois K, Panettieri R A, Shore S A. IL-13 and IL-4 promote TARC release in human airway smooth muscle cells: role of IL-4 receptor genotype. Am J Physiol Lung Cell Mol Physiol 2003; 285:L907-14.
28. Dabbagh K, Takeyama K, Lee H M, Ueki I F, Lausier J A, Nadel J A. IL-4 induces mucin gene expression and goblet cell metaplasia in vitro and in vivo. J Immunol 1999; 162:6233-7.
29. Zhu Z, Lee C G, Zheng T, Chupp G, Wang J, Homer R J, Noble P W, Hamid Q, Elias J A. Airway inflammation and remodeling in asthma. Lessons from interleukin 11 and interleukin 13 transgenic mice. Am J Respir Crit Care Med 2001; 164 (10 Pt 2):567-70.
30. Soto P, Price-Schiavi S A, Carraway K L. SMAD2 and SMAD7 involvement in the post-translational regulation of Muc4 via the transforming growth factor-beta and interferon-gamma pathways in rat mammary epithelial cells. J Biol Chem 2003; 278:20338-44.
31. Achour A, Laaroubi D, Caruelle D, Barritault D, Courty J. The angiogenic factor heparin affin regulatory peptide (HARP) induces proliferation of human peripheral blood mononuclear cells. Cell Mol Biol 2001; 47:OL73-7.
32. Heroult M, Bernard-Pierrot I, Delbe J, Hamma-Kourbali Y, Katsoris P, Barritault D, Papadimitriou E, Plouet J, Courty J. Heparin affin regulatory peptide binds to vascular endothelial growth factor (VEGF) and inhibits VEGF-induced angiogenesis. Oncogene 2004; 23:1745-53.

What is claimed is:

1. A composition of matter comprising a polynucleotide sample of a subject diagnosed with multiple sclerosis, and an oligonucleotide which specifically hybridizes with at least one polynucleotide sequence selected from the group consisting of SEQ ID NOs: 1-431.

2. The composition of matter of claim 1, wherein said oligonucleotide is labeled.

3. The composition of matter of claim 1, wherein said polynucleotide sample is obtained from a blood sample of said subject.

4. The composition of matter of claim 1, wherein said at least one polynucleotide sequence is selected from the group consisting of SEQ ID NOs: 1-193.

5. The composition of matter of claim 1, wherein said at least one polynucleotide sequence is selected from the group consisting of SEQ ID NOs: 194-431.

6. The composition of matter of claim 1, wherein said at least one polynucleotide sequence is selected from the group consisting of SEQ ID NOs: 156, 143, 127, 46, 311, 140, 74, 276, 180, 182, 191, 61, 306, 115, 97, 303, 272, 50, 16, 63, 117, 406, 423, 128, 277, 47, 17, 424, 418, 190, 139, 102, 103 and 325.

7. The composition of matter of claim 1, wherein said at least one polynucleotide sequence is selected from the group consisting of SEQ ID NOs: 127, 423, 16, 17, 424, 190 and 325.

8. The composition of matter of claim 1, wherein said at least one polynucleotide sequence is set forth in SEQ ID NO: 424.

9. The composition of matter of claim 1, wherein said at least one polynucleotide sequence is selected from the group consisting of SEQ ID NOs: 158, 68, 5, 58, 329 and 120.

10. The composition of matter of claim 1, wherein said at least one polynucleotide sequence is set forth in SEQ ID NO: 158.

11. A composition of matter comprising a polypeptide sample of a subject diagnosed with multiple sclerosis, and an antibody capable of specifically binding at least one polypeptide encoded by at least one polynucleotide sequence selected from the group consisting of SEQ ID NOs: 1-431.

12. The composition of matter of claim 11, comprising a secondary antibody.

13. The composition of matter of claim 11, wherein said polypeptide sample is obtained from a blood sample of said subject.

14. The composition of matter of claim 11, wherein said at least one polynucleotide sequence is selected from the group consisting of SEQ ID NOs: 1-193.

15. The composition of matter of claim 11, wherein said at least one polynucleotide sequence is selected from the group consisting of SEQ ID NOs: 194-431.

16. The composition of matter of claim 11, wherein said at least one polynucleotide sequence is selected from the group consisting of SEQ ID NOs: 156, 143, 127, 46, 311, 140, 74, 276, 180, 182, 191, 61, 306, 115, 97, 303, 272, 50, 16, 63, 117, 406, 423, 128, 277, 47, 17, 424, 418, 190, 139, 102, 103 and 325.

17. The composition of matter of claim 11, wherein said at least one polynucleotide sequence is selected from the group consisting of SEQ ID NOs: 127, 423, 16, 17, 424, 190 and 325.

18. The composition of matter of claim 11, wherein said at least one polynucleotide sequence is set forth in SEQ ID NO: 424.

19. The composition of matter of claim 11, wherein said at least one polynucleotide sequence is selected from the group consisting of SEQ ID NOs: 158, 68, 5, 58, 329 and 120.

20. The composition of matter of claim 11, wherein said at least one polynucleotide sequence is set forth in SEQ ID NO: 158.
